(19) United States
(12) Patent Application Publication
(10) Pub. No.: US 2025/0051279 A1
(43) Pub. Date: Feb. 13, 2025

(54) TRYPTAMINE COMPOSITIONS AND METHODS

(71) Applicant: Cybin IRL Limited, Dublin (IE)

(72) Inventors: Kenneth L. Avery, Merrimack, NH (US); James He Huang, Parkland, FL (US); Alex Nivorozhkin, West Roxbury, MA (US); Pradip M. Pathare, Lexington, MA (US); Mohammed I. Shukoor, Lexington, MA (US)

(73) Assignee: Cybin IRL Limited, Dublin (IE)

(21) Appl. No.: 18/720,922

(22) PCT Filed: Jan. 13, 2023
(86) PCT No.: PCT/EP2023/050702

§ 371 Date: Jun. 17, 2024

Related U.S. Application Data
(60) Provisional application No. 63/384,704, filed on Nov. 22, 2022.
(60) Provisional application No. 63/299,599, filed on Jan. 14, 2022.

(51) Int. Cl. C07D 209/16; A61K 9/00; A61K 31/4045; A61K 47/02; C07B 59/00

(52) U.S. Cl. CPC C07D 209/16; A61K 9/0078; A61K 31/4045; A61K 47/02; C07B 59/002

(57) ABSTRACT

There are disclosed pharmaceutically acceptable salts of tryptamine compounds, the use of such salt forms in the treatment of diseases associated with a serotonin 5-HT₂ receptor, pharmaceutical compositions such as those adapted for inhalation administration containing the salt forms, methods of delivering the pharmaceutically acceptable salt forms (e.g., via inhalation), and methods of treating diseases or disorders associated with a serotonin 5-HT₂ receptor, such as central nervous system (CNS) disorders or psychological disorders, with the salt forms. (Formula (I))

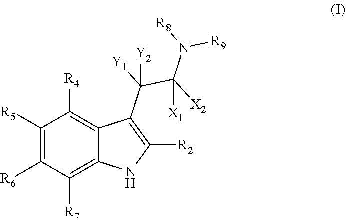

### CROSS-REFERENCE

This application claims priority to U.S. Provisional Application No. 63/299,599, filed on Jan. 14, 2022, and U.S. Provisional Application No. 63/384,704, filed on Nov. 22, 2022, each incorporated by reference herein in their entireties.

### FIELD

The present disclosure relates generally to pharmaceutically acceptable salts of tryptamine compounds and, in some embodiments, to serotonin 5-HT₂ receptor agonists and uses in the treatment of diseases associated with a 5-HT₂ receptor.

### BACKGROUND

There are three, closely related subtypes of serotonin 5-HT₂ receptors (5-HT₂Rs), 5-HT_{2A}, 5-HT_{2B}, and 5-HT_{2C}, and they are primary targets of classic serotonergic psychedelics, such as lysergic acid diethylamide (LSD), psilocybin, and 2,5-dimethoxy-4-bromoamphetamine (DOB). They share approximately 60% transmembrane amino acid homology, which poses a challenge to design molecules with selectivity for one subtype over the others. Each subtype is expressed in a unique pattern in mammals (both in peripheral tissues and in the central nervous system), and when stimulated, produces unique biochemical, physiological, and behavioral effects. Activation of 5-HT_{2A}Rs, for example, predominantly mediates psychedelic effects and elicits anti-inflammatory effects, whereas activation of 5-HT_{2C}Rs reduces feeding behavior. Chronic activation of 5-HT_{2B}Rs, however, has been linked to valvular heart disease (VHD), a life-threatening adverse event (AE). Furthermore, there are concerns that patients who could benefit from a 5-HT_{2A}R pharmacotherapy could be resistant to experiencing psychedelic effects.

Tryptamines are a class of serotonergic psychedelics, and possess very high potencies at serotonin 5-HT₂Rs (in some cases sub-nanomolar affinities). Certain tryptamines are distinguished from classic psychedelics and other serotonergic psychedelics by possessing selectivity—in some cases 100-fold—for 5-HT_{2A}Rs over 5-HT_{2B}Rs and 5-HT_{2C}Rs.

AEs caused by tryptamines and other serotonergic psychedelics are associated with ingestion of relatively high doses. Likely owing to their very high potency at 5-HT_{2A}Rs and 5-HT_{2C}Rs, the active oral doses of tryptamines and other psychedelics are extremely low. For example, 2-(4-Chloro-2,5-dimethoxyphenyl)-N-[(2-methoxyphenyl)methyl]ethan-1-amine (2C—C-NBOMe) is orally active at doses as low 25 μg, and very strong psychedelic doses are in the range of 500-700 μg. Thus, misuse or abuse, at or exceeding these doses, can cause a negative experience for the patient, presenting as acute psychedelic crisis, colloquially known as a “bad trip,” in which the patient experiences feelings of visual and auditory hallucinations, agitation, aggressiveness, psychosis, remorse, and distress. Poisoning has also been associated with toxicity (e.g., rhabdomyolysis) and fatalities. Tryptamines such as N,N-dimethyltryptamine (DMT) (IUPAC: 2-(1H-indol-3-yl)-N,N-dimethylethan-1-amine)) can also undergo extensive first-pass metabolism, rendering them orally inactive.

Therefore, the therapeutic index of many tryptamine psychedelics is relatively narrow. So maximizing therapeutic benefits of potential drug candidate molecules requires fine-tuning of the dose and route of administration, along with dose titration, to reduce the side effects and improve safety.

Further, like many free base form pharmaceuticals, tryptamines in free base form are generally not well suited for pharmaceutical processing and applications. For example, DMT free base is a low melting solid (approximately 46° C.) and is known to be sensitive to oxygen, heat, and light under prolonged storage.

Accordingly, there is a need for tryptamines that overcome the 5-HT_{2B}R problem and the issue of adverse events, as well as a need to improve their bioavailability and enhance their activity and exposure. There is a further need for efficient, more convenient, and controllable tryptamine formulations that afford no neurologically toxic (e.g., psychotomimetic toxic) plasma concentration. There is a further need for stabilized and physiological acceptable forms of tryptamines suitable for pharmaceutical preparation and administration.

### SUMMARY

The present disclosure is based at least in part on the identification of salt forms of compounds that modulate serotonin 5-HT₂ receptors and methods of using the same to treat diseases associated with a serotonin 5-HT₂ receptor. More specifically, the present disclosure provides novel salt forms of N,N-dimethyltryptamine (DMT) and derivatives thereof that possess advantageous physical and pharmaceutical characteristics, and that permit e.g., efficient, more convenient, and controllable pharmaceutical formulations to be prepared for the treatment of diseases/conditions such as a neuropsychiatric disease or disorder, an inflammatory disease or disorder, a central nervous system (CNS) disorder, an autonomic nervous system (ANS) disorder, a pulmonary disorder, and/or a cardiovascular disorder.

These and other objects, which will become apparent during the following detailed description, have been achieved by the inventors' discovery of novel salt forms of compounds of Formula (I) which possess desirable physical and pharmaceutical characteristics such as one or more of: ease and propensity for salt formation and crystallization (e.g., high yield); stable and well-defined physical properties (e.g., crystallinity, lack of polymorphism, high melting point, and high enthalpy of fusion); stability against moisture including at elevated humidity (hygroscopicity); desirable appearance (e.g., free flowing, lack of cohesion/adhesion, regular morphology); acceptable aqueous solubility; and physiological acceptability, particularly for pulmonary administration (e.g., non-irritant).

Such suitable salt forms of the compounds described herein (e.g., compounds of Formula (I)) can thus be administered, e.g., via inhalation in a controlled manner, with fast absorption and rapid onset of action provided by the massive surface area of the alveolar region, the abundant vasculature, and thin air-blood barrier, while avoiding first pass metabolism, thereby enabling the use of relatively low doses, low incidence of systemic side effects, and more advantageous pharmacokinetic profiles. In particular, pulmonary administration of suitable salt forms of the compounds described herein (e.g., compounds of Formula (I)) in combination with a N-methyl-D-aspartate (NMDA) receptor antagonist (e.g., nitrous oxide-a dissociative anesthetic which provides a calming and euphoric feeling, and which controls and/or reduces the activating effects of the 5-HT₂Rs), can reduce the risk of overstimulation and thus the occurrences of psychiatric adverse effects such as acute psychedelic crisis.

Thus, the present disclosure provides:

(1) A pharmaceutically acceptable salt of a compound of Formula (I), or a solvate thereof,

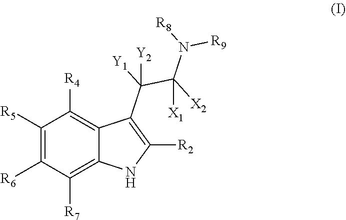

wherein:
X₁ and X₂ are deuterium,
Y₁ and Y₂ are deuterium,
R₂, R₄, R₅, R₆, and R₇ are independently hydrogen or deuterium, and R₈ and R₉ are independently selected from the group consisting of —CH₃, —CDH₂, —CD₂H, and —CD₃.

(2) The pharmaceutically acceptable salt of (1), wherein at least one of R₈ and R₉ comprises deuterium.

(3) The pharmaceutically acceptable salt of (1) or (2), wherein R₂, R₄, R₅, R₆, and R₇ are hydrogen.

(4) The pharmaceutically acceptable salt of (1) or (2), wherein at least one of R₂, R₄, R₅, R₆, and R₇ are deuterium.

(5) The pharmaceutically acceptable salt of any one of (1) to (4), wherein R₈ and R₉ are —CH₃.

(6) The pharmaceutically acceptable salt of any one of (1) to (4), wherein R₈ and R₉ are independently selected from the group consisting of —CDH₂, —CD₂H, and —CD₃.

(7) The pharmaceutically acceptable salt of any one of (1) to (4), wherein R₈ and R₉ are —CD₃.

(8) The pharmaceutically acceptable salt of any one of (1) to (7), wherein the compound of Formula (I) is selected from the group consisting of

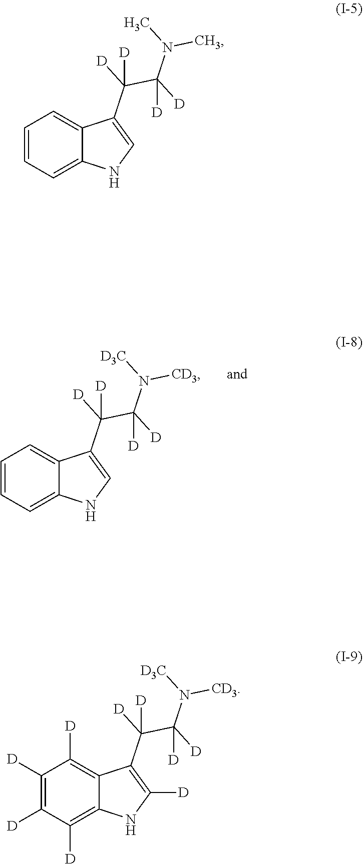 [SMILES: [2H]C([2H])(c1c[nH]c2ccccc12)C([2H])([2H])N(C)C ; [2H]C([2H])([2H])N(C([2H])([2H])[2H])C([2H])([2H])C([2H])([2H])c1c[nH]c2ccccc12 ; [2H]c1[nH]c2c([2H])c([2H])c([2H])c([2H])c2c1C([2H])([2H])C([2H])([2H])N(C([2H])([2H])[2H])C([2H])([2H])[2H]]

(9) The pharmaceutically acceptable salt of (1), wherein the compound of Formula (I) is

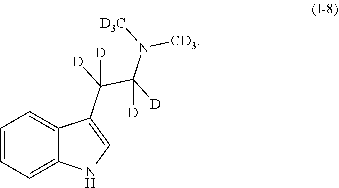 [SMILES: [2H]C([2H])([2H])N(C([2H])([2H])[2H])C([2H])([2H])C([2H])([2H])c1c[nH]c2ccccc12]

(10) The pharmaceutically acceptable salt of any one of (1) to (9), which is crystalline, as determined by X-ray powder diffraction (XRPD).

(11) The pharmaceutically acceptable salt of any one of (1) to (10), which has a water solubility from about 10 mg/mL to about 400 mg/mL.

(12) The pharmaceutically acceptable salt of any one of (1) to (11), which has a melt onset from about 100° C. to about 210° C., as determined by differential scanning calorimetry (DSC).

(13) The pharmaceutically acceptable salt of any one of (1) to (12), which has an enthalpy of fusion from about 110 J·g⁻¹ to about 180 J·g⁻¹ as determined by differential scanning calorimetry (DSC).

(14) The pharmaceutically acceptable salt of any one of (1) to (13), which has a weight increase of less than 1% w/w when exposed to a relative humidity (RH) of >95% RH, as determined by dynamic vapor sorption (DVS).

(15) The pharmaceutically acceptable salt of any one of (1) to (14), which is an addition salt of the compound of Formula (I) with an organic acid.

(16) The pharmaceutically acceptable salt of any one of (1) to (15), which is a fumarate, a benzoate, a salicylate, or a succinate salt of the compound of Formula (I).

(17) The pharmaceutically acceptable salt of any one of (1) to (16), which is a fumarate salt of the compound of Formula (I).

(18) The pharmaceutically acceptable salt of any one of (1) to (16), which is a benzoate salt of the compound of Formula (I).

(19) The pharmaceutically acceptable salt of any one of (1) to (16), which is a salicylate salt of the compound of Formula (I).

(20) The pharmaceutically acceptable salt of any one of (1) to (16), which is a succinate salt of the compound of Formula (I).

(21) The pharmaceutically acceptable salt of any one of (1) to (16), which is a fumarate salt of 2-(1H-indol-3-yl)-N,N-bis(methyl-d₃)ethan-1-amine-1,1,2,2-d₄ (I-8a).

(22) The pharmaceutically acceptable salt of (21), which is in a crystalline solid form characterized by an X-ray powder diffraction pattern containing at least three characteristic peaks at diffraction angles (2θ±0.2°) selected from 7.8°, 10.3°, 10.9°, 13.6°, 15.8°, 16.10, 17.0°, 18.4°, 19.7°, 19.9°, 20.6°, 21.3°, 21.8°, 22.5°, 23.8°, 24.1°, 25.1°, 26.2°, 33.6° and 34.9° as determined by XRPD using a CuKα radiation source.

(23) The pharmaceutically acceptable salt of any one of (1) to (16), which is a benzoate salt of 2-(1H-indol-3-yl)-N,N-bis(methyl-d₃)ethan-1-amine-1,1,2,2-d₄ (I-8b).

(24) The pharmaceutically acceptable salt of (23), which is in a crystalline solid form characterized by an X-ray powder diffraction pattern containing at least three characteristic peaks at diffraction angles (2θ±0.2°) selected from 9.6°, 11.10, 12.7°, 13.5°, 15.8°, 16.10, 17.2°, 17.9°, 19.8°, 20.1°, 20.8°, 21.2°, 22.8°, 23.8°, 24.6°, 26.9°, 29.3°, 32.3°, 35.1°, and 36.1°, as determined by XRPD using a CuKα radiation source.

(25) The pharmaceutically acceptable salt of any one of (1) to (16), which is a salicylate salt of 2-(1H-indol-3-yl)-N,N-bis(methyl-d₃)ethan-1-amine-1,1,2,2-d₄ (I-8c).

(26) The pharmaceutically acceptable salt of (25), which is in a crystalline solid form characterized by an X-ray powder diffraction pattern containing at least three characteristic peaks at diffraction angles (2θ±0.2°) selected from 9.6°, 10.5°, 14.9°, 17.10, 18.10, 19.10, 20.10, 20.8°, 21.1°, 21.3°, 24.6°, 25.6°, 28.5°, 28.8°, 29.4°, 30.3°, 31.3°, 32.1°, 33.5° and 34.4° as determined by XRPD using a CuKα radiation source.

(27) The pharmaceutically acceptable salt of any one of (1) to (16), which is a succinate salt of 2-(1H-indol-3-yl)-N,N-bis(methyl-d₃)ethan-1-amine-1,1,2,2-d₄ (I-8d).

(28) A pharmaceutical composition, comprising the pharmaceutically acceptable salt of any one of (1) to (27) and a pharmaceutically acceptable vehicle.

(29) The pharmaceutical composition of (28), wherein any position in the compound of Formula (I) having deuterium has a minimum deuterium incorporation of at least 50 atom % at the site of deuteration.

(30) The pharmaceutical composition of (28) or (29), which is adapted for administration via inhalation.

(31) The pharmaceutical composition of (28) or (29), which is adapted for oral administration.

(32) The pharmaceutical composition of (28) or (29), which is adapted for intravenous administration.

(33) The pharmaceutical composition of (28) or (29), which is adapted for subcutaneous administration.

(34) The pharmaceutical composition of (28) or (29), which is adapted for intramuscular administration.

(35) The pharmaceutical composition of (28) or (29), which is adapted for nasal administration.

(36) A liquid dosage form, prepared by reconstituting the pharmaceutical composition of (28) or (29) in solid dosage form, in a pharmaceutically acceptable liquid medium.

(37) A method of delivering the pharmaceutically acceptable salt of any one of (1) to (27) to a patient in need thereof, comprising:
administering an aerosol to the patient by inhalation, wherein the aerosol comprises the pharmaceutically acceptable salt of the compound of Formula (I) in a carrier.

(38) The method of (37), wherein the pharmaceutically acceptable salt is delivered to the patient's central nervous system via pulmonary absorption.

(39) The method of (37) or (38), wherein the carrier is air, oxygen, or a mixture of helium and oxygen.

(40) The method of (39), wherein the carrier is the mixture of helium and oxygen.

(41) The method of (40), wherein the mixture of helium and oxygen is heated to about 50° C. to about 60° C.

(42) The method of (40) or (41), wherein the helium is present in the mixture of helium and oxygen at about 50% to 90% by volume, and the oxygen is present in the mixture of helium and oxygen at about 50% to 10% by volume.

(43) The method of any one of (37) to (42), further comprising administering a pretreatment inhalation therapy prior to administration of the aerosol comprising the pharmaceutically acceptable salt of the compound of Formula (I) and the carrier.

(44) The method of (43), wherein the pretreatment inhalation therapy comprises administering via inhalation a mixture of helium and oxygen heated to about 90° C. to about 120° C. to the patient.

(45) The method of (44), comprising (i) administering via inhalation the mixture of helium and oxygen heated to about 90° C. to about 120° C. to the patient, and (ii) administering via inhalation to the patient an aerosol comprising the pharmaceutically acceptable salt of the compound of Formula (I) in a mixture of helium and oxygen heated to about 50° C. to about 60° C.

(46) The method of (45), further comprising repeating steps (i) and (ii) 1 to 5 times.

(47) The method of any one of (37) to (46), wherein the pharmaceutically acceptable salt of the compound of Formula (I) is delivered to the patient's central nervous system, providing an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

(48) The method of any one of (37) to (47), wherein the aerosol is a mist.

(49) The method of any one of (37) to (48), wherein the aerosol is prepared by nebulization of the pharmaceutically acceptable salt of the compound of Formula (I).

(50) The method of (49), wherein the nebulization is performed with a device selected from the group consisting of a jet nebulizer, an ultrasonic nebulizer, a breath-actuated nebulizer, and a vibrating mesh nebulizer.

(51) The method of (49) or (50), wherein the nebulization is performed using a driving gas comprising nitrous oxide for entrainment of the nebulized form of the pharmaceutically acceptable salt of the compound of Formula (I).

(52) The method of (51), wherein the nitrous oxide is present in the driving gas at a concentration of 15 to 25 vol %, relative to a total volume of the driving gas.

(53) The method of any one of (37) to (52), wherein the aerosol is administered for 20 to 60 minutes.

(54) A method of treating a patient with a central nervous system (CNS) disorder and/or psychological disorder, comprising:
administering to the patient a therapeutically effective amount of the pharmaceutically acceptable salt of any one of (1) to (27).

(55) The method of (54), wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

(56) The method of (54), wherein the CNS disorder and/or psychological disorder is alcohol use disorder.

(57) The method of (54), wherein the CNS disorder and/or psychological disorder is generalized anxiety disorder (GAD).

(58) The method of (54), wherein the CNS disorder and/or psychological disorder is social anxiety disorder.

(59) The method of (54), wherein the CNS disorder and/or psychological disorder is treatment-resistant depression (TRD).

(60) A pharmaceutical composition, comprising:
an active salt mixture comprising (i) a pharmaceutically acceptable salt of 2-(1H-indol-3-yl)-N,N-bis(methyl-d₃)ethan-1-amine-1,1,2,2-d₄ (I-8), and (ii) a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and
a pharmaceutically acceptable vehicle.

(61) The pharmaceutical composition of (60), wherein the active salt mixture comprises (i) from 60% to 98% by weight of the pharmaceutically acceptable salt of 2-(1H-indol-3-yl)-N,N-bis(methyl-d₃)ethan-1-amine-1,1,2,2-d₄ (I-8), based on a total weight of the active salt mixture, and (ii) from 2% to 40% by weight, in sum, of the pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), based on a total weight of the active salt mixture.

(62) The pharmaceutical composition of (60) or (61), wherein the active salt mixture comprises (i) from 90% to 98% by weight of a pharmaceutically acceptable salt of 2-(1H-indol-3-yl)-N,N-bis(methyl-d₃)ethan-1-amine-1,1,2,2-d₄ (I-8), based on a total weight of the active salt mixture, and (ii) from 2% to 10% by weight, in sum, of the pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), based on a total weight of the active salt mixture.

(63) The pharmaceutical composition of any one of (60) to (62), wherein the active salt mixture comprises (i) a fumarate salt of 2-(1H-indol-3-yl)-N,N-bis(methyl-d₃)ethan-1-amine-1,1,2,2-d₄ (I-8a), and (ii) a fumarate salt of one or more of 2-(1H-indol-3-yl)-N,N-bis(methyl-d₃)ethan-1-amine-1,2,2-d₃ (I-10a) and/or 2-(1H-indol-3-yl)-N,N-bis(methyl-d₃)ethan-1-amine-1,1,2-d₃ (I-11a).

(64) The pharmaceutical composition of any one of (60) to (62), wherein the active salt mixture comprises (i) a benzoate salt of 2-(1H-indol-3-yl)-N,N-bis(methyl-d₃)ethan-1-amine-1,1,2,2-d₄ (I-8b), and (ii) a benzoate salt of one or more of 2-(1H-indol-3-yl)-N,N-bis(methyl-d₃)ethan-1-amine-1,2,2-d₃ (I-10b) and/or 2-(1H-indol-3-yl)-N,N-bis(methyl-d₃)ethan-1-amine-1,1,2-d₃ (I-11b).

(65) The pharmaceutical composition of any one of (60) to (62), wherein the active salt mixture comprises (i) a salicylate salt of 2-(1H-indol-3-yl)-N,N-bis(methyl-d₃)ethan-1-amine-1,1,2,2-d₄ (I-8c), and (ii) a salicylate salt of one or more of 2-(1H-indol-3-yl)-N,N-bis(methyl-d₃)ethan-1-amine-1,2,2-d₃ (I-10c) and/or 2-(1H-indol-3-yl)-N,N-bis(methyl-d₃)ethan-1-amine-1,1,2-d₃ (I-11c).

(66) The pharmaceutical composition of any one of (60) to (62), wherein the active salt mixture comprises (i) a succinate salt of 2-(1H-indol-3-yl)-N,N-bis(methyl-d₃)ethan-1-amine-1,1,2,2-d₄ (I-8d), and (ii) a succinate salt of one or more of 2-(1H-indol-3-yl)-N,N-bis(methyl-d₃)ethan-1-amine-1,2,2-d₃ (I-10d) and/or 2-(1H-indol-3-yl)-N,N-bis(methyl-d₃)ethan-1-amine-1,1,2-d₃ (I-11 d).

(67) A method of treating a patient with a central nervous system (CNS) disorder and/or psychological disorder, comprising:
administering to the patient, via inhalation, a therapeutically effective amount of an aerosol comprising the pharmaceutically acceptable salt of any one of (1) to (27) in a carrier.

(68) The method of (67), wherein the aerosol is a mist.

(69) The method of (67) or (68), wherein the carrier is air, oxygen, or a mixture of helium and oxygen.

(70) The method of (69), wherein the carrier is the mixture of helium and oxygen, and the mixture of helium and oxygen is heated to about 50° C. to about 60° C. prior to administering the aerosol to the patient.

(71) The method of any one of (67) to (70), wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

(72) The method of any one of (67) to (71), wherein the CNS disorder and/or psychological disorder is alcohol use disorder.

(73) The method of any one of (67) to (71), wherein the CNS disorder and/or psychological disorder is generalized anxiety disorder (GAD).

(74) The method of any one of (67) to (71), wherein the CNS disorder and/or psychological disorder is social anxiety disorder.

(75) The method of any one of (67) to (71), wherein the CNS disorder and/or psychological disorder is treatment-resistant depression (TRD).

(76) The method of any one of (67) to (75), wherein the aerosol is prepared by nebulization of the pharmaceutically acceptable salt of the compound of Formula (I).

(77) The method of (76), wherein the nebulization is performed with a device selected from the group consisting of a jet nebulizer, an ultrasonic nebulizer, a breath-actuated nebulizer, and a vibrating mesh nebulizer.

(78) The method of (76) or (77), wherein the nebulization is performed using a driving gas comprising nitrous oxide for entrainment of the nebulized form of the pharmaceutically acceptable salt of the compound of Formula (I).

(79) The method of (78), wherein the nitrous oxide is present in the driving gas at a concentration of 15 to 25 vol %, relative to a total volume of the driving gas.

(80) The method of any one of (67) to (79), wherein the aerosol is administered for 20 to 60 minutes.

(81) A method of delivering the pharmaceutically acceptable salt of any one of (1) to (27) to a patient in need thereof, comprising:
administering a dry powder to the patient by inhalation via a dry powder inhaler, wherein the dry powder comprises the pharmaceutically acceptable salt of the compound of Formula (I).

(82) The method of (81), wherein the dry powder comprises a particulate carrier having the pharmaceutically acceptable salt of the compound of Formula (I) on a surface thereof.

(83) The method of (82), wherein the pharmaceutically acceptable salt of the compound of Formula (I) is releasably absorbed onto the surface of the particulate carrier, such that upon inhalation by the patient, the compound of Formula (I) is released from the particulate carrier within the patient.

(84) The method of (81), wherein the dry powder is formed of the pharmaceutically acceptable salt of the compound of Formula (I) in solid particulate form.

(85) The method of any one of (81) to (84), wherein the pharmaceutically acceptable salt of the compound of Formula (I) is delivered to the patient's central nervous system via pulmonary absorption.

(86) The method of any one of (81) to (85), further comprising administering a pretreatment inhalation therapy prior to administration of the dry powder to the patient.

(87) The method of (86), wherein the pretreatment inhalation therapy comprises administering via inhalation a mixture of helium and oxygen heated to about 90° C. to about 120° C. to the patient.

(88) The method of (86) or (87), wherein the pretreatment inhalation therapy and the administering of the dry powder to the patient is repeated 1 to 5 times.

(89) The method of any one of (81) to (88), wherein the pharmaceutically acceptable salt of the compound of Formula (I) is delivered to the patient's central nervous system, providing an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

(90) A combination drug therapy, comprising:
the pharmaceutically acceptable salt of any one of (1) to (27); and
a N-methyl-D-aspartate (NMDA) receptor antagonist.

(91) The combination drug therapy of (90), wherein the NMDA receptor antagonist is at least one selected from the group consisting of ketamine, nitrous oxide, memantine, and dextromethorphan.

(92) The combination drug therapy of (90) or (91), wherein the NMDA receptor antagonist is nitrous oxide.

(93) The combination drug therapy of (92), wherein the pharmaceutically acceptable salt and the nitrous oxide are provided for administration as a single aerosol.

(94) The combination drug therapy of (92), wherein the pharmaceutically acceptable salt and the nitrous oxide are provided for administration as separate dosage forms.

(95) The combination drug therapy of (94), wherein the pharmaceutically acceptable salt is provided for administration as an aerosol, and the nitrous oxide is provided for administration as a therapeutic gas mixture.

(96) A method of treating a patient with a central nervous system (CNS) disorder and/or psychological disorder, comprising:
administering to the patient, via inhalation, a therapeutically effective amount of the pharmaceutically acceptable salt of any one of (1) to (27) and a N-methyl-D-aspartate (NMDA) receptor antagonist.

(97) The method of (96), wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

(98) The method of (96) or (97), wherein the CNS disorder and/or psychological disorder is alcohol use disorder.

(99) The method of (96) or (97), wherein the CNS disorder and/or psychological disorder is generalized anxiety disorder (GAD).

(100) The method of (96) or (97), wherein the CNS disorder and/or psychological disorder is social anxiety disorder.

(101) The method of (96) or (97), wherein the CNS disorder and/or psychological disorder is treatment-resistant depression (TRD).

(102) The method of any one of (96) to (101), wherein the NMDA receptor antagonist is at least one selected from the group consisting of ketamine, nitrous oxide, memantine, and dextromethorphan.

(103) The method of any one of (96) to (102), wherein the NMDA receptor antagonist is nitrous oxide.

(104) The method of (103), wherein the pharmaceutically acceptable salt and the nitrous oxide are administered to the patient as a single aerosol.

(105) The method of (103), wherein the pharmaceutically acceptable salt and the nitrous oxide are administered as separate dosage forms.

(106) The method of (105), wherein the pharmaceutically acceptable salt is administered as an aerosol, and the nitrous oxide is administered as a therapeutic gas mixture.

(107) The method of (105) or (106), wherein the pharmaceutically acceptable salt and the nitrous oxide are administered sequentially.

(108) The method of (105) or (106), wherein the pharmaceutically acceptable salt and the nitrous oxide are administered substantially simultaneously.

(109) An inhalation delivery device for delivery of a combination of nitrous oxide and the pharmaceutically acceptable salt of any one of (1) to (27) by inhalation to a patient in need thereof, comprising:
an inhalation outlet portal for administration of the combination of nitrous oxide and the pharmaceutically acceptable salt to the patient;
a container configured to deliver nitrous oxide gas to the inhalation outlet portal; and
a device configured to generate and deliver an aerosol comprising the pharmaceutically acceptable salt to the inhalation outlet portal.

(110) The inhalation delivery device of (109), wherein the inhalation outlet portal is a mouthpiece or a mask covering the patient's nose and mouth.

(111) The inhalation delivery device of (109) or (110), wherein the device configured to generate and deliver the aerosol to the inhalation outlet portal is a nebulizer.

(112) The inhalation delivery device of (111), wherein the nebulizer is a jet nebulizer and the nitrous oxide gas acts as a driving gas for the jet nebulizer.

(113) The inhalation delivery device of any one of (109) to (112), further comprising electronics configured to provide remote activation and operational control of the inhalation delivery device.

(114) Use of the pharmaceutically acceptable salt of any one of (1) to (27) for treating a patient with a central nervous system (CNS) disorder and/or psychological disorder.

(115) A pharmaceutically acceptable salt of any one of (1) to (27) for use in therapy.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIG. 1 illustrates a general synthetic route for making Compounds of Formula (I), e.g., compounds I-1, 1-2, 1-4, and I-6;

FIG. 2 illustrates a general synthetic route for making Compounds of Formula (I), e.g., compounds I-1, I-4, I-5, and I-8;

FIG. 3 shows X-ray powder diffractograms of Example 6 (I-1f; glycolate), Example 9 (I-1i; hemi-fumarate), and Example 1 (I-1a; fumarate);

FIG. 4 shows X-ray powder diffractograms of Example 2 (I-1b; benzoate), Example 3 (I-1c; salicylate), and Example 7 (I-1g; hemi-oxalate);

FIG. 5 shows X-ray powder diffractograms of Example 5 (I-1e; oxalate), Example 4 (I-1d; succinate), and Example 8 (I-1h; hemi-fumarate);

FIG. 6 shows an X-ray powder diffractogram of Example 26 (I-8a; fumarate) in comparison to Example 1 (I-1a; fumarate);

FIG. 7 shows an X-ray powder diffractogram of Example 27 (I-8b; benzoate) in comparison to Example 2 (I-1b; benzoate);

FIG. 8 shows an X-ray powder diffractogram of Example 28 (I-8c; salicylate) in comparison to Example 3 (I-1c; salicylate);

FIG. 9 shows the DSC curve of Example 1 (I-1a; fumarate);

FIG. 10 shows the DSC curve of Example 2 (I-1b; benzoate);

FIG. 11 shows the DSC curve of Example 4 (I-1d; succinate);

FIG. 12 shows the DSC curve of Example 5 (I-1e; oxalate);

FIG. 13 shows the DSC curve of Example 3 (I-1c; salicylate);

FIG. 14 shows the DSC curve of Example 6 (I-1f; glycolate);

FIG. 15 shows the DSC curve of Example 9 (I-1i; hemi-fumarate);

FIG. 16 shows the DSC curve of Example 8 (I-1h; hemi-fumarate);

FIG. 17 shows the DVS isotherm of Example 1 (I-1a; fumarate);

FIG. 18 shows the DVS isotherm of Example 2 (I-1b; benzoate);

FIG. 19 shows the DVS isotherm of Example 4 (I-1d; succinate);

FIG. 20 shows the DVS isotherm of Example 3 (I-1c; salicylate);

FIG. 21 shows the DVS isotherm of Example 5 (I-1e; oxalate);

FIG. 22 shows the DVS isotherm of Example 6 (I-1f; glycolate);

FIG. 23 shows the DVS isotherm of Example 8 (I-1h; hemi-fumarate);

FIG. 24 shows the DVS isotherm of Example 7 (I-1g; hemi-oxalate);

FIG. 25 shows a ¹H NMR spectra of Example 1 (I-1a; fumarate);

FIG. 26 shows a ¹H NMR spectra of Example 2 (I-1b; benzoate);

FIG. 27 shows a ¹H NMR spectra of Example 3 (I-1c; salicylate);

FIG. 28 shows a ¹H NMR spectra of Example 5 (I-1e; oxalate);

FIG. 29 shows a ¹H NMR spectra of Example 8 (I-1h; hemi-fumarate);

FIG. 30 shows a ¹H NMR spectra of Example 6 (I-1f; glycolate);

FIG. 31 shows a ¹H NMR spectra of Example 4 (I-1d; succinate);

FIG. 32 shows a ¹H NMR spectra of Example 7 (1-1g; hemi-oxalate); and

FIGS. 33A-33B show a single crystal of I-8b (benzoate) in terms of molecular structure (FIG. 33A) and asymmetric crystal unit cell (FIG. 33B).

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

When it is stated that a substituent or group “comprise(s) deuterium” or is “comprising deuterium,” it is to be understood that the substituent or group may itself be deuterium, or the substituent or group may contain at least one deuterium substitution in its chemical structure. For example, when substituent “-R” is defined to comprise deuterium, it is to be understood that -R may be -D (-deuterium), or a group such as —CD₃ that is consistent with the other requirements set forth of -R.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

A “crystalline” solid is a type of solid whose fundamental three-dimensional structure contains a highly regular pattern of atoms or molecules—with long range order-forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity-less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of a known standard or an internal standard. Other characterization techniques, such as differential scanning calorimetry (DSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

When referencing X-ray powder diffraction (XRPD) patterns of materials of the present disclosure, the phrase “characterized by an X-ray powder diffraction pattern containing at least three characteristic peaks at diffraction angles (2θ±0.2°) selected from . . . ” should be understood to include those materials characterized as having 3, 4, 5, 6, 7, 8, 9, 10, 11, 12, 13, 14, 15 or more (including all) of the recited characteristic XRPD diffraction peaks. Further, this phrase is intended to be open to the inclusion of other XRPD diffraction peaks not recited.

It will be appreciated that the compounds herein can exist in different salt, solvate, stereoisomer, crystalline/amorphous (including polymorphic) forms, and the present disclosure is intended to include all permutations thereof, such as a solvate of a pharmaceutically acceptable salt of a stereoisomer of the subject compound.

A “vapor” is a solid substance in the gas phase at a temperature lower than its critical temperature, meaning that the vapor can be condensed to a liquid by increasing the pressure on it without reducing the temperature.

An “aerosol”, as used herein, is a suspension of fine solid particles or liquid droplets in a gas phase (e.g., air, oxygen, helium, nitrous oxide, and other gases, as well as mixtures thereof). A “mist”, as used herein, is a subset of aerosols, differing from a vapor, and is a dispersion of liquid droplets (liquid phase) suspended in the gas phase (e.g., air, oxygen, helium, and mixtures thereof). The liquid droplets of an aerosol or mist can comprise a drug moiety dissolved in an aqueous liquid, organic solvent, or a mixture thereof. The gas phase of an aerosol or mist can comprise air, oxygen, helium, or other gases, including mixtures thereof. Mists do not comprise solid particulates. Aerosols and mists of the present disclosure can be generated by any suitable methods and devices, examples of which are set forth herein, e.g., through use of an inhaler or nebulizer.

As used herein, the language “sustained release” describes the release period for certain formulations of the present disclosure formulated to increase the release period e.g., to a maximum value, which in the case of ingested oral formulations is ultimately limited by the time the gastrointestinal tract naturally excretes all drugs with food. As used herein, the language “release period” describes the time window in which any compound described herein is released from the vehicle (e.g., matrix) to afford plasma concentrations of compounds described herein. The start time of the release period is defined from the point of administration to a subject, which when ingested orally is considered nearly equivalent to entry into the stomach, and initial dissolution by gastric enzymes and acid. The end time of the release period is defined as the point when the entire loaded drug is released. In some embodiments, the release period can be greater than about 4 hours, 8 hours, 12 hours, 16 hours, or 20 hours, greater than or equal to about 24 hours, 28 hours, 32 hours, 36 hours, or 48 hours, or less than about 48 hours, 36 hours, 4 hours or less, 3 hours or less, 2 hours or less, or 1 hour or less.

The language “tamper resistant” is art-recognized to describe aspects of a drug formulation that make it more difficult to use the formulation to abuse the drug moiety of the formulation through e.g., extraction for intravenous use, or crushing for freebase use; and therefore, reduce the risk for abuse of the drug.

The term “stable,” “stability,” and the like, as used herein includes chemical stability and solid state (physical) stability. The term “chemical stability” means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g., hydration, dehydration, solvatization, desolvatization, crystallization, recrystallization or solid-state phase transition).

As used herein, the term “composition” is equivalent to the term “formulation.” As used herein, the term “inhalation session” describes a dosing event whereby the subject inhales a given dose of drug, irrespective of the number of breadths needed to inhale the given dose. For example, a subject prescribed to take 10 mg of a drug twice a day would undertake two inhalation sessions, each inhalation session providing 10 mg of the drug. The length of time and the number of breaths for each inhalation session would be dependent on factors such as the inhalation device used, the amount of drug that is drawn per breath, the concentration of the drug in the dosage form, the subject's breathing pattern, etc.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating one or more symptoms of the disease or medical condition in a patient. A treatment can provide a therapeutic benefit such as the eradication or amelioration of one or more of the physiological or psychological symptoms associated with the underlying condition, disease, or disorder such that an improvement is observed in the patient, notwithstanding the fact that the patient may still be affected by the condition. In some embodiments, treatment may refer to prophylaxis, i.e., preventing the disease or medical condition from occurring or otherwise delaying the onset of the disease or medical condition in a patient.

A “patient” or “subject,” used interchangeably herein, can be any mammal including, for example, a human. A patient or subject can have a condition to be treated or can be susceptible to a condition to be treated.

As used herein, and unless otherwise specified, the terms “manage,” “managing” and “management” refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the term “managing” encompasses treating a subject who had suffered from the particular disease, disorder, or condition in an attempt to prevent or minimize the recurrence of the disease, disorder, or condition, or of one or more symptoms thereof.

“Therapeutically effective amount” refers to an amount of a compound(s) or its salt form sufficient to treat a specified disorder or disease or one or more of its symptoms and/or to prevent the occurrence of the disease or disorder (prophylactically effective amount). As used herein, and unless otherwise specified, a “prophylactically effective amount” of an active agent, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The language “toxic spikes” is used herein to describe spikes in concentration of any compound described herein that would produce side-effects of sedation or psychotomimetic effects, e.g., hallucination, dizziness, and nausea; which can not only have immediate repercussions, but also influence treatment compliance. In particular, side effects may become more pronounced at blood concentration levels above about 300 ng/L (e.g. above about 300, 400, 500, 600 or more ng/L).

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, schizophrenia, cognitive deficits in schizophrenia, attention deficit disorder, attention deficit hyperactivity disorder, bipolar and manic disorders, depression or any combinations thereof.

“Inflammatory conditions” or “inflammatory disease,” as used herein, refers broadly to chronic or acute inflammatory diseases. Inflammatory conditions and inflammatory diseases include, but are not limited to, rheumatic diseases (e.g., rheumatoid arthritis, osteoarthritis, psoriatic arthritis); spondyloarthropathies (e.g., ankylosing spondylitis, reactive arthritis, Reiter's syndrome); crystal arthropathies (e.g., gout, pseudogout, calcium pyrophosphate deposition disease); multiple sclerosis; Lyme disease; polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, glaucoma, and cataracts.

All diseases and disorders listed herein may be defined as described in the Diagnostic and Statistical Manual of Mental Disorders (DSM-5), published by the American Psychiatric Association, or in International Classification of Diseases (ICD), published by the World Health Organization.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value may vary up or down by 5%. For example, for a value of about 100, means 95 to 105 (or any value between 95 and 105).

### Compounds and Salt Forms

Disclosed herein is a pharmaceutically acceptable salt of a compound of Formula (I), or a stereoisomer, solvate, or prodrug thereof,

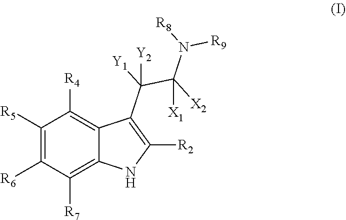

wherein:
X₁ and X₂ are independently hydrogen or deuterium,
Y₁ and Y₂ are independently hydrogen or deuterium,
R₂, R₄, R₅, R₆, and R₇ are independently hydrogen or deuterium, and R₈ and R₉ are independently selected from the group consisting of —CH₃, —CDH₂, —CD₂H, and —CD₃.

X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ is deuterium and X₂ is hydrogen.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ is deuterium and Y₂ is hydrogen.

In some embodiments, X₁, X₂, Y₁, and Y₂ are hydrogen. In some embodiments, X₁, X₂, Y₁, and Y₂ are deuterium.

In some embodiments, R₂ is deuterium. In some embodiments, R₂ is hydrogen.

In some embodiments, R₄ is deuterium. In some embodiments, R₄ is hydrogen.

In some embodiments, R₅ is deuterium. In some embodiments, R₅ is hydrogen.

In some embodiments, R₆ is deuterium. In some embodiments, R₆ is hydrogen.

In some embodiments, R₇ is deuterium. In some embodiments, R₇ is hydrogen.

R₂, R₄, R₅, R₆, and R₇ may be the same, for example, R₂, R₄, R₅, R₆, and R₇ may each be hydrogen, or alternatively, R₂, R₄, R₅, R₆, and R₇ may each be deuterium. In some embodiments, at least one of R₂, R₄, R₅, R₆, and R₇ is deuterium, or at least two of R₂, R₄, R₅, R₆, and R₇ are deuterium, or at least three of R₂, R₄, R₅, R₆, and R₇ are deuterium, or at least four of R₂, R₄, R₅, R₆, and R₇ are deuterium.

R₈ and R₉ may be the same, or different. In some embodiments, R₈ and R₉ are the same. In some embodiments, R₈ and R₉ are methyl (—CH₃). In some embodiments, R₈ and R₉ are a partially deuterated methyl group, i.e., —CDH₂ or —CD₂H. In some embodiments, R₈ and R₉ are a fully deuterated methyl group (—CD₃).

In some embodiments, at least one of X₁, X₂, Y₁, Y₂, R₂, R₄, R₅, R₆, R₇, R₈, and R₉ comprises deuterium. In some embodiments, at least X₁, X₂, R₈, and R₉ comprise deuterium. In some embodiments, at least X₁, X₂, Y₁, Y₂, R₈, and R₉ comprise deuterium. In some embodiments, X₁, X₂, Y₁, and Y₂ are deuterium, and R₈ and R₉ are a fully deuterated methyl group (—CD₃).

Without being bound to any particular theory, it is believed that the compounds described herein having site-specific deuteration, like in the exocyclic moiety, maintain preferential agonism of serotonin 5-HT₂ receptors, have improved exposure (e.g., prevent high drug concentrations (spiking) observed acutely after administration), and possess advantageous enzymatic degradation profiles for improved bioavailability and brain penetration. For example, the compounds of Formula (I) containing deuterium substitution may advantageously slow enzymatic degradation compared to compounds which may otherwise be susceptible to MAO mediated deamination/oxidation processes to e.g., the indole acetic acid (IAA) metabolite, thereby improving bioavailability as well as enhancing brain levels of the active compound, with the objective to effectively reduce therapeutic doses and to prevent high drug concentrations (“spiking”) observed acutely after administration. As a result, such compounds may result in reduced side effects and toxicity, including toxicity caused by activation of 5-HT_{2B} receptors associated with valvular heart disease.

The compounds of Formula (I) may contain a stereogenic center. In such cases, the compounds may exist as different stereoisomeric forms, even though Formula (I) is drawn without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

In some embodiments, the compounds described herein, e.g., compounds of Formula (I), are non-stereogenic. In some embodiments, the compounds described herein, e.g., compounds of Formula (I), are racemic. In some embodiments, the compounds described herein, e.g., compounds of Formula (I), are enantiomerically enriched (one enantiomer is present in a higher percentage), including enantiomerically pure. In some embodiments, the compounds described herein, e.g., compounds of Formula (I), are provided as a single diastereomer. In some embodiments, the compounds described herein, e.g., compounds of Formula (I), are provided as a mixture of diastereomers. When provided as a mixture of diastereomers, the mixtures may include equal mixtures, or mixtures which are enriched with a particular diastereomer (one diastereomer is present in a higher percentage than another).

In some embodiments, the compound of Formula (I) is an agonist of a serotonin 5-HT₂ receptor.

In some embodiments, the compound of Formula (I) is an agonist of a serotonin 5-HT_{2A} receptor.

In some embodiments, the compound of Formula (I) is selected from the group consisting of

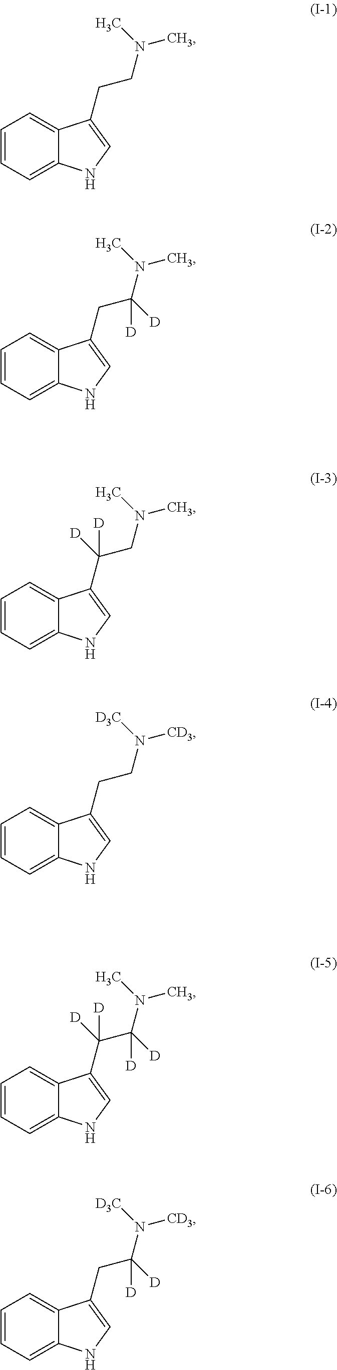 [SMILES: CN(C)CCc1c[nH]c2ccccc12 ; [2H]C([2H])(Cc1c[nH]c2ccccc12)N(C)C ; [2H]C([2H])(CN(C)C)c1c[nH]c2ccccc12 ; [2H]C([2H])([2H])N(CCc1c[nH]c2ccccc12)C([2H])([2H])[2H] ; [2H]C([2H])(c1c[nH]c2ccccc12)C([2H])([2H])N(C)C ; [2H]C([2H])([2H])N(C([2H])([2H])[2H])C([2H])([2H])Cc1c[nH]c2ccccc12]

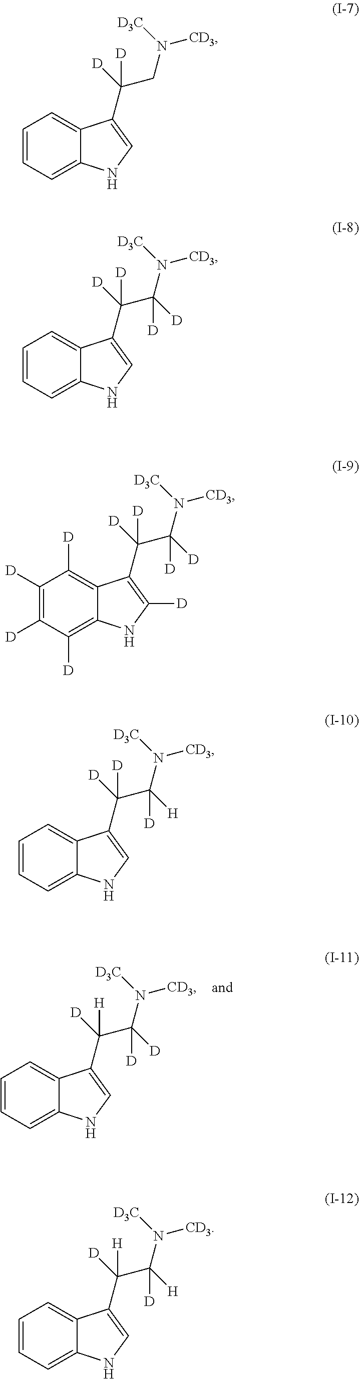 [SMILES: [2H]C([2H])(CN(C([2H])([2H])[2H])C([2H])([2H])[2H])c1c[nH]c2ccccc12 ; [2H]C([2H])([2H])N(C([2H])([2H])[2H])C([2H])([2H])C([2H])([2H])c1c[nH]c2ccccc12 ; [2H]c1[nH]c2c([2H])c([2H])c([2H])c([2H])c2c1C([2H])([2H])C([2H])([2H])N(C([2H])([2H])[2H])C([2H])([2H])[2H] ; [2H]C(N(C([2H])([2H])[2H])C([2H])([2H])[2H])C([2H])([2H])c1c[nH]c2ccccc12 ; [2H]C(c1c[nH]c2ccccc12)C([2H])([2H])N(C([2H])([2H])[2H])C([2H])([2H])[2H] ; [2H]C(c1c[nH]c2ccccc12)C([2H])N(C([2H])([2H])[2H])C([2H])([2H])[2H]]

The compound number, IUIPAC name, and substituent listing for the above-identified compounds are provided in Table 1.

TABLE 1
Exemplary compounds of Formula (I)

| | | Formula (I) | | | | | | | |
|---|---|---|---|---|---|---|---|---|---|
| Compound identifier and name | | X₁, X₂ | Y₁, Y₂ | R₂ | R₄ | R₅ | R₆ | R₇ | R₈, R₉ |
| I-1 | 2-(1H-indol-3-yl)-N,N-dimethylethan- | H, H | H, H | H | H | H | H | H | —CH₃, —CH₃ |
| | 1-amine | | | | | | | | |
| I-2 | 2-(1H-indol-3-yl)-N,N-dimethylethan- | D, D | H, H | H | H | H | H | H | —CH₃, —CH₃ |
| | 1-amine-1,1-d₂ | | | | | | | | |
| I-3 | 2-(1H-indol-3-yl)-N,N-dimethylethan- | H, H | D, D | H | H | H | H | H | —CH₃, —CH₃ |
| | 1-amine-2,2-d₂ | | | | | | | | |
| I-4 | 2-(1H-indol-3-yl)-N,N-bis(methyl- | H, H | H, H | H | H | H | H | H | —CD₃, —CD₃ |
| | d₃)ethan-1-amine | | | | | | | | |
| I-5 | 2-(1H-indol-3-yl)-N,N-dimethylethan- | D, D | D, D | H | H | H | H | H | —CH₃, —CH₃ |
| | 1-amine-1,1,2,2-d₄ | | | | | | | | |
| I-6 | 2-(1H-indol-3-yl)-N,N-bis(methyl- | D, D | H, H | H | H | H | H | H | —CD₃, —CD₃ |
| | d₃)ethan-1-amine-1,1-d₂ | | | | | | | | |
| I-7 | 2-(1H-indol-3-yl)-N,N-bis(methyl- | H, H | D, D | H | H | H | H | H | —CD₃, —CD₃ |
| | d₃)ethan-1-amine-2,2-d₂ | | | | | | | | |
| I-8 | 2-(1H-indol-3-yl)-N,N-bis(methyl- | D, D | D, D | H | H | H | H | H | —CD₃, —CD₃ |
| | d₃)ethan-1-amine-1,1,2,2-d₄ | | | | | | | | |
| I-9 | 2-(1H-indol-3-yl-2,4,5,6,7-d₅)-N,N- | D, D | D, D | D | D | D | D | D | —CD₃, —CD₃ |
| | bis(methyl-d₃)ethan-1-amine-1,1,2,2-d₄ | | | | | | | | |
| I-10 | 2-(1H-indol-3-yl)-N,N-bis(methyl- | D, H | D, D | H | H | H | H | H | —CD₃, —CD₃ |
| | d₃)ethan-1-amine-1,2,2-d₃ | | | | | | | | |
| I-11 | 2-(1H-indol-3-yl)-N,N-bis(methyl- | D, D | D, H | H | H | H | H | H | —CD₃, —CD₃ |
| | d₃)ethan-1-amine-1,1,2-d₃ | | | | | | | | |
| I-12 | 2-(1H-indol-3-yl)-N,N-bis(methyl- | D, H | D, H | H | H | H | H | H | —CD₃, —CD₃ |
| | d₃)ethan-1-amine-1,2-d₂ | | | | | | | | |

Acids for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, a-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

Certain salts are preferred among the list above because they possess physical and pharmaceutical characteristics/properties which make them suitable for pharmaceutical preparation and administration. For example, preferred salt forms of the compounds disclosed herein (e.g., compounds of Formula (I)) are those that possess one or more of the following characteristics: are easy to prepare in high yield with a propensity towards salt formation; are stable and have well-defined physical properties such as crystallinity, lack of polymorphism, and high melting/enthalpy of fusion; have slight or no hygroscopicity; are free flowing, do not cohere/adhere to surfaces, and possess a regular morphology; have acceptable aqueous solubility for the intended route of administration; and/or are physiologically acceptable, particularly for pulmonary administration, e.g., do not cause irritation when administered into the lungs.

### Crystallinity

The pharmaceutically acceptable salt of the compound of Formula (I) may be crystalline or amorphous, preferably crystalline, as determined e.g., by X-ray powder diffraction (XRPD). In some embodiments, the pharmaceutically acceptable salt of the compound of Formula (I) is amorphous. Amorphous forms typically possess higher aqueous solubility and rates of dissolution compared to their crystalline counterparts, and thus may be well suited for quick acting dosage forms adapted to rapidly release the active ingredient, such as orodispersible dosage forms, immediate release (IR) dosage forms, and the like. The pharmaceutically acceptable salt of the compound of Formula (I) can be in a stable amorphous form. In some embodiments, the pharmaceutically acceptable salt of the compound of Formula (I) is provided in amorphous form, e.g., as determined by XRPD and/or DSC. Accordingly, pharmaceutical compositions may be prepared from pharmaceutically acceptable salt forms of compounds of Formula (I), in one or more amorphic forms, and may be used for treatment as set forth herein. In some embodiments, a highly pure amorphous form of a pharmaceutically acceptable salt of a compound of Formula (I) is provided. For example, the pharmaceutical composition may comprise a pharmaceutically acceptable salt of a compound of Formula (I), wherein at least 92%, at least 94%, at least 96%, at least 98%, at least 99%, or at least 99.5% by weight of the pharmaceutically acceptable salt of the compound of Formula (I) present in the pharmaceutical composition is in amorphous form, e.g., as determined by X-ray powder diffraction and/or DSC.

In some embodiments, the pharmaceutically acceptable salt of the compound of Formula (I) is crystalline. Crystalline forms are advantageous in terms of e.g., stability and providing well-defined physical properties, which is desirable for pharmaceutical preparation and administration. The pharmaceutically acceptable salts of the compound of Formula (I) can be in a stable crystalline form. In some embodiments, the pharmaceutically acceptable salt of the compound of Formula (I) has a percent crystallinity of at least 50%, at least 60%, at least 70%, at least 80%, at least 90%, at least 95%, at least 99%, or at least 99.5%, and up to 100%, as determined by XRPD and/or DSC analysis. In some embodiments, a highly pure crystalline form of a pharmaceutically acceptable salt of a compound of Formula (I) is provided. For example, the pharmaceutical composition may comprise a pharmaceutically acceptable salt of a compound of Formula (I), wherein at least 90%, at least 95%, at least 99%, or at least 99.5% by weight of the pharmaceutically acceptable salt of the compound of Formula (I) present in the pharmaceutical composition is in crystalline form, e.g., as determined by X-ray powder diffraction and/or DSC. Preference is given to salt forms with high crystallinity, as determined e.g., by discrete and sharp Bragg diffractions in the X-ray diffractograms.

XRPD analyses can be carried out, e.g., on a Bruker D5000 X-ray powder diffractometer using CuKα radiation (wavelength=1.54060 Å). The instrument may be equipped with a fine focus X-ray tube. The tube voltage and amperage can be set to 40 kV and 30 mA, respectively. The divergence and scattering slit widths can be set at 2 mm and the detector slit width can be set at 0.2 mm. Diffracted radiation can be detected by a NaI scintillation detector. A theta-two theta continuous scan from 2.0 to 40° (4 seconds per step; 0.01° step size) can be used.

In terms of pharmaceutical production processes, advantageous salt forms of the compounds of Formula (I) are those that readily afford a crystalline solid on crystallization in acceptable yield without proceeding via an oil, and with favorable volume factors, making them suitable for mass production.

Salts forms of the compound of Formula (I) can exist in different polymorphs (i.e., forms having a different crystal structure), however, preferred salt forms of the present disclosure are those which can be crystallized into a single crystalline form or single polymorph, as determined by XRPD and/or differential scanning calorimetry (DSC). It is also generally desirable for the salts to be free flowing, not cohere/adhere to surfaces, and possess a regular morphology.

Chemical/Solid-state Stability In some embodiments, the pharmaceutically acceptable salt of the compound of Formula (I) has a melt onset of from about 100° C., from about 110° C., from about 120° C., from about 130° C., from about 140° C., from about 150° C., from about 160° C., from about 170° C., from about 180° C., from about 190° C., and up to about 250° C., up to about 225° C., up to about 210° C., up to about 200° C., as determined by DSC.

In some embodiments, the pharmaceutically acceptable salt of the compound of Formula (I) has an enthalpy of fusion of from about 90 J·g⁻¹, from about 100 J·g⁻¹, from about 110 J·g⁻¹, from about 120 J·g⁻¹, from about 130 J·g⁻¹, from about 140 J·g⁻¹, from about 150 J·g⁻¹, from about 160 J·g⁻¹, and up to about 190 J·g⁻¹, up to about 180 J·g⁻¹, up to about 170 J·g⁻¹, as determined by DSC.

Pharmaceutically acceptable salts of the compound of Formula (I) suitable for pharmaceutical manufacture may also be characterized as non-hygroscopic or slightly hygroscopic, preferably non-hygroscopic. The hygroscopicity may be measured herein by performing a moisture adsorption-desorption isotherm using a dynamic vapor sorption (DVS) analyzer with a starting exposure of 30% relative humidity (RH), increasing humidity up to 95% RH, decreasing humidity to 0%, and finally increasing the humidity back to the starting 30% RH, and classified according to the following:
non-hygroscopic: <0.2%; slightly hygroscopic: ≥0.2% and <2%; hygroscopic: ≥2% and <15%; very hygroscopic: ≥15%; deliquescent: sufficient water is absorbed to form a liquid; all values measured as weight increase (w/w due to acquisition of water) at >95% RH and 25° C.

In some embodiments, the pharmaceutically acceptable salt of the compound of Formula (I) has a weight increase at >95% RH of less than 1% w/w, less than 0.8% w/w, less than 0.6% w/w, less than 0.5% w/w, less than 0.4% w/w, less than 0.3% w/w, less than 0.2% w/w, less than 0.1% w/w, less than 0.08% w/w, less than 0.06% w/w, less than 0.05% w/w, less than 0.02% w/w, as determined by DVS.

Dry powder samples of the pharmaceutically acceptable salts of the present disclosure can be maintained/stored in open or closed environments, such as in open or closed flasks/vials, under ambient or stress conditions e.g., 25° C./60% RH, 25° C./90+% RH, 40° C./75% RH, etc. without appreciable degradation (e.g., without appreciably diminished chemical purity) or physical changes (e.g., changed forms, deliquesced, etc.). For example, dry powder samples of salt forms disclosed herein may have a purity or form change of less than 10%, less than 5%, less than 1%, when stored under ambient conditions or stress conditions (e.g., increased temperature, e.g., 40° C., and/or humidity).

Solution-phase compositions (e.g., liquid dosage forms) of the pharmaceutically acceptable salts of the present disclosure can be maintained/stored in open or closed environments, such as in open or closed flasks/vials, under ambient or stress conditions e.g., 25° C./90+% RH, 40° C./75% RH, etc. without appreciable degradation. Thus, in some embodiments, the present disclosure provides stable solution-phase compositions (e.g., liquid dosage forms) of pharmaceutically acceptable salt forms of the compounds of Formula (I) (e.g., stable solvates of salt forms of compounds of Formula (I) which are in solvated form, preferably fully solvated form), which can be stored as a solution, such as in the form of an aqueous solution, an organic solvent solution, or a mixed aqueous-organic solvent solution, for prolonged periods of time without appreciable degradation or physical changes, such as oiling out of solution. Solvents which can be used to form the solution-phase compositions can be any one or more solvents set forth herein, e.g., water, ethanol, fruit juice, etc. In some embodiments, the solution-phase composition is an aqueous solution-phase composition comprising a pharmaceutically acceptable salt of the compound of Formula (I) solvated with water (and optionally comprising other components such as those found in fruit juice). The identification of stable solution-phase compositions is advantageous at least because such compositions do not require use immediately after being prepared, such as within 5 minutes, within 4 minutes, within 3 minutes, within 2 minutes, within 1 minute, within 45 seconds, within 30 seconds, within 15 seconds, within 10 seconds of being prepared. Instead, the stable solution-phase compositions (e.g., liquid dosage forms) of the pharmaceutically acceptable salts of the compounds of Formula (I) described herein can be prepared in advance, when desired, optionally stored, and can be administered hours, days, or even weeks after being prepared, without materially effecting efficacy, e.g., without appreciable degradation of the dimethyltryptamine-type active.

In some embodiments, aqueous solutions formed from the pharmaceutically acceptable salt of the compound of Formula (I) are characterized by increased stability compared to aqueous solutions that are prepared from the compound of Formula (I) (free base) but are otherwise substantially the same. For example, the pharmaceutically acceptable salt of the compound of Formula (I) may be at least 5%, at least 10%, at least 15%, at least 20%, at least 25%, at least 30%, at least 35%, at least 40%, at least 45%, at least 50%, at least 55%, at least 60%, at least 65%, at least 70% more stable in aqueous solution subjected to 40° C. for 24 hours, 48 hours, 72 hours, 1 week, 2 weeks, 3 weeks, 4 weeks, or longer, in terms of chemical purity (% active remaining), compared to aqueous solutions prepared with the compound of Formula (I) (free base) but are otherwise substantially the same. Such improved stability behavior can also be found in pharmaceutical compositions of the present disclosure.

### Physiologically Acceptability

Suitable salt forms of the compounds of Formula (I) are physiologically acceptable. In particular, when formulated for pulmonary administration, suitable salt forms are those which do not cause excessive irritation, as lung irritation may lead to post-inhalation coughing, which may adversely affect patient compliance as well as treatment efficacy since coughing is known to reduce the delivered dose. For this reason, preferred addition salts of the compound of Formula (I) are those formed with an organic acid, preferably an organic acid with a mild acidity, for example an organic acid with a pKₐ in water of no less than 1.0, no less than 1.5, no less than 2.0, no less than 2.5, no less than 3.0, no less than 3.5, no less than 4.0, no less than 4.5, for example, from 3.0 to 6.5. Further, it may also be desirable to use acid addition salts that impart a pleasant taste profile (e.g., sweet, citrus flavored, etc.), although poor tasting salt forms (e.g., bitter, harsh, etc.) may still be acceptable depending on, for example, the route of administration and the optional use of taste masking agents such as sweetening agents, flavoring agents, etc.

### Solubility

The aqueous solubility of compounds of Formula (I) and their salts can be determined by equilibrating excess solid with 1 mL of water for 24 hours at 22° C. A 200 μL aliquot can be centrifuged at 15,000 rpm for 15 minutes. The supernatant can be analyzed by HPLC and the solubility can be expressed as its free base equivalent (mg FB/mL). For example, pharmaceutically acceptable salts of compound of Formula (I) can be prepared and the solubility and solution pH can be measured.

In some embodiments, the pharmaceutically acceptable salt of the compound of Formula (I) has a water solubility at 22° C. of from about 5 mg/mL to about 400 mg/mL. In some embodiments, the pharmaceutically acceptable salt of the compound of Formula (I) has a water solubility of from about 1 mg/mL, from about 2 mg/mL, from about 3 mg/mL, from about 5 mg/mL, from about 10 mg/mL, from about 20 mg/mL, from about 30 mg/mL, from about 40 mg/mL, from about 50 mg/mL, from about 60 mg/mL, from about 70 mg/mL, from about 80 mg/mL, from about 90 mg/mL, from about 100 mg/mL, from about 110 mg/mL, from about 120 mg/mL, from about 130 mg/mL, from about 140 mg/mL, from about 150 mg/mL, and up to about 400 mg/mL, up to about 380 mg/mL, up to about 360 mg/mL, up to about 340 mg/mL, up to about 320 mg/mL, up to about 300 mg/mL, up to about 280 mg/mL, up to about 260 mg/mL, up to about 250 mg/mL. In some embodiments, the salt of the compound of Formula (I) has a water solubility from about 200 mg/mL to about 400 mg/mL. In some embodiments, the salt of the compound of Formula (I) has a water solubility from about 150 mg/mL to about 250 mg/mL. In some embodiments, the salt of the compound of Formula (I) has a water solubility of greater than about 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL.

In some embodiments, the pharmaceutically acceptable salt of the compound of Formula (I) is a fumarate, a benzoate, a salicylate, a succinate, an oxalate, a glycolate, a hemi-oxalate, or a hemi-fumarate salt of the compound of Formula (I). In terms of providing desirable physical and pharmaceutical characteristics, such as those described above, preferred pharmaceutically acceptable salts are fumarate salts, benzoate salts, salicylates, and succinate salts of the compounds disclosed herein, e.g., the compounds of Formula (I), with fumarate, benzoate, and salicylate salts being particularly preferred.

Exemplary pharmaceutically acceptable salt forms (i.e., addition salt forms) of the above-identified compounds are provided in Table 2.

TABLE 2
Exemplary pharmaceutically acceptable
salts of compounds of Formula (I)

| Salt form identifier | Salt type of compound |
|---|---|
| I-1a | Fumarate of I-1 |
| I-1b | Benzoate of I-1 |
| I-1c | Salicylate of I-1 |
| I-1d | Succinate of I-1 |
| I-2a | Fumarate of I-2 |
| I-2b | Benzoate of I-2 |
| I-2c | Salicylate of I-2 |
| I-2d | Succinate of I-2 |
| I-3a | Fumarate of I-3 |
| I-3b | Benzoate of I-3 |
| I-3c | Salicylate of I-3 |
| I-3d | Succinate of I-3 |
| I-4a | Fumarate of I-4 |
| I-4b | Benzoate of I-4 |
| I-4c | Salicylate of I-4 |
| I-4d | Succinate of I-4 |
| I-5a | Fumarate of I-5 |
| I-5b | Benzoate of I-5 |
| I-5c | Salicylate of I-5 |
| I-5d | Succinate of I-5 |
| I-6a | Fumarate of I-6 |
| I-6b | Benzoate of I-6 |
| I-6c | Salicylate of I-6 |
| I-6d | Succinate of I-6 |
| I-7a | Fumarate of I-7 |
| I-7b | Benzoate of I-7 |
| I-7c | Salicylate of I-7 |
| I-7d | Succinate of I-7 |
| I-8a | Fumarate of I-8 |
| I-8b | Benzoate of I-8 |
| I-8c | Salicylate of I-8 |
| I-8d | Succinate of I-8 |
| I-9a | Fumarate of I-9 |
| I-9b | Benzoate of I-9 |
| I-9c | Salicylate of I-9 |
| I-9d | Succinate of I-9 |
| I-10a | Fumarate of I-10 |
| I-10b | Benzoate of I-10 |
| I-10c | Salicylate of I-10 |
| I-10d | Succinate of I-10 |
| I-11a | Fumarate of I-11 |
| I-11b | Benzoate of I-11 |
| I-11c | Salicylate of I-11 |
| I-11d | Succinate of I-11 |
| I-12a | Fumarate of I-12 |
| I-12b | Benzoate of I-12 |
| I-12c | Salicylate of I-12 |
| I-12d | Succinate of I-12 |

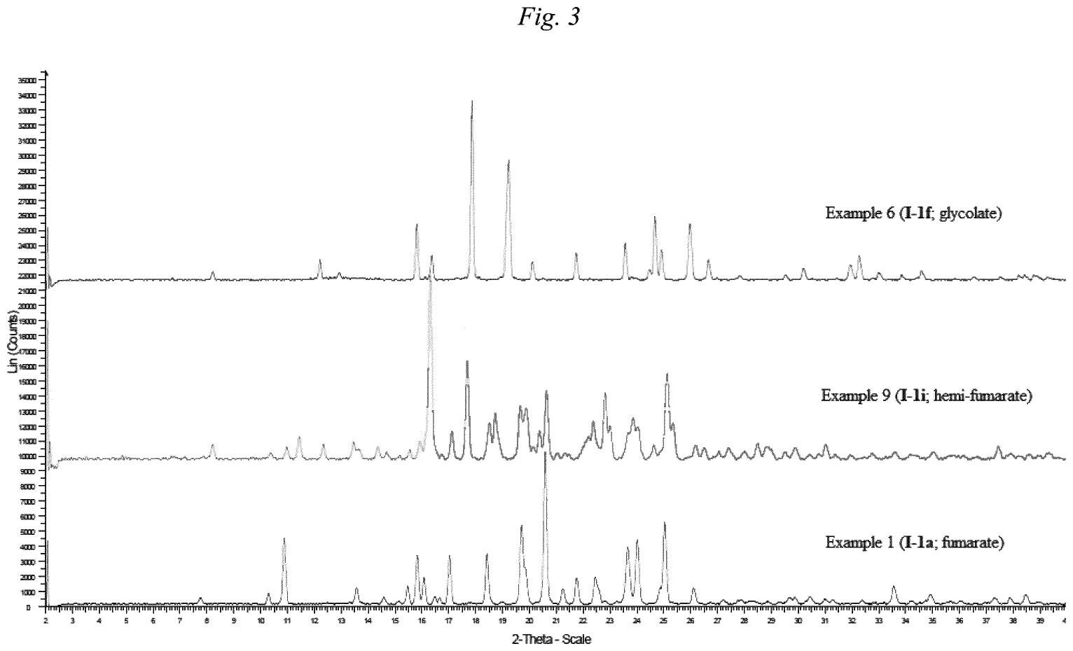

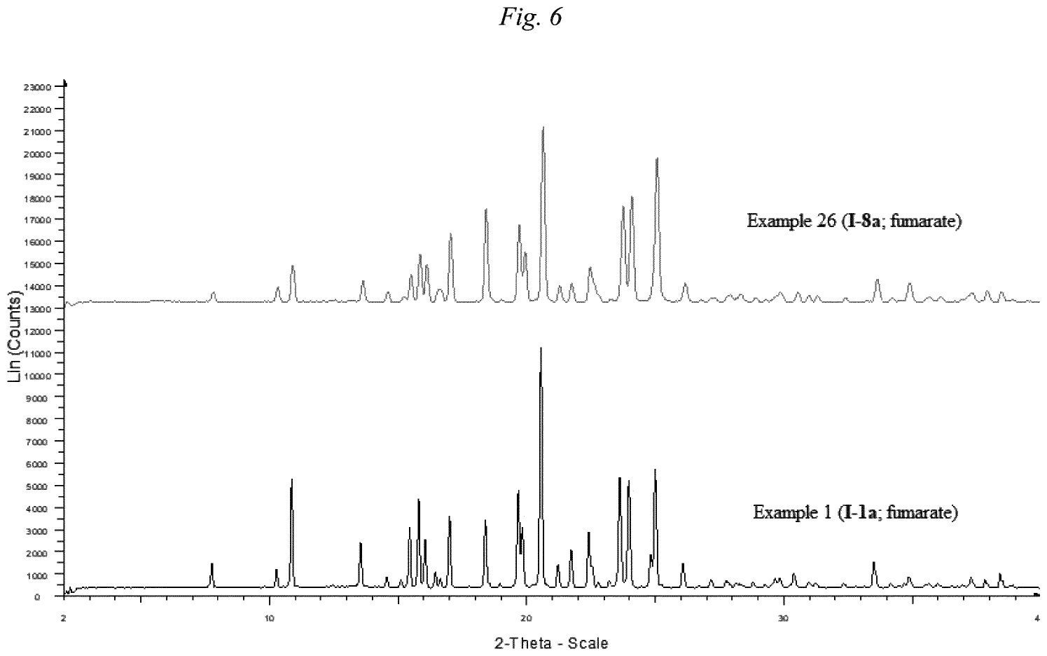

In some embodiments, the pharmaceutically acceptable salt is a fumarate salt of 2-(1H-indol-3-yl)-N,N-dimethylethan-1-amine (I-1a) (i.e., a fumarate salt of compound I-1 depicted below). In some embodiments, salt I-1a is in a crystalline solid form characterized by an X-ray powder diffraction pattern containing at least three characteristic peaks at diffraction angles (2θ±0.2°) selected from 7.8°, 10.3°, 10.9°, 13.6°, 15.8°, 16.1°, 17.0°, 18.4°, 19.7°, 19.9°, 20.6°, 21.3°, 21.7°, 22.5°, 23.9°, 24.1°, 25.1°, 26.2°, 33.6°, and 34.9°, as determined by XRPD using a CuKα radiation source, for example, as shown in FIGS. 3 and 6.

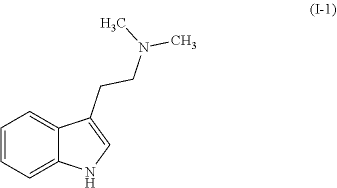

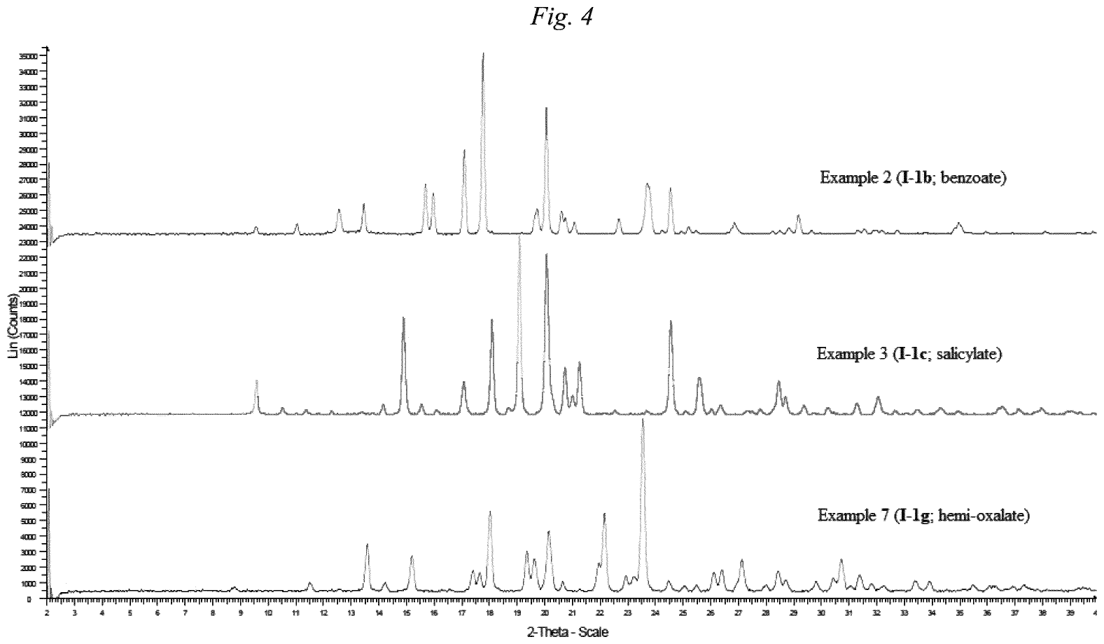

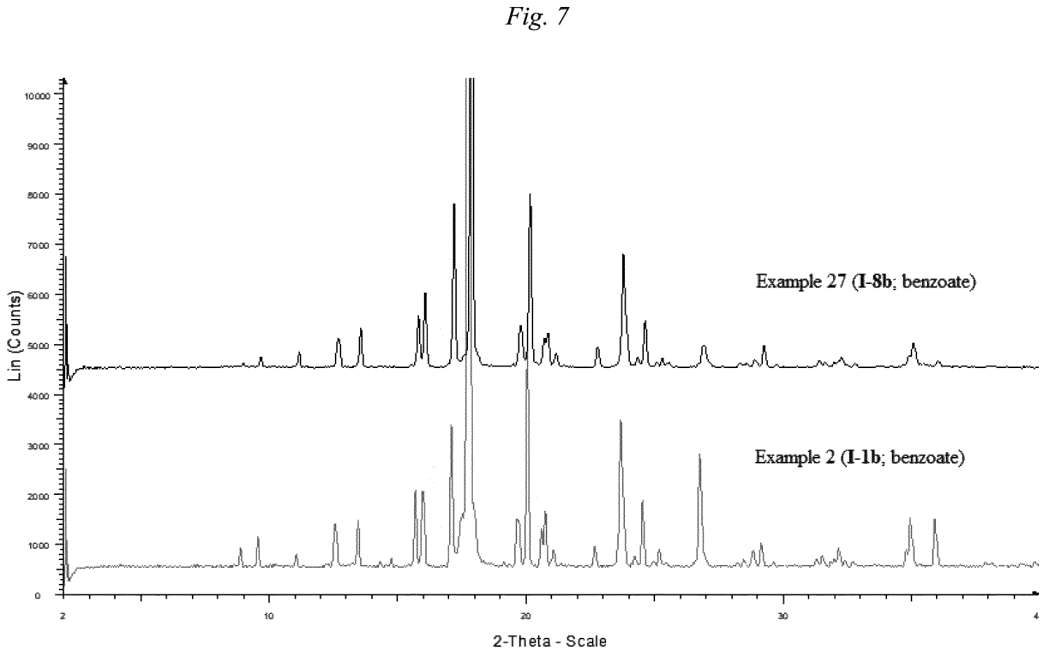

In some embodiments, the pharmaceutically acceptable salt is a benzoate salt of 2-(1H-indol-3-yl)-N,N-dimethylethan-1-amine (I-1b) (i.e., a benzoate salt of compound I-1 depicted above). In some embodiments, salt I-1b is in a crystalline solid form characterized by an X-ray powder diffraction pattern containing at least three characteristic peaks at diffraction angles (2θ±0.2°) selected from 9.6°, 11.1°, 12.6°, 13.5°, 15.8°, 16.1°, 17.1°, 17.9°, 19.8°, 20.1°, 20.8°, 21.2°, 22.7°, 23.8°, 24.6°, 26.9°, 29.2°, 32.3°, 35.1°, and 36.1°, as determined by XRPD using a CuKα radiation source, for example, as shown in FIGS. 4 and 7.

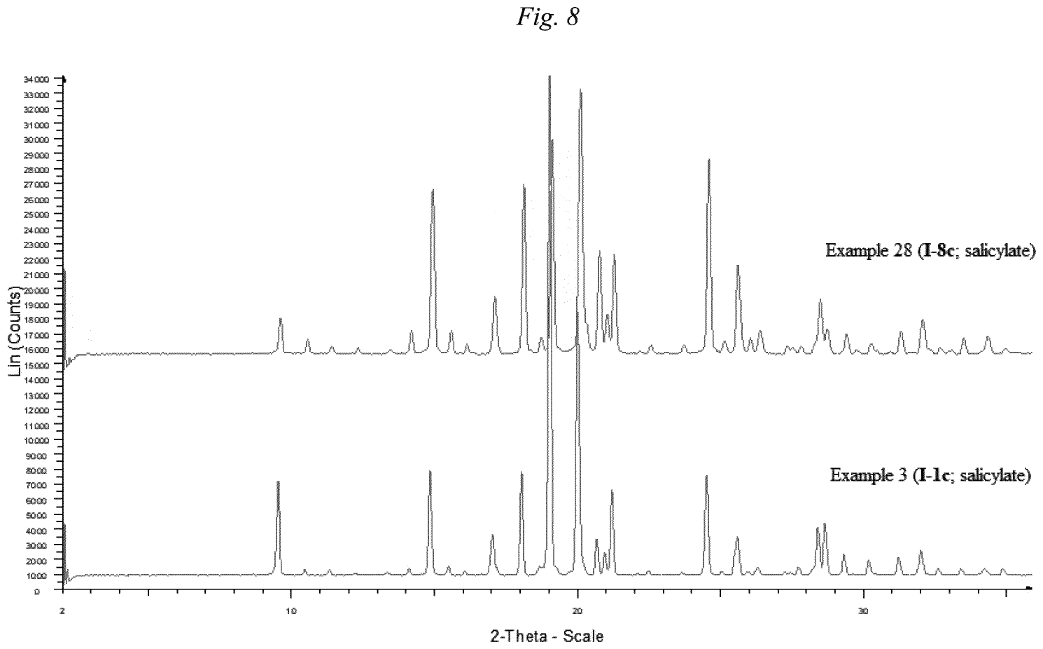

In some embodiments, the pharmaceutically acceptable salt is a salicylate salt of 2-(1H-indol-3-yl)-N,N-dimethylethan-1-amine (I-1c) (i.e., a salicylate salt of compound I-1 depicted above). In some embodiments, salt I-1c is in a crystalline solid form characterized by an X-ray powder diffraction pattern containing at least three characteristic peaks at diffraction angles (2θ±0.2°) selected from 9.6°, 10.5°, 14.9°, 17.1°, 18.1°, 19.1°, 20.1°, 20.7°, 21.0°, 21.3°, 24.6°, 25.6°, 28.5°, 28.8°, 29.4°, 30.3°, 31.3°, 32.10, 33.5°, and 34.4°, as determined by XRPD using a CuKα radiation source, for example, as shown in FIGS. 4 and 8.

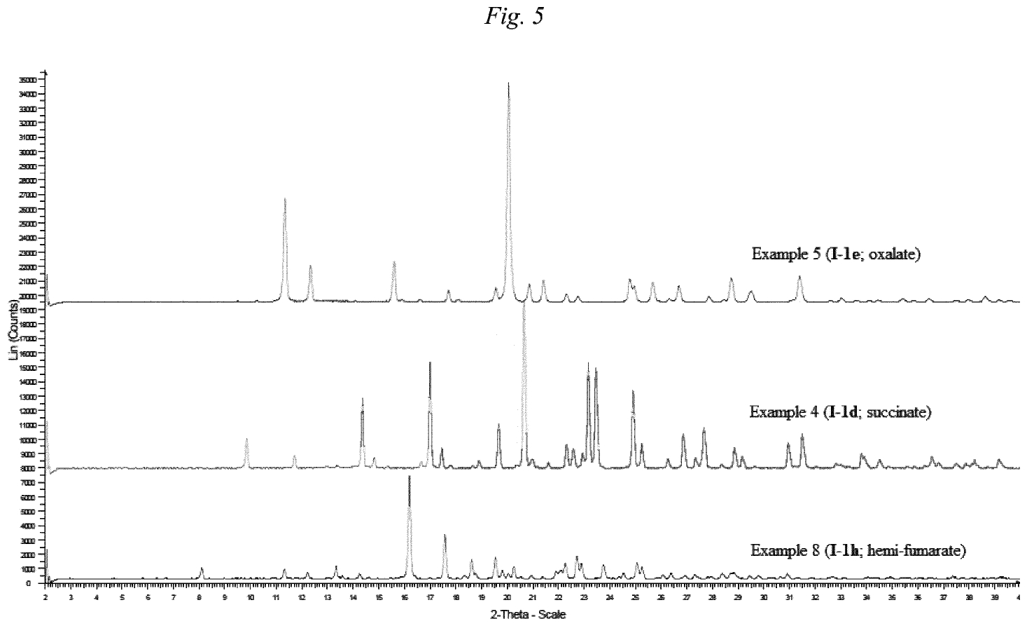

In some embodiments, the pharmaceutically acceptable salt is a succinate salt of 2-(1H-indol-3-yl)-N,N-dimethylethan-1-amine (I-1d) (i.e., a succinate salt of compound I-1 depicted above). In some embodiments, salt I-1d is in a crystalline solid form characterized by an X-ray powder diffraction pattern containing at least three characteristic peaks at diffraction angles (2θ±0.2°) selected from 9.8°, 11.7°, 14.3°, 14.7°, 17.0°, 17.4°, 19.6°, 20.6°, 22.3°, 22.6°, 22.9°, 23.1°, 23.4°, 24.9°, 25.2°, 26.3°, 26.8°, 27.3°, 27.7°, 28.8°, 29.1°, 30.9°, 31.5°, 33.8°, 34.5° 36.5°, and 39.2°, as determined by XRPD using a CuKα radiation source, for example, as shown in FIG. 5.

In some embodiments, the pharmaceutically acceptable salt is an oxalate salt of 2-(1H-indol-3-yl)-N,N-dimethylethan-1-amine (I-1e) (i.e., an oxalate salt of compound I-1 depicted above). In some embodiments, salt I-1e is in a crystalline solid form characterized by an X-ray powder diffraction pattern containing at least three characteristic peaks at diffraction angles (2θ±0.2°) selected from 11.3°, 12.3°, 15.6°, 17.7°, 19.5°, 20.0°, 20.8°, 21.4°, 22.3°, 22.7°, 24.8°, 25.7°, 26.7°, 27.9°, 28.7°, 29.5°, 31.4°, 33.0°, 35.4°, 36.5°, and 38.6°, as determined by XRPD using a CuKα radiation source, for example, as shown in FIG. 5.

In some embodiments, the pharmaceutically acceptable salt is a glycolate salt of 2-(1H-indol-3-yl)-N,N-dimethylethan-1-amine (I-1f) (i.e., a glycolate salt of compound I-1 depicted above). In some embodiments, salt I-1f is in a crystalline solid form characterized by an X-ray powder diffraction pattern containing at least three characteristic peaks at diffraction angles (2θ±0.2°) selected from 8.2°, 12.2°, 12.9°, 15.8°, 16.3°, 17.8°, 19.2°, 20.1°, 21.7°, 23.6°, 24.4°, 24.6°, 24.9°, 26.0°, 26.6°, 27.8°, 29.6°, 30.2°, 32.0°, 32.3°, 33.0°, 33.9° and 34.6°, as determined by XRPD using a CuKα radiation source, for example, as shown in FIG. 3.

In some embodiments, the pharmaceutically acceptable salt is a hemi-oxalate salt of 2-(1H-indol-3-yl)-N,N-dimethylethan-1-amine (I-1g) (i.e., a hemi-oxalate salt of compound I-1 depicted above). In some embodiments, salt I-1g is in a crystalline solid form characterized by an X-ray powder diffraction pattern containing at least three characteristic peaks at diffraction angles (2θ±0.2°) selected from 8.7°, 11.5°, 13.6°, 14.2°, 15.2°, 17.4°, 17.6°, 18.0°, 19.3°, 19.6°, 20.1°, 20.6°, 21.9°, 22.1°, 22.9°, 23.2°, 23.5°, 24.5°, 25.0°, 25.5°, 26.1°, 26.4°, 27.1°, 28.4°, 28.7°, 29.8°, 30.4°, 30.7°, 31.4°, 31.8°, 33.4°, and 33.9°, as determined by XRPD using a CuKα radiation source, for example, as shown in FIG. 4.

In some embodiments, the pharmaceutically acceptable salt is a hemi-fumarate salt of 2-(1H-indol-3-yl)-N,N-dimethylethan-1-amine (I-1h) (i.e., a hemi-fumarate salt of compound I-1 depicted above). In some embodiments, salt I-1h is in a crystalline solid form characterized by an X-ray powder diffraction pattern containing at least three characteristic peaks at diffraction angles (2θ±0.2°) selected from 8.1°, 11.3°, 12.2°, 13.3°, 14.2°, 16.2°, 17.6°, 18.3°, 18.6°, 19.5°, 19.8°, 20.0°, 20.2°, 20.9°, 21.4°, 21.9°, 22.3°, 22.7°, 22.9°, 23.8°, 24.5°, 25.0°, 25.2°, 26.1°, 26.4°, 26.9°, 28.4°, 28.8°, 29.5°, 29.8°, 30.9°, and 32.7°, as determined by XRPD using a CuKα radiation source, for example, as shown in FIG. 5.

In some embodiments, the pharmaceutically acceptable salt is a fumarate salt of 2-(1H-indol-3-yl)-N,N-bis(methyl-d₃)ethan-1-amine-1,1,2,2-d₄ (I-8a) (i.e., a fumarate salt of compound I-8 depicted below). In some embodiments, salt I-8a is in a crystalline solid form characterized by an X-ray powder diffraction pattern containing at least three characteristic peaks at diffraction angles (2θ±0.2°) selected from 7.8°, 10.3°, 10.9°, 12.5°, 13.6°, 14.6°, 15.2°, 15.5°, 15.8°, 16.1°, 16.6°, 17.0°, 18.4°, 19.0°, 19.7°, 19.9°, 20.6°, 21.3°, 21.8°, 22.5°, 23.3°, 23.8°, 24.1°, 25.1°, 26.2°, 26.8°, 27.3°, 27.9°, 28.3°, 28.9°, 29.3°, 29.6°, 29.9°, 30.6°, 31.0°, 31.3°, 32.4°, 32.9°, 33.3°, 33.6°, 34.3°, 34.9°, 35.7°, 36.1°, 37.4°, 38.0°, and 38.5°, as determined by XRPD using a CuKα radiation source, for example, as shown in FIG. 6. In some embodiments, salt I-8a is in a crystalline solid form characterized by an X-ray powder diffraction pattern containing at least three characteristic peaks at diffraction angles (2θ±0.2°) selected from 7.8°, 10.3°, 10.9°, 13.6°, 15.8°, 16.1°, 17.0°, 18.4°, 19.7°, 19.9°, 20.6°, 21.3°, 21.8°, 22.5°, 23.8°, 24.1°, 25.1°, 26.2°, 33.6°, and 34.9° as determined by XRPD using a CuKα radiation source, for example, as shown in FIG. 6. In some embodiments, salt I-8a is in a crystalline solid form characterized by an X-ray powder diffraction pattern containing at least three characteristic peaks at diffraction angles (2θ±0.2°) selected from 10.9°, 13.6°, 15.8°, 16.1°, 17.0°, 18.4°, 19.7°, 19.9°, 20.6°, 23.8°, 24.1°, and 25.1°, as determined by XRPD using a CuKα radiation source, for example, as shown in FIG. 6.

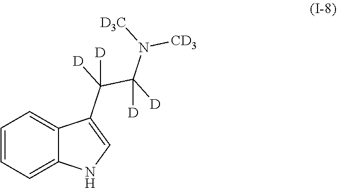 [SMILES: [2H]C([2H])([2H])N(C([2H])([2H])[2H])C([2H])([2H])C([2H])([2H])c1c[nH]c2ccccc12]

In some embodiments, the pharmaceutically acceptable salt is a benzoate salt of 2-(1H-indol-3-yl)-N,N-bis(methyl-d₃)ethan-1-amine-1,1,2,2-d₄ (I-8b) (i.e., a benzoate salt of compound I-8 depicted above). In some embodiments, salt I-8b is in a crystalline solid form characterized by an X-ray powder diffraction pattern containing at least three characteristic peaks at diffraction angles (2θ±0.2°) selected from 9.6°, 11.1°, 12.7°, 13.5°, 15.8°, 16.1°, 17.2°, 17.9°, 19.8°, 20.1°, 20.8°, 21.2°, 22.8°, 23.8°, 24.3°, 24.6°, 25.1°, 25.3°, 25.5°, 26.9°, 28.3°, 28.9°, 29.3°, 31.4°, 31.6°, 32.0°, 32.3°, 32.8°, 35.1°, and 36.1°, as determined by XRPD using a CuKα radiation source, for example, as shown in FIG. 7. In some embodiments, salt I-8b is in a crystalline solid form characterized by an X-ray powder diffraction pattern containing at least three characteristic peaks at diffraction angles (2θ±0.2°) selected from 9.6°, 11.1°, 12.7°, 13.5°, 15.8°, 16.1°, 17.2°, 17.9°, 19.8°, 20.1°, 20.8°, 21.2°, 22.8°, 23.8°, 24.6°, 26.9°, 29.3°, 32.3°, 35.1°, and 36.1°, as determined by XRPD using a CuKα radiation source, for example, as shown in FIG. 7. In some embodiments, salt I-8b is in a crystalline solid form characterized by an X-ray powder diffraction pattern containing at least three characteristic peaks at diffraction angles (2θ±0.2°) selected from 12.7°, 13.5°, 15.8°, 16.1°, 17.2°, 17.9°, 19.8°, 20.1°, 20.8°, 23.8°, 24.6°, 26.9°, 29.3°, and 35.1° as determined by XRPD using a CuKα radiation source, for example, as shown in FIG. 7.

In some embodiments, the pharmaceutically acceptable salt is a salicylate salt of 2-(1H-indol-3-yl)-N,N-bis(methyl-d₃)ethan-1-amine-1,1,2,2-d₄ (I-8c) (i.e., a salicylate salt of compound I-8 depicted above). In some embodiments, salt I-8c is in a crystalline solid form characterized by an X-ray powder diffraction pattern containing at least three characteristic peaks at diffraction angles (2θ±0.2°) selected from 9.6°, 10.5°, 11.4°, 12.3°, 13.4°, 14.2°, 14.9°, 15.6°, 16.1°, 17.1°, 18.1°, 18.7°, 19.1°, 20.1°, 20.8°, 21.1°, 21.3°, 22.2°, 22.6°, 23.7°, 24.6°, 25.2°, 25.6°, 26.1°, 26.4°, 27.4°, 27.5°, 27.8°, 28.5°, 28.8°, 29.4°, 29.7°, 30.3°, 31.0°, 31.3°, 32.1°, 32.7°, 33.1°, 33.5° 34.4° and 35.0°, as determined by XRPD using a CuKα radiation source, for example, as shown in FIG. 8. In some embodiments, salt I-8c is in a crystalline solid form characterized by an X-ray powder diffraction pattern containing at least three characteristic peaks at diffraction angles (2θ±0.2°) selected from 9.6°, 10.5°, 14.9°, 17.1°, 18.1°, 19.1°, 20.1°, 20.8°, 21.1°, 21.3°, 24.6°, 25.6°, 28.5°, 28.8°, 29.4°, 30.3°, 31.3°, 32.1°, 33.5°, and 34.4°, as determined by XRPD using a CuKα radiation source, for example, as shown in FIG. 8. In some embodiments, salt I-8c is in a crystalline solid form characterized by an X-ray powder diffraction pattern containing at least three characteristic peaks at diffraction angles (2θ±0.2°) selected from 9.6°, 14.9°, 17.1°, 18.1°, 19.1°, 20.1°, 20.8°, 21.3°, 24.6°, 25.6°, 28.5°, and 32.1°, as determined by XRPD using a CuKα radiation source, for example, as shown in FIG. 8.

In some embodiments, the pharmaceutically acceptable salt of the present disclosure is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. In one non-limiting example, as a hydrate, the pharmaceutically acceptable salt herein may be a monohydrate, a dihydrate, etc.

Also disclosed herein is a method for stabilizing a compound of Formula (I). The method includes preparing a pharmaceutically acceptable salt of the compound of Formula (I).

Also disclosed herein is a method for preparing a pharmaceutically acceptable salt of the compound of Formula (I). Various methods and procedures for addition salt formation are known to those of ordinary skill in the art, any of which may be utilized in the present disclosure. In some embodiments, the method includes:
(a) suspending a free base of the compound of Formula (I) in a solvent or mixture of solvents;
(b) contacting an acid with the compound of Formula (I) to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, and octane, and combinations thereof. In some embodiments, the solvent is ethanol.

Suitable acids for use in the preparation of pharmaceutically acceptable acid addition salts may include those described heretofore. The acid may be an inorganic acid or an organic acid, with organic acids being preferred. In some embodiments, the acid is an organic acid selected from the group consisting of fumaric acid, benzoic acid, salicylic acid, succinic acid, oxalic acid, and glycolic acid. In some embodiments, the acid is an organic acid selected from the group consisting of fumaric acid, benzoic acid, salicylic acid, and succinic acid, with fumaric acid, benzoic acid, and salicylic acid being preferred.

In some embodiments, a stoichiometric (or superstoichiometric) quantity of the acid is contacted with the compound of Formula (I). In some embodiments, a sub-stoichiometric (e.g., 0.5 molar equivalents) quantity of the acid is contacted with the compound of Formula (I). The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

### Therapeutic Applications and Methods

Also disclosed herein is a method of treating a subject with a disease or disorder comprising administering to the subject a therapeutically effective amount of a pharmaceutically acceptable salt of a compound of Formula (I).

The dosage and frequency (single or multiple doses) of salts of the compounds administered can vary depending upon a variety of factors, including, but not limited to, the salt form/compound to be administered; the disease/condition being treated; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards or downwards.

Dosages may be varied depending upon the requirements of the subject and the compound or salt form thereof being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Routes of administration may include oral routes (e.g., enteral/gastric delivery, intraoral administration such buccal, lingual, and sublingual routes), parenteral routes (e.g., intravenous, intradermal, intraarterial, intraperitoneal, intrathecal, intraventricular, intraurethral, intrasternal, intracranial, intramuscular, intrasynovial, and subcutaneous administration), topical routes (e.g., conjuctival, intracorneal, intraocular, ophthalmic, auricular, transdermal, nasal (e.g., intranasal), vaginal, uretheral, respiratory, and rectal administration), inhalation, or others sufficient to affect a beneficial therapeutic response.

Administration may follow a continuous administration schedule, or an intermittent administration schedule. The administration schedule may be varied depending on the salt form and compound employed, the condition being treated, the administration route, etc. For example, administration may be performed once a day (QD), or in divided dosages throughout the day, such as 2-times a day (BID), 3-times a day (TID), 4-times a day (QID), or more. In some embodiments administration may be performed nightly (QHS). In some embodiments, the compounds/pharmaceutical compositions may be administered as needed (PRN). Administration may also be performed on a weekly basis, e.g., once a week, twice a week, three times a week, four times a week, every other week, every two weeks, etc., or less. The administration schedule may also designate a defined number of treatments per treatment course, for example, administration may be performed 1 time, 2 times, 3 times, 4 times, 5 times, 6 times, 7 times, or 8 times per treatment course. Other administration schedules may also be deemed appropriate using sound medical judgement.

The dosing can be continuous (7 days of administration in a week) or intermittent, for example, depending on the pharmacokinetics and a particular subject's clearance/accumulation of the drug. If intermittently, the schedule may be, for example, 4 days of administration and 3 days off (rest days) in a week or any other intermittent dosing schedule deemed appropriate using sound medical judgement. For example, intermittent dosing may involve administration of a single dose within a treatment course. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the compounds Formula (I)), and yet is entirely effective to treat the clinical symptoms demonstrated by the particular patient. This planning should involve the careful choice of active compound and salt form by considering factors such as compound potency, relative bioavailability, patient body weight, presence and severity of adverse side effects, preferred mode of administration, and the toxicity profile of the selected agent.

A therapeutically effective dose of the salt form of the compounds disclosed herein may vary depending on the variety of factors described above, but is typically that which provides the compound of Formula (I) in an amount of about 0.00001 mg to about 10 mg per kilogram body weight of the recipient per day, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg of the compound of Formula (I) (active).

The pharmaceutically acceptable salts of the compounds of the present disclosure may be administered at a psychedelic dose. Psychedelic dosing, by mouth or otherwise, may in some embodiments range from about 0.083 mg/kg, about 0.09 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, and up to about 5 mg/kg, about 4 mg/kg, about 3 mg/kg, about 2 mg/kg, about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg of the compound of Formula (I) (on an active basis). Higher dosing may also be used in some embodiments, as described above. In some embodiments, psychedelic doses are administered once, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose. These treatments are appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

Administration of sub-psychoactive (yet still potentially serotonergic concentrations) concentrations may be performed in some embodiments to achieve durable therapeutic benefits, with decreased toxicity, and may thus be suitable for microdosing. Sub-psychedelic dosing, by mouth or otherwise, may in some embodiments range from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and less than about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the compound of Formula (I) (on an active basis). Typically, sub-psychedelic doses are administered up to every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday.

Sub-psychedelic dosing can be carried out, for example, by transdermal delivery, subcutaneous administration, orally, etc., via modified, controlled, slow, or extended release dosage forms, including, but not limited to, depot dosage forms, implants, patches, and pumps, which can be optionally remotely controlled. Here, doses would achieve similar blood levels as low oral dosing, but would nevertheless be sub-psychedelic.

Sub-psychedelic doses can be used, e.g., for the chronic treatment or maintenance of a variety of diseases or disorders disclosed herein, examples of which include, but are not limited to, depression (e.g., MDD), inflammation, pain, and neuroinflammation.

The pharmaceutically acceptable salts of the compounds of present disclosure may be used for a maintenance regimen. As used herein, a “maintenance regimen” generally refers to the administration of pharmaceutically acceptable salts of the compounds of present disclosure following achievement of a target dose, e.g., following completion of an up-titration regimen, and/or following a positive clinical response, e.g., improvement of the patient's condition, either to the same drug or to a different drug. In some embodiments, the patient is administered a first drug for a therapeutic regimen and a second drug for a maintenance regimen, wherein the first and second drugs are different. For example, the patient may be administered a therapeutic regimen of a first drug which is not a dimethyltryptamine-type compound (e.g., the first drug is a serotonergic psychedelic such as LSD, psilocybin, MDMA, etc., or a non-psychedelic drug), followed by a salt form of a compound of the present disclosure (as the second drug) in a maintenance regimen. In another example, a different salt form of the present disclosure is used for the therapeutic regimen (first drug) than is used for the maintenance regimen (second drug). In some embodiments, the patient is administered the same salt form of the present disclosure for both a therapeutic regimen and a maintenance regimen. In any case, the maintenance dose may be used to ‘maintain’ the therapeutic response and/or to prevent occurrences of relapse. When the same salt form of the present disclosure is used for both the original therapeutic regimen and for the maintenance regimen, the maintenance dose may be at or below the therapeutic dose. In some embodiments, the maintenance dose is a psychedelic dose. In some embodiments, the maintenance dose is a sub-psychedelic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

In some embodiments, the pharmaceutically acceptable salt of the compound of Formula (I) is administered to the subject intravenously as a single bolus within a dosage range described above, e.g., about 0.1 mg/kg to about 0.8 mg/kg, or about 0.2 mg/kg to about 0.5 mg/kg, or about 0.3 mg/kg. In some embodiments, the pharmaceutically acceptable salt of the compound of Formula (I) is administered to the subject as a perfusion within the dosage range described above, e.g., about 0.1 mg/kg to about 0.8 mg/kg, or about 0.2 mg/kg to about 0.5 mg/kg, or about 0.45 mg/kg. The perfusion may be administered over a duration of about 5 minutes, about 10 minutes, about 20 minutes, about 30 minutes, about 40 minutes, about 50 minutes, about 1 hour, about 2 hours, about 3 hours, about 4 hours, about 5 hours, for example. The pharmaceutically acceptable salt of the compound of Formula (I) may be administered via perfusion at a rate of about 0.1 mg/min, 0.2 mg/min, 0.3 mg/min, 0.4 mg/min, 0.5 mg/min, 0.6 mg/min, 0.7 mg/min, 0.8 mg/min, 0.9 mg/min, 1 mg/min, 1.5 mg/min, 2 mg/min, 2.5 mg/min, 3 mg/min, 3.5 mg/min, 4 mg/min, 4.5 mg/min, 5 mg/min, or otherwise as deemed appropriate by a medical professional. In some embodiments, the pharmaceutically acceptable salt of the compound of Formula (I) is administered to the subject intravenously as a bolus within the dosage range described above, e.g., about 0.1 mg/kg to about 0.8 mg/kg, or about 0.2 mg/kg to about 0.5 mg/kg, or about 0.3 mg/kg, followed by a perfusion within the dosage range described above, e.g., about 0.1 mg/kg to about 0.8 mg/kg, or about 0.2 mg/kg to about 0.5 mg/kg, or about 0.45 mg/kg.

The subjects treated herein may have a disease or disorder associated with a serotonin 5-HT₂ receptor.

In some embodiments, the disease or disorder is a neuropsychiatric disease or disorder or an inflammatory disease or disorder.

In some embodiments, the disease or disorder is a central nervous system (CNS) disorder and/or psychological disorder, including, but not limited to, post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders (including, but not limited to, bipolar I disorder, bipolar II disorder, cyclothymic disorder), obsessive-compulsive disorder (OCD), generalized anxiety disorder (GAD), acute psychedelic crisis, social anxiety disorder, substance use disorders (including, but not limited to, alcohol use disorder, opioid use disorder, amphetamine use disorder, nicotine use disorder, smoking, and cocaine use disorder), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, eating disorders (including, but not limited to, anorexia nervosa, bulimia nervosa, binge-eating disorder, etc.), and paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido), and obesity.

In some embodiments, the methods provided herein are used to treat a subject with a depressive disorder. As used herein, the terms “depressive disorder” or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions. In some embodiments, the depressive disorder is major depressive disorder (MDD), atypical depression, bipolar disorder, catatonic depression, depressive disorder due to a medical condition, postpartum depression, premenstrual dysphoric disorder, seasonal affective disorder, or treatment-resistant depression (TRD).

In some embodiments, the disease or disorder is major depressive disorder (MDD). As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all- or almost all-activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e., depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g., vitamin B12 deficiency), autoimmune conditions (e.g., lupus and rheumatoid arthritis), viral or other infections (hepatitis, mononucleosis, herpes), back pain, and cancer (e.g., pancreatic cancer).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Mental Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with treatment-resistant depression (TRD). The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, 5 treatment attempts, or more. In some embodiments, the subject with treatment-resistant depression has been diagnosed with major depressive disorder and has failed to respond to 3 or more treatment attempts. In some embodiments, the subject with treatment resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the disease or disorder is an anxiety disorder. As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring. Examples of anxiety disorders include, but are not limited to, generalized anxiety disorder (GAD), social anxiety disorder, panic disorder, panic attack, a phobia-related disorder (e.g., phobias related to flying, heights, specific animals such as spiders/dogs/snakes, receiving injections, blood, etc., agoraphobia), separation anxiety disorder, selective mutism, anxiety due to a medical condition, post-traumatic stress disorder (PTSD), obsessive-compulsive disorder (OCD), substance-induced anxiety disorder, etc.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, the disease or disorder is generalized anxiety disorder (GAD). Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks. In some embodiments, the methods herein are provided to a subject with generalized anxiety disorder also having symptoms of depression. In some embodiments, after treating the symptom(s) is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is social anxiety disorder. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, body dysmorphic disorder, hoarding disorder, dermatillomania, trichotillomania, excoriation, substance-induced obsessive compulsive and related disorder, or an obsessive-compulsive disorder due to another medical condition, etc., or a combination thereof. In some embodiments, the disease or disorder is obsessive-compulsive disorder (OCD).

In some embodiments, at least one sign or symptom of an anxiety disorder is improved following treatment disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-I-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Dis-order 7-Item Scale, The Psychiatric Institute Trichotillomania Scale (PITS), The MGH Hairpulling Scale (MGH-HPS), The NEVIH Trichotillomania Severity Scale (NIMH-TSS), The NIMH Trichotillomania Impairment Scale (NEVIH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y—BOCS—II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y—BOCS-SR), The Obsessive-Compulsive Inventory-Re-vised (OCI-R), and the Dimensional Obsessive-Compulsive Scale (DOCS), or a combination thereof. In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is attention deficit disorder (ADD). ADD is most commonly diagnosed in children under the age of 16 who have 6 or more symptoms of inattention (5 or more for older teenagers) for at least 6 consecutive months, but no signs of hyperactivity/impulsivity. The symptoms of inattention include, but are not limited to, trouble paying attention, avoids long mental tasks such as homework, trouble staying on task, disorganized or forgetful, doesn't appear to listen when spoken to, doesn't pay close attention to details. Loses things often, makes careless mistakes, and struggles to follow through with instructions. In some embodiments, the disease or disorder is attention deficit hyperactivity disorder (ADHD). ADHD is marked by an ongoing pattern of inattention and/or hyperactivity-impulsivity. Hyperactivity-impulsivity symptoms may often include, but are not limited to, fidgeting or squirming while seated, leaving their seats in situations where staying seated is expected, running, dashing, or climbing around at inappropriate times, being unable to engage in hobbies quietly, being constantly in motion, talking excessively, answering questions before they are fully asked, having difficulty waiting for one's turn, and interrupting or intruding on others during conversations or activities.

In some embodiments, the disease or disorder is a headache disorder. As used herein, the term “headache disorder” refers to a disorder characterized by recurrent headaches. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination. Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve—the fifth cranial nerve whose primary function is to provide sensory and motor innervation to the face. Attacks are also associated with prominent unilateral cranial autonomic symptoms and subjects often experience agitation and restlessness during attacks. In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain. In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine.” Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves. In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, the frequency of headaches and/or related symptoms decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, at least one sign or symptom of headache disorder is improved following administration of a compound disclosed herein. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQoL-5D, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-5 Dimensions (EQ-5D), the Short-form 36 (SF-36), or a combination thereof. In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art. In some embodiments, the sign or symptom of the headache disorder is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, the disease or disorder is a substance use disorder. Substance addictions which can be treated using the methods herein include addictions to addictive substances/agents such as recreational drugs and addictive medications. Examples of addictive substances/agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine. In some embodiments, the disease or disorder is alcohol use disorder (AUD). In some embodiments, the disease or disorder is nicotine use (e.g., smoking) disorder, and the therapy is used for e.g., smoking cessation.

In some embodiments, the disclosure provides for the management of sexual dysfunction, which may include, but is not limited to, sexual desire disorders, for example, decreased libido; sexual arousal disorders, for example, those causing lack of desire, lack of arousal, pain during intercourse, and orgasm disorders such as anorgasmia; and erectile dysfunction; particularly sexual dysfunction disorders stemming from psychological factors.

In some embodiments, the disease or disorder is an eating disorder. As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring. In some embodiments, at least one sign or symptom of an eating disorder is improved following administration of a compound disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Asberg Depression Rating Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C-SSRS), the Life Changes Inventory (LCI), and combinations thereof. In some embodiments, treating according to the methods of the disclosure results in an improvement in an eating disorder compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is multiple sclerosis (MS). MS is a chronic, inflammatory disease of unknown etiology that involves an immune-mediated attack on the central nervous system. Myelin and the oligodendrocytes that form myelin appear to be the primary targets of the inflammatory attack, although the axons themselves are also damaged. MS disease activity can be monitored by cranial scans, including magnetic resonance imaging (MRI) of the brain, accumulation of disability, as well as rate and severity of relapses. The diagnosis of clinically definite MS as determined by the Poser criteria requires at least two neurological events suggesting demyelination in the CNS separated in time and in location. Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination. In some embodiments, the multiple sclerosis is relapsing multiple sclerosis. In some embodiments, the relapsing multiple sclerosis is relapsing-remitting multiple sclerosis. In some embodiments, the methods herein reduce a symptom of multiple sclerosis in the subject. In some embodiments, the symptom is a MRI-monitored multiple sclerosis disease activity, relapse rate, accumulation of physical disability, frequency of relapses, decreased tune to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work. In some embodiments, the methods herein decrease or inhibit reduction of brain volume. In some embodiments, brain volume is measured by percent brain volume change (PBVC). In some embodiments, the methods herein increase time to confirmed disease progression. In some embodiments, time to confirmed disease progression is increased by at least 10%, at least 20%, at least 30%, at least 40%, at least 50%, for example at least 20-60%. In some embodiments, the methods herein decrease abnormalities observed in whole Brain MTR histogram. In some embodiments, the accumulation of physical disability is measured by Kurtzke Expanded Disability Status Scale (EDSS) score. In some embodiments, the accumulation of physical disability is assessed by the time to confirmed disease progression as measured by Kurtzke Expanded Disability Status Scale (EDSS) score.

In some embodiments, the disease or disorder is a disease or disorder characterized by, or otherwise associated with, neuroinflammation. Treatment herein may provide cognitive benefits to subject's suffering from neurological and neurodegenerative diseases such as Alzheimer's disease and other dementia subtypes, Parkinson's disease, amyotrophc lateral sclerosis (ALS), and others where neuroinflammation is a hallmark of disease pathophysiology and progression. For example, emerging psychedelic research/clinical evidence indicates that psychedelics may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the methods of the present disclosure are used for the treatment of neurological and neurodegenerative disorders such as Alzheimer's disease, dementia subtypes, Parkinson's disease, and amyotrophc lateral sclerosis (ALS), where neuroinflammation is associated with disease pathogenesis. In some embodiments, the methods of the present disclosure are used for the treatment of Alzheimer's disease. In some embodiments, the methods of the present disclosure are used for the treatment of dementia. In some embodiments, the methods of the present disclosure are used for the treatment of Parkinson's disease. In some embodiments, the methods of the present disclosure are used for the treatment of amyotrophc lateral sclerosis (ALS). As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical compositions adapted for oral and/or extended-release dosing (e.g., subcutaneous) are appropriate for such treatment methods, with sub-psychedelic dosing being preferred. In some embodiments, treating according to the methods of the disclosure results in an improvement in cognition in subject's suffering from a neurological or neurodegenerative disease compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of a diary assessments, assessments by a clinical or caregiver, or clinical scales, described herein or known in the art.

Further, many of the behavioral issues associated with chronic and/or life-threatening illnesses, including neurodegenerative disorders such as Alzheimer's disease, may benefit from treatment with the compounds/salt forms disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Compounds/salt forms described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder, including neurological and neurodegenerative diseases. In some embodiments, the methods provided herein reduce at least one sign or symptom of a neurological and/or neurodegenerative disease. In some embodiments, the methods provided herein reduce at least one sign or symptom of a neurological and/or neurodegenerative disease (e.g., depression, anxiety, and/or stress) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment, e.g., according to any one of the diary assessments, assessments by a clinical or caregiver, or clinical scales, described herein or known in the art.

In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is amyotrophc lateral sclerosis (ALS). In some embodiments, the methods herein are used for the treatment of depression, anxiety, and/or stress associated with amyotrophc lateral sclerosis (ALS). In some embodiments, the disease or disorder is cancer related depression and anxiety. As discussed above, oral and/or extended-release dosing is appropriate for such applications, particularly when blood concentrations of active ingredient (e.g., a compound of Formula (I)) are kept below the psychedelic threshold.

In some embodiments, the methods disclosed herein are used for treatment of brain injury, including traumatic brain injury (TBI). TBI is an injury to the brain caused by an external force, and can be classified based on severity, ranging from mild traumatic brain injury (mTBI/concussion) to severe traumatic brain injury. TBI can also be categorized by mechanism, as either a closed or penetrating head injury, or other features such as whether it is occurring in a specific location or over a widespread area. TBI can result in physical, cognitive, social, emotional and behavioral symptoms, which may be treated herein. Some of the imaging techniques used for diagnosis and recovery markers include computed tomography (CT) and magnetic resonance imaging (MRIs).

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the compounds and compositions disclosed herein are used for treatment of tic disorders, including Tourette's Syndrome, which is also variously referred to as Tourette Syndrome, Tourette's Disorder, Gilles de la Tourette syndrome (GTS), or simply Tourette's or TS. The tic disorder may also be a pediatric autoimmune disorder associated with streptococcal infection (PANDAS), a transient tic disorder, a chronic tic disorder, or a tic disorder not otherwise specified (NOS). Tic disorders are defined in the Diagnostic and Statistical Manual of Mental Disorders (DSM) based on type (motor or phonic) and duration of tics (sudden, rapid, nonrhythmic movements), or similarly by the World Health Organization (ICD-10 codes). Tics are involuntary or semi-voluntary, sudden, brief, intermittent, repetitive movements (motor) or sounds (phonic) that are classified as simple or complex. Simple tics, for example, eye blinking or facial grimacing, are relatively easy to camouflage and may go largely unnoticed. Complex tics, such as body contortions, self-injurious behavior, obscene gestures, or shouting of socially inappropriate word or phrases, can appear to be purposeful actions and are particularly distressing. Transient tic disorders are generally characterized by multiple motor and/or phonic tics that occur for at least four weeks but less than 12 months. Chronic tic disorders are generally characterized by either single or multiple motor or phonic tics, but not both, which are present for more than a year. Tourette's Syndrome is diagnosed when both motor and phonic tics are present (although not necessarily concurrently) for more than one year. Thus, Tourette's syndrome (TS) is a chronic neuropsychiatric disorder characterized by the presence of fluctuating motor and phonic tics. The typical age of onset is between five and seven years. Affected children may become the target of teasing by peers, which in turn can result in low self-esteem, social isolation, poor school performance, depression and anxiety. In addition to causing social embarrassment, sudden, forceful tics can be painful, and violent head and neck tics have been reported to cause secondary neurologic deficits, such as compressive cervical myelopathy. Tourette's Syndrome patients are also at increased risk for obsessive-compulsive disorder (OCD), depression, and attention-deficit-hyperactivity disorder (ADHD). Tic disorder NOS is diagnosed when tics are present but do not meet the criteria for any specific tic disorder. The methods of the present disclosure can also be used for the treatment of tics induced as a side effect of a medication; tics associated with autism; and Tourettism (the presence of Tourette-like symptoms in the absence of Tourette's Syndrome (e.g., as a result of another disease or condition, such as a sporadic, genetic, or neurodegenerative disorder)).

In some embodiments, the disease or disorder may include conditions of the autonomic nervous system (ANS).

In some embodiments, the disease or disorder may include pulmonary disorders (e.g., asthma and chronic obstructive pulmonary disorder (COPD).

In some embodiments, the disease or disorder may include cardiovascular disorders (e.g., atherosclerosis).

In some embodiments, the disclosure provides for the management of different kinds of pain, including but not limited to cancer pain, e.g., refractory cancer pain; neuropathic pain; postoperative pain; opioid-induced hyperalgesia and opioid-related tolerance; neurologic pain; postoperative/post-surgical pain; complex regional pain syndrome (CRPS); shock; limb amputation; severe chemical or thermal burn injury; sprains, ligament tears, fractures, wounds and other tissue injuries; dental surgery, procedures and maladies; labor and delivery; during physical therapy; radiation poisoning; acquired immunodeficiency syndrome (AIDS); epidural (or peridural) fibrosis; orthopedic pain; back pain; failed back surgery and failed laminectomy; sciatica; painful sickle cell crisis; arthritis; autoimmune disease; intractable bladder pain; pain associated with certain viruses, e.g., shingles pain or herpes pain; acute nausea, e.g., pain that may be causing the nausea or the abdominal pain that frequently accompanies sever nausea; migraine, e.g., with aura; and other conditions including depression (e.g., acute depression or chronic depression), depression along with pain, alcohol dependence, acute agitation, refractory asthma, acute asthma (e.g., unrelated pain conditions can induce asthma), epilepsy, acute brain injury and stroke, Alzheimer's disease and other disorders. The pain may be persistent or chronic pain that lasts for weeks to years, in some cases even though the injury or illness that caused the pain has healed or gone away, and in some cases despite previous medication and/or treatment. In addition, the disclosure includes the treatment/management of any combination of these types of pain or conditions.

In some embodiments, the pain treated/managed is acute breakthrough pain or pain related to wind-up that can occur in a chronic pain condition. In some embodiments of the disclosure, the pain treated/managed is cancer pain, e.g., refractory cancer pain. In some embodiments of the disclosure, the pain treated/managed is post-surgical pain. In some embodiments of the disclosure, the pain treated/managed is orthopedic pain. In some embodiments of the disclosure, the pain treated/managed is back pain. In some embodiments of the disclosure, the pain treated/managed is neuropathic pain. In some embodiments of the disclosure, the pain treated/managed is dental pain. In some embodiments of the disclosure, the condition treated/managed is depression. In some embodiments of the disclosure, the pain treated/managed is chronic pain in opioid-tolerant patients.

In some embodiments, the disease or disorder is arthritis. Types of arthritis include osteoarthritis, rheumatoid arthritis, childhood arthritis, fibromyalgia, gout, and lupus. In some embodiments, the disease or disorder is osteoarthritis. In some embodiments, the disease or disorder is rheumatoid arthritis. In some embodiments, the disease or disorder is childhood arthritis. In some embodiments, the disease or disorder is gout. In some embodiments, the disease or disorder is lupus. In some embodiments, the disease or disorder is fibromyalgia. Fibromyalgia is a disorder characterized by widespread musculoskeletal pain accompanied by fatigue, sleep, memory and mood issues. Fibromyalgia is believed to amplify painful sensations by affecting brain and spinal cord processes involving painful and nonpainful signaling. Symptoms often begin after an event, such as physical trauma, surgery, infection or significant psychological stress. In other cases, symptoms gradually accumulate over time with no single triggering event. Women are more likely to develop fibromyalgia than are men. Many people who have fibromyalgia also have tension headaches, temporomandibular joint (TMJ) disorders, irritable bowel syndrome, anxiety and depression.

In some embodiments, the disease or disorder is inflammatory bowel disease (IBD). IBD is a term for two conditions, Crohn's disease and ulcerative colitis, that are characterized by chronic inflammation of the gastrointestinal (GI) tract, with such prolonged inflammation resulting in damage to the GI tract. Subjects suffering from IBD may experience persistent diarrhea, abdominal pain, rectal bleeding/bloody stools, weight loss, and fatigue. IBD may be diagnosed, and treatment may be monitored, using one or more of endoscopy, colonoscopy, contrast radiography, MRI, computed tomography (CT), stool samples, and blood tests, known by those of ordinary skill in the art.

In some embodiments, the disease or disorder is a sleep disorder such as narcolepsy, insomnia, nightmare disorder, sleep apnea, central sleep apnea, obstructive sleep apnea, hypopnea, sleep-related hypoventilation, restless legs syndrome, and jet lag. In some embodiments, the disease or disorder is narcolepsy.

In some embodiments, the disclosure relates to a method of treating a disease or condition by modulating N-methyl-D-aspartic acid (NMDA) activity, where the method comprises administering an effective amount of a pharmaceutically acceptable salt of a compound described herein (e.g., a compound of Formula (I)) to a subject in need thereof. In some embodiments, the disease or condition is selected from: levodopa-induced dyskinesia; dementia (e.g., Alzheimer's dementia), tinnitus, treatment resistant depression (TRD), major depressive disorder, melancholic depression, atypical depression, dysthymia, neuropathic pain, agitation resulting from or associated with Alzheimer's disease, pseudobulbar effect, autism, Bulbar function, generalized anxiety disorder, Alzheimer's disease, schizophrenia, diabetic neuropathy, acute pain, depression, bipolar depression, suicidality, neuropathic pain, or post-traumatic stress disorder (PTSD). In some embodiments, the disease or condition is a psychiatric or mental disorder (e.g., schizophrenia, mood disorder, substance induced psychosis, major depressive disorder (MDD), bipolar disorder, bipolar depression (BDep), post-traumatic stress disorder (PTSD), suicidal ideation, anxiety, obsessive compulsive disorder (OCD), and treatment-resistant depression (TRD)). In other embodiments, the disease or condition is a neurological disorder (e.g., Huntington's disease (HD), Alzheimer's disease (AD), or systemic lupus erythematosus (SLE)).

In some embodiments, the disclosure relates to a method of treating an ocular disease, such as uveitis, corneal disease, iritis, iridocyclitis, glaucoma, and cataracts, by administering ophthalmically a therapeutically effective amount of a pharmaceutically acceptable salt of a compound of Formula (I) to a subject in need thereof. For example, compounds/salt forms herein may be administered in the form of solutions, suspensions, ointments, emulsions, gel-forming solutions, powders for solutions, gels, ocular inserts, and implants. In some embodiments, the pharmaceutically acceptable salt of a compound of Formula (I) is administered in the form of an eye drop formulation.

The administering physician can provide a method of treatment that is prophylactic or therapeutic by adjusting the amount and timing of any of the salt forms of the compounds described herein on the basis of observations of one or more symptoms of the disorder or condition being treated.

In some embodiments, the subject is a mammal. In some embodiments, the mammal is a human.

Administration may be systemic or local. In some embodiments, administration to a mammal will result in systemic release of a compound of the present disclosure (for example, into the bloodstream). Methods of administration may include, but are not limited to, inhalation, for example via a nebulizer or inhaler; oral; gastric and rectal enteral routes; topical, such as transdermal and intradermal; and parenteral administration.

The compounds of the present disclosure possess advantageous metabolic degradation profiles which prevent high drug concentrations observed acutely after administration, while also enhancing brain levels of the active compound, so that in some embodiments the therapeutic doses may be reduced. Accordingly, the compound of Formula (I) (in salt form) may be administered at serotonergic, but sub-psychoactive concentrations to achieve durable therapeutic benefits, with decreased toxicity, e.g., toxicity associated with activation of 5-HT_{2B} receptors associated with valvular heart disease (Rothman, R. B., and Baumann, M. H., 2009, Serotonergic drugs and valvular heart disease, Expert Opin Drug Saf 8, 317-329). As a result, the salt forms of the compounds of Formula (I) may be suitable for microdosing.

In some embodiments, the pharmaceutically acceptable salt forms of the present disclosure may be used as a standalone therapy. In some embodiments, the pharmaceutically acceptable salt forms of the present disclosure may be used as an adjuvant/combination therapy. In some embodiments, the subject with a disorder is administered a pharmaceutically acceptable salt of a compound of the present disclosure and at least one additional therapy and/or therapeutic. In some embodiments, administration of an additional therapy and/or therapeutic is prior to administration of the pharmaceutically acceptable salt form of the present disclosure. In some embodiments, administration of an additional therapy and/or therapeutic is after administration of the pharmaceutically acceptable salt form of the present disclosure. In some embodiments, administration of an additional therapy and/or therapeutic is concurrent with administration of the pharmaceutically acceptable salt form of the present disclosure. In some embodiments, the additional therapy is an antidepressant, an anticonvulsant, lisdexamfetamine dimesylate, an antipsychotic, an anxiolytic, an anti-inflammatory drug, a benzodiazepine, an analgesic drug, a cardiovascular drug, an opioid antagonist, or combinations thereof.

In some embodiments, the additional therapy is a benzodiazepine. In some embodiments, the benzodiazepine is diazepam or alprazolam.

In some embodiments, the additional therapy is a N-methyl-D-aspartate (NMDA) receptor antagonist. In some embodiments, the NMDA receptor antagonist is ketamine. In some embodiments, the NMDA receptor antagonist is nitrous oxide.

In some embodiments, the additional therapy is an antidepressant. In some embodiments, an antidepressant indirectly affects a neurotransmitter receptor, e.g., via interactions affecting the reactivity of other molecules at a neurotransmitter receptor. In some embodiments, an antidepressant is an agonist. In some embodiments, an antidepressant is an antagonist. In some embodiments, an antidepressant acts (either directly or indirectly) at more than one type of neurotransmitter receptor. In some embodiments, an antidepressant is chosen from buproprion, citalopram, duloxetine, escitalopram, fluoxetine, fluvoxamine, milnacipran, mirtazapine, paroxetine, reboxetine, sertraline, and venlafaxine.

In some embodiments, the antidepressant is a tricyclic antidepressant (“TCA”), selective serotonin reuptake inhibitor (“SSRI”), serotonin and noradrenaline reuptake inhibitor (“SNRI”), dopamine reuptake inhibitor (“DRI”), noradrenaline reuptake Monoamine oxidase inhibitor (“MAOI”), including inhibitor (“NRU”), dopamine, serotonin and noradrenaline reuptake inhibitor (“DSNRI”), a reversible inhibitor of monoamine oxidase type A (RIMA), or combination thereof. In some embodiments, the antidepressant is a TCA. In some embodiments, the TCA is imipramine or clomipramine. In some embodiments, the antidepressant is an SRI. In some embodiments, the SSRI is escitalopram, paroxetine, sertraline, fluvoxamine, fluoxetine, or combinations thereof. In some embodiments, the SNRI is venlafaxine. In some embodiments, the additional therapy is pregabalin. In some embodiments, the pharmaceutically acceptable salt form of the present disclosure is administered in combination with a reversible inhibitor of monoamine oxidase type A (RIMA). Such a combination may be administered in the same dosage form or co-administered in separate dosage form. Such a combination may improve the bioavailability (e.g., oral bioavailability) of the compound of Formula (I) by minimizing enzymatic degradation mediated by MAO enzymes, such as deamination/oxidation processes. Examples of reversible inhibitors of monoamine oxidase type A (RIMAs) include, but are not limited to, moclobemide, tolaxatone, brofaromine, caroxazone, eprobemide, methylene blue, metralindole, minaprine, harmaline, harmine, rosiridin, amiflamine, cimoxatone, sercloremine, CX157, befloxatone, esuprone, tetrindole 5-(2-aminopropyl)indole (5-IT), a-methyl tryptamine (AMT), natural sources (e.g., syrian rue, tumeric, curcumin).

In some embodiments, the additional therapeutic is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapeutic is an anti-inflammatory drug. In some embodiments, the anti-inflammatory drug is a nonsteroidal anti-inflammatory drugs (NSAIDS), steroid, acetaminophen (COX-3 inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic agent, 3-hydroxy-3-methylglutaryl coenzyme A inhibitor (statins), estrogen, androgen, antiplatelet agent, antidepressant, Helicobacter pylori inhibitors, proton pump inhibitor, thiazolidinedione, dual-action compounds, or combination thereof.

In some embodiments, the additional therapeutic is an anti-anxiolytic. In some embodiments, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the additional therapy is an opioid antagonist. Non-limiting examples of opioid antagonists include naloxone, naltrexone, nalmefene, nalorphine, nalrphine dinicotinate, levallrphan, samidorphan, nalodeine, alvimopan, methylnaltrexone, naloxegol, 6-naltrexol, axelopran, bevenopran, methylsamidorphan, naldemedine, buprenorphine, decozine, butorphanol, levorphanol, nalbuphine, pentazocine, and phenazocine.

In some embodiments, the additional therapy is a cardiovascular drug. Non-limiting examples of cardiovascular drugs include digoxin or (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribohexopyranosyl) oxy]-12,14-dihydroxy-card-20(22)-enolide, lisinopril, captopril, ramipril, trandolapril, benazepril, cilazapril, enalapril, moexipril, perindopril, quinapril, fludrocortisone, enalaprilate, quinapril, perindopril, apixaban, dabigatran, edoxaban, heparin, rivaroxaban, warfarin, aspirin, clopidogrel, dipyridamole, prasugrel, ticagrelor, azilsartan, candesartan, eprosartan, irbesartan, losartan, olmesartan, telmisartan, valsartanscaubitril, acebutolol, atenolol, betaxolol, bisoprolol, metoprolol, nadolol, propranolol, sotalol, amlodipine, diltiazem, felodipine, nifedipine, nimodipine, nisolidipine, verapamil, statins, nicotinic acids, diuretics, vasodilators, and combinations thereof.

In some embodiments, the subject is administered at least one therapy. Non-limiting examples of therapies include transcranial magnetic stimulation, cognitive behavioral therapy, interpersonal psychotherapy, dialectical behavior therapy, mindfulness techniques, or acceptance, commitment therapy, or combinations thereof.

### Pharmaceutical Compositions

Also disclosed herein is a pharmaceutical composition comprising a pharmaceutically acceptable salt of a compound of Formula (I) and a pharmaceutically acceptable vehicle. The pharmaceutical compositions may contain one, or more than one, pharmaceutically acceptable salt of a compound of Formula (I).

“Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” herein refers to a diluent, adjuvant, excipient, carrier, or any other auxiliary or supporting ingredient with which a salt form of a compound of the present disclosure is formulated for administration to a mammal. Such pharmaceutical vehicles can be solids or liquids, such as water and oils, including those of petroleum, animal, vegetable or synthetic origin, such as peanut oil, soybean oil, mineral oil, sesame oil and the like. The pharmaceutical vehicles can be water, saline, juice (e.g., fruit juice), gum acacia, gelatin, starch paste, talc, keratin, colloidal silica, urea, and the like. The pharmaceutical vehicles can include one or more gases, e.g., to act as a carrier for administration via inhalation. In addition, auxiliary, stabilizing, thickening, lubricating, taste masking, coloring agents, and other pharmaceutical additives may be included in the disclosed compositions, for example those set forth hereinafter.

The pharmaceutical composition may comprise a single compound of Formula (I) in salt form, or a mixture of compounds of Formula (I), in either free base or salt form, i.e., the pharmaceutical composition may be formed from an isotopologue mixture of the disclosed compounds. In some embodiments, a subject compound of Formula (I) may be present in the pharmaceutical composition at a purity of at least 20% by weight, at least 30% by weight, at least 40% by weight, at least 50% by weight, at least 60% by weight, at least 70% by weight, at least 80% by weight, at least 90% by weight, at least 95% by weight, at least 99% by weight, based on a total weight of isotopologues of the compound of Formula (I) present in the pharmaceutical composition. For example, a pharmaceutical composition formulated with a DMT d-10 salt (salt form of 2-(1H-indol-3-yl)-N,N-bis(methyl-d₃)ethan-1-amine-1,1,2,2-d₄), as the subject compound, may additionally contain isotopologues of the subject compound, e.g., DMT d-9, a DMT d-8, etc., as free-base or salt forms, stereoisomers, solvates, or mixtures thereof. In some embodiments, the composition is substantially free of other isotopologues of the compound, in either free base or salt form, e.g., the composition has less than 20, 15, 10, 9, 8, 7, 6, 5, 4, 3, 2 or 1 or 0.5 mole percent of other isotopologues of the compound.

In some embodiments, any position in the compound having deuterium has a minimum deuterium incorporation that is greater than that found naturally occurring in hydrogen (about 0.016 atom %). In some embodiments, any position in the compound having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

In some embodiments, the pharmaceutical composition comprises a pharmaceutically acceptable vehicle and a pharmaceutically acceptable salt of at least two compounds of Formula (I) (a pharmaceutically acceptable salt of at least two compounds of Formula (I) being referred to as an “active salt mixture”). In some embodiments, the pharmaceutical composition comprises an active salt mixture comprising: (i) a pharmaceutically acceptable salt of DMT d-10, i.e., a pharmaceutically acceptable salt of 2-(1H-indol-3-yl)-N,N-bis(methyl-d₃)ethan-1-amine-1,1,2,2-d₄ (I-8), (ii) a pharmaceutically acceptable salt of DMT d-9, i.e., a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), and optionally (iii) a pharmaceutically acceptable salt of DMT d-8, i.e., a pharmaceutically acceptable salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12).

In some embodiments, the active salt mixture is a fumarate salt mixture, wherein the salt forms recited are fumarate salts. In some embodiments, the active salt mixture is a benzoate salt mixture, wherein the salt forms recited are benzoate salts. In some embodiments, the active salt mixture is a salicylate salt mixture, wherein the salt forms recited are salicylate salts. In some embodiments, the active salt mixture is a succinate salt mixture, wherein the salt forms recited are succinate salts.

In some embodiments, the pharmaceutical composition comprises an active salt mixture comprising: (i) from 60% to 98% by weight, from 65% to 97% by weight, from 70% to 96% by weight, from 75% to 95% by weight, from 80% to 94% by weight, from 85% to 93% by weight, from 90% to 92% by weight of a pharmaceutically acceptable salt of DMT d-10, i.e., a pharmaceutically acceptable salt of 2-(1H-indol-3-yl)-N,N-bis(methyl-d₃)ethan-1-amine-1,1,2,2-d₄ (I-8), or any range therebetween, based on a total weight of the active salt mixture, (ii) from 2% to 40% by weight, from 3% to 35% by weight, from 4% to 30% by weight, from 5% to 25% by weight, from 6% to 20% by weight, from 7% to 15% by weight, from 8% to 10% by weight, in sum, of a pharmaceutically acceptable salt of DMT d-9, i.e., the weight sum of a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), or any range therebetween, based on a total weight of the active salt mixture, and (iii) less than 10% by weight, less than 5% by weight, less than 3% by weight, less than 2% by weight, less than 1% by weight, less than 0.5% by weight, less than 0.25% by weight, or 0% by weight, in sum, of a pharmaceutically acceptable salt of DMT d-8, i.e., the weight sum of a pharmaceutically acceptable salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12), or any range therebetween, based on a total weight of the active salt mixture.

For example, in some embodiments, the pharmaceutical composition comprises an active salt mixture comprising: (i) from 90% to 98% by weight of a pharmaceutically acceptable salt of 2-(1H-indol-3-yl)-N,N-bis(methyl-d₃)ethan-1-amine-1,1,2,2-d₄ (I-8), or any range therebetween, based on a total weight of the active salt mixture, and (ii) from 2% to 10% by weight, in sum, of a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), or any range therebetween, based on a total weight of the active salt mixture. In some embodiments, the active salt mixture (and thus the pharmaceutical composition) contains no detectable amount of, or is otherwise substantially free of, a pharmaceutically acceptable salt of DMT d-8 (a pharmaceutically acceptable salt of one or more of 2-(1H-indol-3-yl)-N,N-bis(methyl-d₃)ethan-1-amine-1,1-d₂ (I-6), 2-(1H-indol-3-yl)-N,N-bis(methyl-d₃)ethan-1-amine-2,2-d₂ (I-7), and/or 2-(1H-indol-3-yl)-N,N-bis(methyl-d₃)ethan-1-amine-1,2-d₂ (I-12)), a pharmaceutically acceptable salt of DMT d-7, a pharmaceutically acceptable salt of DMT d-6 (a pharmaceutically acceptable salt of 2-(1H-indol-3-yl)-N,N-bis(methyl-d₃)ethan-1-amine (I-4)), a pharmaceutically acceptable salt of DMT d-5, a pharmaceutically acceptable salt of DMT d-4 (a salt of 2-(1H-indol-3-yl)-N,N-dimethylethan-1-amine-1,1,2,2-d₄ (I-5)), a pharmaceutically acceptable salt of DMT d-3, a pharmaceutically acceptable salt of DMT d-2 (a salt of one or more of 2-(1H-indol-3-yl)-N,N-dimethylethan-1-amine-1,1-d₂ (I-2) and/or 2-(1H-indol-3-yl)-N,N-dimethylethan-1-amine-2,2-d₂ (I-3)), a pharmaceutically acceptable salt of DMT d-1, and a pharmaceutically acceptable salt of DMT (a salt of 2-(1H-indol-3-yl)-N,N-dimethylethan-1-amine (I-1)). For example, in some embodiments, a weight, in sum, of pharmaceutically acceptable salts of isotopologues of compound of Formula (I) not listed in (i) or (ii), such as a pharmaceutically acceptable salt of DMT d-8, a pharmaceutically acceptable salt of DMT d-7, a pharmaceutically acceptable salt of DMT d-6, a pharmaceutically acceptable salt of DMT d-5, a pharmaceutically acceptable salt of DMT d-4, a pharmaceutically acceptable salt of DMT d-3, a pharmaceutically acceptable salt of DMT d-2, a pharmaceutically acceptable salt of DMT d-1, and a pharmaceutically acceptable salt of DMT, is less than 1% by weight, less than 0.75% by weight, less than 0.5% by weight, less than 0.4% by weight, less than 0.3% by weight, less than 0.25% by weight, less than 0.2% by weight, less than 0.1% by weight, or 0% by weight, based on a total weight of the active salt mixture.

In one example, the pharmaceutical composition comprises an active salt mixture comprising: (i) from 90% to 98% by weight of a fumarate salt of 2-(1H-indol-3-yl)-N,N-bis(methyl-d₃)ethan-1-amine-1,1,2,2-d₄ (I-8a), or any range therebetween, based on a total weight of the active salt mixture, and (ii) from 2% to 10% by weight, in sum, of a fumarate salt of one or more of 2-(1H-indol-3-yl)-N,N-bis(methyl-d₃)ethan-1-amine-1,2,2-d₃ (I-10a) and/or 2-(1H-indol-3-yl)-N,N-bis(methyl-d₃)ethan-1-amine-1,1,2-d₃ (I-11a), or any range therebetween, based on a total weight of the active salt mixture. In some embodiments, the active salt mixture (and thus the pharmaceutical composition) contains no detectable amount of, or is otherwise substantially free of, a fumarate salt of DMT d-8 (a fumarate salt of one or more of 2-(1H-indol-3-yl)-N,N-bis(methyl-d₃)ethan-1-amine-1,1-d₂ (I-6a), 2-(1H-indol-3-yl)-N,N-bis(methyl-d₃)ethan-1-amine-2,2-d₂ (I-7a), and/or 2-(1H-indol-3-yl)-N,N-bis(methyl-d₃)ethan-1-amine-1,2-d₂ (I-12a)), a fumarate salt of DMT d-7, a fumarate salt of DMT d-6 (a fumarate salt of 2-(1H-indol-3-yl)-N,N-bis(methyl-d₃)ethan-1-amine (I-4a)), a fumarate salt of DMT d-5, a fumarate salt of DMT d-4 (a fumarate salt of 2-(1H-indol-3-yl)-N,N-dimethylethan-1-amine-1,1,2,2-d₄ (I-5a)), a fumarate salt of DMT d-3, a fumarate salt of DMT d-2 (a fumarate salt of one or more of 2-(1H-indol-3-yl)-N,N-dimethylethan-1-amine-1,1-d₂ (I-2a) and/or 2-(1H-indol-3-yl)-N,N-dimethylethan-1-amine-2,2-d₂ (I-3a)), a fumarate salt of DMT d-1, and a fumarate salt of DMT (a fumarate salt of 2-(1H-indol-3-yl)-N,N-dimethylethan-1-amine (I-1a)). For example, in some embodiments, a weight, in sum, of fumarate salts of isotopologues of compound of Formula (I) not listed in (i) or (ii), such as a fumarate salt of DMT d-8, a fumarate salt of DMT d-7, a fumarate salt of DMT d-6, a fumarate salt of DMT d-5, a fumarate salt of DMT d-4, a fumarate salt of DMT d-3, a fumarate salt of DMT d-2, a fumarate salt of DMT d-1, and a fumarate salt of DMT, is less than 1% by weight, less than 0.75% by weight, less than 0.5% by weight, less than 0.4% by weight, less than 0.3% by weight, less than 0.25% by weight, less than 0.2% by weight, less than 0.1% by weight, or 0% by weight, based on a total weight of the active salt mixture.

In another example, the pharmaceutical composition comprises an active salt mixture comprising: (i) from 90% to 98% by weight of a benzoate salt of 2-(1H-indol-3-yl)-N,N-bis(methyl-d₃)ethan-1-amine-1,1,2,2-d₄ (I-8b), or any range therebetween, based on a total weight of the active salt mixture, and (ii) from 2% to 10% by weight, in sum, of a benzoate salt of one or more of 2-(1H-indol-3-yl)-N,N-bis(methyl-d₃)ethan-1-amine-1,2,2-d₃ (I-10b) and/or 2-(1H-indol-3-yl)-N,N-bis(methyl-d₃)ethan-1-amine-1,1,2-d₃ (I-11b), or any range therebetween, based on a total weight of the active salt mixture. In some embodiments, the active salt mixture (and thus the pharmaceutical composition) contains no detectable amount of, or is otherwise substantially free of, a benzoate salt of DMT d-8 (a benzoate salt of one or more of 2-(1H-indol-3-yl)-N,N-bis(methyl-d₃)ethan-1-amine-1,1-d₂ (I-6b), 2-(1H-indol-3-yl)-N,N-bis(methyl-d₃)ethan-1-amine-2,2-d₂ (I-7b), and/or 2-(1H-indol-3-yl)-N,N-bis(methyl-d₃)ethan-1-amine-1,2-d₂ (I-12b)), a benzoate salt of DMT d-7, a benzoate salt of DMT d-6 (a benzoate salt of 2-(1H-indol-3-yl)-N,N-bis(methyl-d₃)ethan-1-amine (I-4b)), a benzoate salt of DMT d-5, a benzoate salt of DMT d-4 (a benzoate salt of 2-(1H-indol-3-yl)-N,N-dimethylethan-1-amine-1,1,2,2-d₄ (I-5b)), a benzoate salt of DMT d-3, a benzoate salt of DMT d-2 (a benzoate salt of one or more of 2-(1H-indol-3-yl)-N,N-dimethylethan-1-amine-1,1-d₂ (I-2b) and/or 2-(1H-indol-3-yl)-N,N-dimethylethan-1-amine-2,2-d₂ (I-3b)), a benzoate salt of DMT d-1, and a benzoate salt of DMT (a benzoate salt of 2-(1H-indol-3-yl)-N,N-dimethylethan-1-amine (I-1b)). For example, in some embodiments, a weight, in sum, of benzoate salts of isotopologues of compound of Formula (I) not listed in (i) or (ii), such as a benzoate salt of DMT d-8, a benzoate salt of DMT d-7, a benzoate salt of DMT d-6, a benzoate salt of DMT d-5, a benzoate salt of DMT d-4, a benzoate salt of DMT d-3, a benzoate salt of DMT d-2, a benzoate salt of DMT d-1, and a benzoate salt of DMT, is less than 1% by weight, less than 0.75% by weight, less than 0.5% by weight, less than 0.4% by weight, less than 0.3% by weight, less than 0.25% by weight, less than 0.2% by weight, less than 0.1% by weight, or 0% by weight, based on a total weight of the active salt mixture.

In another example, the pharmaceutical composition comprises an active salt mixture comprising: (i) from 90% to 98% by weight of a salicylate salt of 2-(1H-indol-3-yl)-N,N-bis(methyl-d₃)ethan-1-amine-1,1,2,2-d₄ (I-8c), or any range therebetween, based on a total weight of the active salt mixture, and (ii) from 2% to 10% by weight, in sum, of a salicylate salt of one or more of 2-(1H-indol-3-yl)-N,N-bis(methyl-d₃)ethan-1-amine-1,2,2-d₃ (I-10c) and/or 2-(1H-indol-3-yl)-N,N-bis(methyl-d₃)ethan-1-amine-1,1,2-d₃ (I-11c), or any range therebetween, based on a total weight of the active salt mixture. In some embodiments, the active salt mixture (and thus the pharmaceutical composition) contains no detectable amount of, or is otherwise substantially free of, a salicylate salt of DMT d-8 (a salicylate salt of one or more of 2-(1H-indol-3-yl)-N,N-bis(methyl-d₃)ethan-1-amine-1,1-d₂ (I-6c), 2-(1H-indol-3-yl)-N,N-bis(methyl-d₃)ethan-1-amine-2,2-d₂ (I-7c), and/or 2-(1H-indol-3-yl)-N,N-bis(methyl-d₃)ethan-1-amine-1,2-d₂ (I-12c)), a salicylate salt of DMT d-7, a salicylate salt of DMT d-6 (a salicylate salt of 2-(1H-indol-3-yl)-N,N-bis(methyl-d₃)ethan-1-amine (I-4c)), a salicylate salt of DMT d-5, a salicylate salt of DMT d-4 (a salicylate salt of 2-(1H-indol-3-yl)-N,N-dimethylethan-1-amine-1,1,2,2-d₄ (I-5c)), a salicylate salt of DMT d-3, a salicylate salt of DMT d-2 (a salicylate salt of one or more of 2-(1H-indol-3-yl)-N,N-dimethylethan-1-amine-1,1-d₂ (I-2c) and/or 2-(1H-indol-3-yl)-N,N-dimethylethan-1-amine-2,2-d₂ (I-3c)), a salicylate salt of DMT d-1, and a salicylate salt of DMT (a salicylate salt of 2-(1H-indol-3-yl)-N,N-dimethylethan-1-amine (I-1c)). For example, in some embodiments, a weight, in sum, of salicylate salts of isotopologues of compound of Formula (I) not listed in (i) or (ii), such as a salicylate salt of DMT d-8, a salicylate salt of DMT d-7, a salicylate salt of DMT d-6, a salicylate salt of DMT d-5, a salicylate salt of DMT d-4, a salicylate salt of DMT d-3, a salicylate salt of DMT d-2, a salicylate salt of DMT d-1, and a salicylate salt of DMT, is less than 1% by weight, less than 0.75% by weight, less than 0.5% by weight, less than 0.4% by weight, less than 0.3% by weight, less than 0.25% by weight, less than 0.2% by weight, less than 0.1% by weight, or 0% by weight, based on a total weight of the active salt mixture.

In yet another example, the pharmaceutical composition comprises an active salt mixture comprising: (i) from 90% to 98% by weight of a succinate salt of 2-(1H-indol-3-yl)-N,N-bis(methyl-d₃)ethan-1-amine-1,1,2,2-d₄ (I-8d), or any range therebetween, based on a total weight of the active salt mixture, and (ii) from 2% to 10% by weight, in sum, of a succinate salt of one or more of 2-(1H-indol-3-yl)-N,N-bis(methyl-d₃)ethan-1-amine-1,2,2-d₃ (I-10d) and/or 2-(1H-indol-3-yl)-N,N-bis(methyl-d₃)ethan-1-amine-1,1,2-d₃ (I-11d), or any range therebetween, based on a total weight of the active salt mixture. In some embodiments, the active salt mixture (and thus the pharmaceutical composition) contains no detectable amount of, or is otherwise substantially free of, a succinate salt of DMT d-8 (a succinate salt of one or more of 2-(1H-indol-3-yl)-N,N-bis(methyl-d₃)ethan-1-amine-1,1-d₂ (I-6d), 2-(1H-indol-3-yl)-N,N-bis(methyl-d₃)ethan-1-amine-2,2-d₂ (I-7d), and/or 2-(1H-indol-3-yl)-N,N-bis(methyl-d₃)ethan-1-amine-1,2-d₂ (I-12d)), a succinate salt of DMT d-7, a succinate salt of DMT d-6 (a succinate salt of 2-(1H-indol-3-yl)-N,N-bis(methyl-d₃)ethan-1-amine (I-4d)), a succinate salt of DMT d-5, a succinate salt of DMT d-4 (a succinate salt of 2-(1H-indol-3-yl)-N,N-dimethylethan-1-amine-1,1,2,2-d₄ (I-5d)), a succinate salt of DMT d-3, a succinate salt of DMT d-2 (a succinate salt of one or more of 2-(1H-indol-3-yl)-N,N-dimethylethan-1-amine-1,1-d₂ (I-2d) and/or 2-(1H-indol-3-yl)-N,N-dimethylethan-1-amine-2,2-d₂ (I-3d)), a succinate salt of DMT d-1, and a succinate salt of DMT (a succinate salt of 2-(1H-indol-3-yl)-N,N-dimethylethan-1-amine (I-1d)). For example, in some embodiments, a weight, in sum, of succinate salts of isotopologues of compound of Formula (I) not listed in (i) or (ii), such as a succinate salt of DMT d-8, a succinate salt of DMT d-7, a succinate salt of DMT d-6, a succinate salt of DMT d-5, a succinate salt of DMT d-4, a succinate salt of DMT d-3, a succinate salt of DMT d-2, a succinate salt of DMT d-1, and a succinate salt of DMT, is less than 1% by weight, less than 0.75% by weight, less than 0.5% by weight, less than 0.4% by weight, less than 0.3% by weight, less than 0.25% by weight, less than 0.2% by weight, less than 0.1% by weight, or 0% by weight, based on a total weight of the active salt mixture.

In some embodiments, the pharmaceutically acceptable salt of the compound of Formula (I) is chemically pure, for example has a chemical purity of greater than 90%, 92%, 94%, 96%, 97%, 98%, or 99% by UPLC or HPLC. In some embodiments, the pharmaceutically acceptable salt of the compound of Formula (I) has no single impurity of greater than 1%, greater than 0.5%, greater than 0.4%, greater than 0.3%, or greater than 0.2%, measured by UPLC or HPLC. In some embodiments, the pharmaceutically acceptable salt of the compound of Formula (I) has a chemical purity of greater than 97 area %, greater than 98 area %, or greater than 99 area % by UPLC or HPLC. In some embodiments, the pharmaceutically acceptable salt of the compound of Formula (I) has no single impurity greater than 1 area %, greater than 0.5 area %, greater than 0.4 area %, greater than 0.3 area %, or greater than 0.2 area % as measured by UPLC or HPLC.

The pharmaceutical composition may be formulated with an enantiomerically pure compound of the present disclosure, e.g., a compound of Formula (I), or a racemic mixture of the compounds. As described herein, a racemic compound of Formula (I) may contain about 50% of the R- and S-stereoisomers based on a molar ratio (about 48 to about 52 mol %, or about a 1:1 ratio)) of one of the isomers. In some embodiments, a composition, medicament, or method of treatment may involve combining separately produced compounds of the R- and S-stereoisomers in an approximately equal molar ratio (e.g., about 48 to 52%). In some embodiments, a medicament or pharmaceutical composition may contain a mixture of separate compounds of the R- and S-stereoisomers in different ratios. In some embodiments, the pharmaceutical composition contains an excess (greater than 50%) of the R-enantiomer. Suitable molar ratios of R/S may be from about 1.5:1, 2:1, 3:1, 4:1, 5:1, 10:1, or higher. In some embodiments, a pharmaceutical composition may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In other embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included. Compositions may contain a mixture of the racemate and a separate compound of Formula (I), in salt form.

The pharmaceutical composition may be formulated with one or more crystalline forms of the pharmaceutically acceptable salt of the compound of Formula (I), including one or more crystalline polymorphs. In some embodiments, the pharmaceutical composition includes a mixture of crystalline polymorphs. In some embodiments, the pharmaceutical composition includes a single crystalline polymorph. The pharmaceutical composition may be formulated with one or more amorphous forms of the pharmaceutically acceptable salt of the compound of Formula (I), including one or more amorphic polymorphs. In some embodiments, the pharmaceutical composition includes a mixture of amorphous polymorphs. In some embodiments, the pharmaceutical composition includes a single amorphous polymorph. In some embodiments, the pharmaceutical composition includes a mixture of crystalline and amorphous polymorphs. In some embodiments, the pharmaceutical composition comprises a highly pure crystalline form of a pharmaceutically acceptable salt of a compound of Formula (I). For example, the pharmaceutical composition may comprise a pharmaceutically acceptable salt of a compound of Formula (I), wherein at least 90%, at least 95%, at least 99%, or at least 99.5% by weight of the pharmaceutically acceptable salt of the compound of Formula (I) present in the pharmaceutical composition is in crystalline form, e.g., as determined by X-ray powder diffraction and/or DSC. Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. Administration of the subject compounds may be systemic or local. In some instances, the pharmaceutical compositions are formulated for administration in accordance with routine procedures as a pharmaceutical composition adapted for oral, intravenous, subcutaneous, intramuscular, intradermal, transdermal, or inhalation administration, or other routes of administration as set forth herein, to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of vehicle will be determined in part by the particular compound, salt form, as well as by the particular method used to administer the composition. Accordingly, there is a wide variety of suitable formulations of the subject pharmaceutical compositions. Liquid form preparations include solutions and emulsions, for example, water, water/propylene glycol solutions, viscous aqueous solutions/suspensions, or organic solvents.

When administered to a mammal, the compounds and compositions of the present disclosure and pharmaceutically acceptable vehicles may be sterile. In some instances, an aqueous medium is employed as a vehicle e.g., when the subject compound is administered intravenously, subcutaneously, intramuscularly, intradermally, or via inhalation, such as water, saline solutions, viscous aqueous solutions/suspensions, and aqueous dextrose and glycerol solutions.

The quantity of the pharmaceutically acceptable salt of the compound of Formula (I) in a unit dose preparation may be varied or adjusted to provide (on active basis) e.g., from 0.001 mg to 1000 mg, 0.001 mg to 500 mg, 0.001 mg to 100 mg, or 0.001 mg to 75 mg, or 0.001 mg to 50 mg, or 0.001 mg to 25 mg, or 0.001 mg to 10 mg, or 0.01 mg to 8 mg, or 0.1 mg to 5 mg, or 1 mg to 3 mg, or 0.001 mg, 0.01 mg, 0.1 mg, 1 mg, 2 mg, 3 mg, 5 mg, 10 mg, 20 mg, about 30 mg, 40 mg, 50 mg, 75 mg, 100 mg, 150 mg, 200 mg, 300 mg, 400 mg, 500 mg, or any range therebetween, of the compound of Formula (I), or otherwise as deemed appropriate using sound medical judgment, according to the particular application, administration route, potency of the active component, etc. The composition can, if desired, also contain other compatible therapeutic agents.

In some embodiments, the pharmaceutical composition comprises at least 0.1% by weight, at least 0.5% by weight, at least 1% by weight, at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, and up to 99.9% by weight, up to 99.5% by weight, up to 99% by weight, up to 98% by weight, up to 97% by weight, up to 95% by weight, up to 90% by weight, up to 85% by weight, up to 80% by weight, up to 75% by weight, up to 70% by weight, up to 65% by weight, up to 60% by weight, up to 55% by weight of the pharmaceutically acceptable salt of the compound of Formula (I), based on a total weight of the pharmaceutical composition.

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens can, and in some cases should, be adjusted over time according to the individual need and the professional judgment of the person administering or supervising the administration of the formulations.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the compounds may be given continuously or temporarily suspended for a certain length of time (i.e., a “drug holiday”).

Once improvement of the patient's conditions has occurred, a maintenance dose may be administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

In some embodiments, the pharmaceutical composition has an onset of therapeutic action of 60, 50, 40, 30, 20, 10, 5 minutes or less. In some embodiments, the pharmaceutical composition has an acute effects duration of 480, 420, 360, 300, 240, 180, 120, 110, 100, 90, 80, 70, 60, 50, 40, 30, 20, 10, 5 minutes or less. In some embodiments, the pharmaceutical composition has a drug dissolution time of 120, 90, 60, 50, 40, 30, 20, 10, 5 seconds or less.

As described below, the pharmaceutical compositions of the present disclosure may be specially formulated for administration in solid, semi-solid, or liquid form, including those adapted for the following:
A. Oral administration, for example, drenches (aqueous or non-aqueous solutions or suspensions), tablets, films, or capsules, e.g., those targeted for buccal, sublingual, and systemic absorption, boluses, powders, granules, syrups, pastes for application to the tongue;
B. Parenteral administration, for example, by subcutaneous, intradermal, intramuscular, intravenous or epidural injection as, for example, a sterile solution or suspension, or a sustained release formulation, including viscous aqueous solutions/suspensions or others which generate a depot effect;
C. Topical application/transdermal administration, for example, as a cream, ointment, or a controlled release patch or spray applied to the skin, or application to orifices and/or mucosal surfaces such as intranasally, for example as an aqueous or non-aqueous solution, suspension, liposomal dispersion, emulsion, microemulsion or sol-gel, intravaginally or intrarectally, for example, as a pessary, cream or foam;
D. Modified release dosage forms, including delayed-, extended-, prolonged-, sustained-, pulsatile-, controlled-, accelerated-, fast-, targeted-, programmed-release, and gastric retention dosage forms, such modified release dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126); and
E. Inhalation administration, for example as an aerosol, preferably a mist.

Any of the pharmaceutical compositions described herein can comprise (as the active component) at least one of the pharmaceutically acceptable salts of a compound of Formula (I) as described herein. Tamper resistant dosage forms/packaging of any of the disclosed pharmaceutical compositions are contemplated.

### A. Oral Administration

The pharmaceutical compositions disclosed herein may be provided in solid, semisolid, or liquid dosage forms for oral administration, including both enteric/gastric delivery routes as well as intraoral routes such as buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. Oral dosage forms of the present disclosure may be optionally formulated with a monoamine oxidase (MAO) inhibitor, including a reversible inhibitor of monoamine oxidase type A (RIMA), to improve the oral bioavailability of the compound of Formula (I) by minimizing enzymatic degradation mediated by MAO enzymes, such as deamination/oxidation processes. In addition to the active ingredient(s), and any optional MAO inhibitor, the pharmaceutical compositions may contain one or more pharmaceutically acceptable vehicles, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present, e.g., from about 10%, about 20%, about 30%, about 40%, about 50%, about 60%, about 70%, about 80%, about 90%, about 99% by weight in the pharmaceutical compositions disclosed herein, or any range therebetween.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain e.g., from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL®200 (W. R. Grace Co., Baltimore, Md.) and CAB-O-SIL® (Cabot Co. of Boston, Mass.); and mixtures thereof. The pharmaceutical compositions disclosed herein may contain e.g., about 0.1 to about 5% by weight of a lubricant.

Suitable glidants include colloidal silicon dioxide, CAB-O-SIL® (Cabot Co. of Boston, Mass.), and asbestos-free talc.

Coloring agents include any of the approved, certified, water soluble FD&C dyes, and water insoluble FD&C dyes suspended on alumina hydrate, and color lakes and mixtures thereof.

A color lake is the combination by adsorption of a water-soluble dye to a hydrous oxide of a heavy metal, resulting in an insoluble form of the dye.

Flavoring agents include natural flavors extracted from plants, such as fruits, and synthetic blends of compounds which produce a pleasant taste sensation, such as peppermint and methyl salicylate.

Sweetening agents include sucrose, lactose, mannitol, syrups, glycerin, and artificial sweeteners, such as saccharin and aspartame.

Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

Suspending and dispersing agents include sodium carboxymethylcellulose, pectin, tragacanth, Veegum, acacia, hydroxypropyl methylcellulose, and polyvinylpyrolidone.

Preservatives include glycerin, methyl and propylparaben, benzoic add, sodium benzoate and alcohol.

Wetting agents include propylene glycol monostearate, sorbitan monooleate, diethylene glycol monolaurate, and polyoxyethylene lauryl ether.

Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many vehicles may serve several functions, even within the same formulation.

The pharmaceutical compositions disclosed herein may be formulated as compressed tablets, capsules, caplets, gelcaps and cap compositions, tablet triturates, chewable lozenges, rapidly dissolving tablets, multiple compressed tablets, or enteric-coated tablets, caplets, and caps, sugar-coated tablets, caplets, and caps, or film-coated tablets, caplets, and caps. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets. Oral pharmaceutical compositions of the disclosure may be in solid dosage forms intended for oral administration, e.g., obtained by dry granulation with single or multiple compressions of powders or granules. In some embodiments, the oral pharmaceutical compositions may be obtained by using wet granulation techniques. In some embodiments, the oral pharmaceutical compositions may be obtained by molding, heating/annealing, or extrusion techniques.

The tablet dosage forms may be prepared from the active ingredient in powdered, crystalline, or granular forms, alone or in combination with one or more vehicles described herein, including binders, disintegrants, controlled-release polymers, lubricants, diluents, and/or colorants. Flavoring and sweetening agents are especially useful in the formation of chewable tablets and lozenges.

In some embodiments, the pharmaceutical composition (e.g., a tablet composition formulated for oral administration such as a single-layer tablet composition), comprises any of the pharmaceutically acceptable salts of a compound of Formula (I) described herein, and a polymer.

In some embodiments, the tablet composition is a modified-release tablet adapted for sustained release and preferably maximum sustained release. In some embodiments, the release period of any of the compounds described herein (e.g., a compound of Formula (I)), in the formulations of the disclosure is greater than 4 hours, greater than 6 hours, greater than 8 hours, greater than 10 hours, greater than 12 hours, greater than 16 hours, greater than 20 hours, greater than 24 hours, greater than 28 hours, greater than 32 hours, greater than 36 hours, greater than 48 hours.

In some embodiments, the tablet composition is adapted for tamper resistance. In some embodiments, the tablet composition comprises polyethylene oxide (PEO), e.g., MW about 2,000 to about 7,000 KDa, in combination with HPMC. In some embodiments, the tablet composition may further comprise polyethylene glycol (PEG), e.g., PEG 8K. In some embodiments, the tablet composition may further comprise a polymer carrying one or more negatively charged groups, e.g., polyacrylic acid. In some embodiments, the tablet composition comprising PEO is further subjected to heating/annealing, e.g., extrusion conditions.

In some embodiments, the pharmaceutical composition comprises a combination of (i) a water-insoluble neutrally charged non-ionic matrix; (ii) a polymer carrying one or more negatively charged groups; and (iii) any of the pharmaceutically acceptable salts of a compound of Formula (I) described herein.

In some embodiments, the polymer carrying one or more negatively charged groups is selected from the group consisting of polyacrylic acid, polylactic acid, polyglycolic acid, polymethacrylate carboxylates, cation-exchange resins, clays, zeolites, hyaluronic acid, anionic gums, salts thereof, and mixtures thereof. In some embodiments, the anionic gum is selected from the group consisting of naturally occurring materials and semi-synthetic materials. In some embodiments, the naturally occurring material is selected from the group consisting of alginic acid, pectin, xanthan gum, carrageenan, locust bean gum, gum arabic, gum karaya, guar gum, and gum tragacanth. In some embodiments, the semi-synthetic material is selected from the group consisting of carboxymethyl-chitin and cellulose gum.

Moreover, without wishing to be bound by theory, in some embodiments, the role of the polymer carrying one or more negatively charged groups, e.g., moieties of acidic nature as in those of the acidic polymers described herein, surprisingly offers significant retention of any of the compounds described herein (e.g., a compound of Formula (I)), in the matrix. In some embodiments, this negative charge may be created in situ, for example, based on release of a proton due to pKa and under certain pH conditions or through electrostatic interaction/creation of negative charge. Further noting that acidic polymers may be the salts of the corresponding weak acids that will be the related protonated acids in the stomach; which, and without wishing to be bound by theory, will neutralize the charge and may reduce the interactions of any of the compounds described herein, with the matrix. In addition, the release matrix may be further complemented by other inactive pharmaceutical ingredients to aid in preparation of the appropriate solid dose form such as fillers, disintegrants, flow improving agents, lubricants, colorants, taste maskers.

In some embodiments, the water-insoluble neutrally charged non-ionic matrix is selected from cellulose-based polymers such as HPMC, alone or enhanced by mixing with components selected from the group consisting of starches; waxes; neutral gums; polymethacrylates; PVA; PVA/PVP blends; and mixtures thereof. In some embodiments, the cellulose-based polymer is hydroxypropyl methylcellulose (HPMC).

In some embodiments, the cellulose-based polymer is hydroxypropyl methylcellulose (HPMC). In some embodiments, the tablet composition comprises about 10 to 70%, 20 to 60%, or 30 to 50% hydroxypropyl methylcellulose by weight, about 10 to 30%, or about 15 to 20% starch by weight, or any combination thereof.

The dosage form may be an immediate release (IR) dosage form, examples of which include, but are not limited to, an immediate release (IR) tablet or an immediate release (IR) capsule. Dosage forms adapted for immediate release may include one or more pharmaceutically acceptable vehicles which readily disperse, dissolve, or otherwise breakdown in the gastric environment so as not to delay or prolong dissolution/absorption of the active ingredient(s). Examples of pharmaceutically acceptable vehicles for immediate release dosage forms include, but are not limited to, one or more binders/granulators, matrix materials, fillers, diluents, disintegrants, dispersing agents, solubilizing agents, lubricants, and/or performance modifiers. In some embodiments, the immediate release (IR) dosage form is an immediate release (IR) tablet comprising one or more of microcrystalline cellulose, sodium carboxymethylcellulose, magnesium stearate, mannitol, crospovidone, and sodium stearyl fumarate. In some embodiments, the immediate release (IR) dosage form comprises microcrystalline cellulose, sodium carboxymethylcellulose, and magnesium stearate. In some embodiments, the immediate release (IR) dosage form comprises mannitol, crospovidone, and sodium stearyl fumarate.

The pharmaceutical compositions disclosed herein may be formulated as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions disclosed herein may be in the form of orodispersible dosage forms (ODFs). Such dosage forms allow for pre-gastric absorption of the compounds herein, e.g., when administered intraorally through the mucosal linings of the oral cavity, e.g., buccal, lingual, and sublingual administration, for increased bioavailability and faster onset compared to oral administration through the gastrointestinal tract. Orodispersible dosage forms can be prepared by different techniques, such as freeze drying (lyophilization), molding, spray drying, mass extrusion or compressing. Preferably, the orodispersible dosage forms are prepared by lyophilization. In some embodiments, the orodispersible dosage forms disintegrate in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the orodispersible dosage forms dissolve in less than about 90 seconds, in less than about 60 seconds, or in less than about 30 seconds after being received in the oral cavity. In some embodiments, the orodispersible dosage forms disperse in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the pharmaceutical compositions are in the form of orodispersible dosage forms, such as orally disintegrating tablets (ODTs), having a disintegration time according to the United States Phamacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Phamacopeia (USP) disintegration test <701>, such as when adapted for extended release, for example 2 minutes, 3 minutes, 4 minutes, 5 minutes, 10 minutes, 15 minutes, 20 minutes, 25 minutes, 30 minutes, 45 minutes, 60 minutes, or any range therebetween, or longer, are also contemplated.

In some embodiments, the orodispersible dosage form is a sublingual dosage form to be disintegrated/dissolved under the tongue, whereby the contents (e.g., the compounds of the present disclosure) are absorbed through the mucous membrane beneath the tongue where they enter venous circulation. In some embodiments, the sublingual dosage form is disintegrated/dissolved under the tongue, whereby the contents are converted into a liquid or semi-solid dosage form, such as a solution, syrup, or paste upon mixing with the saliva, and subsequently swallowed. In some embodiments, the orodispersible dosage form is a buccal dosage form to be disintegrated/dissolved in the buccal cavity, whereby the contents (e.g., the compounds of the present disclosure) are absorbed through the oral mucosa lining the mouth where they enter venous circulation. In some embodiments, the buccal dosage form is disintegrated/dissolved in the buccal cavity, whereby the contents are converted into a liquid or semi-solid dosage form, such as a solution, syrup, or paste upon mixing with the saliva, and subsequently swallowed.

In some embodiments, the pharmaceutical compositions are in the form of orodispersible dosage forms such as fast dissolving tablets, also called orodispersible tablets or orally disintegrating tablets (ODTs) or fast dispersible tablets (FDTs). Fast dissolving tablets can be prepared by different techniques, such as freeze drying (lyophilization), molding, spray drying, mass extrusion or compressing. Preferably, the fast dissolving tablets are prepared by lyophilization. In some embodiments, the fast dissolving tablets disintegrate in less than about 90 seconds, in less than about 60 seconds, in less than about 30 seconds, in less than about 20, in less than about 10 seconds in the oral cavity, in less than about 5 seconds, or in less than about 2 seconds after being received in the oral cavity. In some embodiments, fast dissolving tablets dissolve in less than about 90 seconds, in less than about 60 seconds, or in less than about 30 seconds when placed in an aqueous environment such as the oral cavity.

In some embodiments, the pharmaceutical compositions are in the form of lyophilized FDTs. In some embodiments, the lyophilized FDTs are created by creating a porous matrix by subliming the water from a pre-frozen aqueous formulation of the drug containing matrix-forming agents and other vehicles such as those set forth herein, e.g., one or more lyoprotectants, preservatives, antioxidants, stabilizing agents, solubilizing agents, flavoring agents, etc. In some embodiments, the FDTs comprise two component frameworks of a lyophilized matrix system that work together to ensure the development of a successful formulation. In some embodiments, the first component is water-soluble polymers such as gelatin, dextran, alginate, and maltodextrin. This component maintains the shape and provides mechanical strength to the tablets (binder). In some embodiments, the second constituent is matrix-supporting/disintegration-enhancing agents such as sucrose, lactose, mannitol, xylitol, microcrystalline cellulose, calcium diphosphate, and/or starch, which acts by cementing the porous framework, provided by the water-soluble polymer and accelerates the disintegration of the FDT. In some embodiments, the lyophilized FDTs include gelatin and mannitol. In some embodiments, the lyophilized orodispersible dosage form (e.g., lyophilized FDT) includes gelatin, mannitol, and one or more of a lyoprotectant, a preservative, an antioxidant, a stabilizing agent, a solubilizing agent, a flavoring agent, etc., with particular mention being made to citric acid. A non-limiting example is Zydis® orally dispersible tablets (available from Catalent). In some embodiments, the formulation (e.g., Zydis® orally dispersible tablets) includes one or more water-soluble polymers, such as gelatin, one or more matrix materials, fillers, or diluents such as mannitol, a pharmaceutically acceptable salt of a compound of Formula (I), and optionally a lyoprotectant, a preservative, an antioxidant, a stabilizing agent, a solubilizing agent, and/or a flavoring agent. In some embodiments, the formulation (e.g., Zydis® orally dispersible tablets) includes gelatin, mannitol, a pharmaceutically acceptable salt of a compound of Formula (I), and an organic acid, non-limiting examples of which are citric acid and/or tartaric acid.

In some embodiments, the pharmaceutical compositions are in the form of orodispersible dosage forms such as lyophilized wafers. In some embodiments, the pharmaceutical compositions are in the form of lyophilized wafers protected for the long-term storage by a specialty packaging excluding moisture, oxygen and light. In some embodiments, the lyophilized wafers are created by creating a porous matrix by subliming the water from a pre-frozen aqueous formulation of the drug containing matrix-forming agents and other excipients such as lyoprotectants, preservatives, and flavoring agents. In some embodiments, the lyophilized wafer includes a thin water-soluble film matrix. In some embodiments, the wafers comprise two component frameworks of a lyophilized matrix system that work together to ensure the development of a successful formulation. In some embodiments, the first component is water-soluble polymers such as gelatin, dextran, alginate, and maltodextrin. This component maintains the shape and provides mechanical strength to the wafer (binder). In some embodiments, the second constituent is matrix-supporting/disintegration-enhancing agents such as sucrose, lactose, mannitol, xylitol, microcrystalline cellulose, calcium diphosphate, and/or starch, which acts by cementing the porous framework, provided by the water-soluble polymer and accelerates the disintegration of the wafer. In some embodiments, the lyophilized wafers include gelatin and mannitol. In some embodiments, the lyophilized wafers include gelatin, mannitol, and one or more of a lyoprotectant, a preservative, an antioxidant, a stabilizing agent, a solubilizing agent, a flavoring agent, etc., with particular mention being made to citric acid.

In some embodiments, the wafer can comprise a monolayer, bilayer, or trilayer. In some embodiments, the monolayer wafer contains an active agent and one or more vehicles, such as those set forth herein. In some embodiments, the bilayer wafer contains one or more vehicles, such as a solubilizing agent, in a first layer and an active agent in the second layer. This configuration allows the active agent to be stored separately from the vehicles and can increase the stability of the active agent and optionally increase the shelf life of the composition compared to the case where the vehicles and the active agent were contained in a single layer. For tri-layer wafers, each of the layers may be different or two of the layers, such as the upper and lower layers, may have substantially the same composition. In some embodiments, the lower and upper layers surround a core layer containing the active agent. In some embodiments, the lower and upper layers may contain one or more vehicles, such as a solubilizing agent. In some embodiments, the lower and upper layers have the same composition. Alternatively, the lower and upper layers may contain different vehicles or different amounts of the same vehicle. The core layer typically contains the active agent, optionally with one or more vehicles.

Pharmaceutically acceptable vehicles (e.g., carriers or excipients) which can be used in orodispersible dosage forms (ODFs) include, but are not limited to, a lyoprotectant, a preservative, an antioxidant, a stabilizing agent, a solubilizing agent, a flavoring agent, cyclodextrins, a bioadhesive agent, a permeation agent/absorption enhancer, or other pharmaceutically acceptable vehicles recited herein.

Examples of pharmaceutically acceptable lyoprotectants include, but are not limited to, disaccharides such as sucrose and trehalose, anionic polymers such as sulfobutylether-p-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include: (1) water soluble antioxidants, such as ascorbic acid, cysteine or salts thereof (cysteine hydrochloride), sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, glycerol, methionine, monothioglycerol, ascorbic acid, citric acid, polysorbate, arginine, cyclodextrins, microcrystalline cellulose, modified celluloses (e.g., carboxymethylcellulose, sodium salt), sorbitol, and cellulose gel.

Examples of pharmaceutically acceptable solubilizing agents (or dissolution aids) include, but are not limited to, citric acid, hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, fumaric acid, DL-malic acid, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, tartaric acid, dioctylsodium sulfosuccinate, zein, powdered skim milk, sorbitan trioleate, lactic acid, aluminum lactate, ascorbyl palmitate, hydroxyethylmethylcellulose, hydroxypropylmethylcelluloseacetate succinate, polyoxyethylene (105) polyoxypropylene (5) glycol, polyoxyethylene hydrogenated castor oil 60, polyoxyl 35 castor oil, poly(sodium 4-styrenesulfonate), polyvinylacetaldiethylamino acetate, polyvinyl alcohol, maleic acid, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate. Of these, in some embodiments, such as in ODT formulation, citric acid is preferred.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, lemon, lime, and lemon-lime.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Examples of suitable bioadhesive agents include, but are not limited to, cyclodextrin, cellulose derivatives such as hydroxypropyl methyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (polycarbophyl, Carbopol®, etc.); polyvinylpyrrolidone (PVP); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bioadhesive agents can also be used.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; menthol; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Disclosed herein are pharmaceutical compositions in modified release dosage forms, which comprise a compound as disclosed herein and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Further disclosed herein are pharmaceutical compositions in enteric coated dosage forms, which comprise a compound as disclosed herein and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise a compound as disclosed herein and one or more release controlling excipients or carriers for use in an effervescent dosage form. The pharmaceutical compositions may also comprise non-release controlling excipients or carriers.

Additionally, disclosed are pharmaceutical compositions in a dosage form that has an instant releasing component and at least one delayed releasing component, and is capable of giving a discontinuous release of the compound in the form of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise a compound as disclosed herein and one or more release controlling and non-release controlling excipients or carriers, such as those excipients or carriers suitable for a disruptable semipermeable membrane and as swellable substances.

Disclosed herein also are pharmaceutical compositions in a dosage form for oral administration to a subject, which comprise a salt form of a compound as disclosed herein and one or more pharmaceutically acceptable vehicles, enclosed in an intermediate reactive layer comprising a gastric juice-resistant polymeric layered material partially neutralized with alkali and having cation exchange capacity and a gastric juice-resistant outer layer.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

Further disclosed herein are pharmaceutical compositions in effervescent dosage form, which comprise a pharmaceutically acceptable salt of a compound of Formula (I), and one or more pharmaceutically acceptable vehicles, which may be release controlling vehicles and/or non-release controlling vehicles. Effervescent means that the dosage form, when mixed with liquid, including water, juice, saliva, etc., evolves a gas. In some embodiments, the effervescent dosage forms of the present disclosure comprise an organic acid and a source of carbon dioxide, referred to herein as an “effervescent couple.” Such effervescent dosage forms effervesce (evolve gas) through chemical reaction between the organic acid and the source of carbon dioxide, which takes place upon exposure to an aqueous environment, such as upon placement in water, juice, or other drinkable fluid, or from the aqueous environment in the oral cavity, such as saliva in the mouth. Specifically, the reaction between the organic acid and the source of carbon dioxide produces carbon dioxide gas upon contact with an aqueous medium such as water, juice, or saliva. While use of disintegrants are optional, effervescent dosage forms do not require a disintegrant as the evolution of the gas in situ facilitates the disintegration process.

For clarity, an “effervescent couple” refers to at least one organic acid and at least one source of carbon dioxide being contained in a dosage form, regardless of assembly—for example, the organic acid and the source of carbon dioxide can be admixed (as powders), layered on top of one another, agglomerated or otherwise “glued” together in granular form, or held separately from one another such as in separate layers within the dosage form. Further, the term “couple” in this context is not meant to be limited to only an organic acid and a source of carbon dioxide and is open to the inclusion of other materials unless specified otherwise; for example, effervescent agglomerates/granules made from bringing together (or “gluing”) an organic acid and a source of carbon dioxide may include other vehicles including binders (the “glue”) and the effervescent agglomerates/granules may nonetheless be referred to as an effervescent couple.

The organic acid may be a monoacid, a diacid, a triacid, a tetraacid, or may contain a higher number of acid groups. One organic acid or mixtures of organic acids may be used. In addition to an acid group(s) (e.g., one or more carboxylic acid moieties), the organic acid may also contain one or more hydroxyl functionalities as part of its structure (i.e., the organic acid may be a hydroxy acid). In some embodiments, the organic acid is an a-hydroxy acid. In some embodiments, the organic acid is a β-hydroxy acid. In some embodiments, the organic acid is a γ-hydroxy acid. Examples of hydroxy acids include, but are not limited to, glycolic acid, lactic acid, citric acid, tartaric acid, and malic acid. In some embodiments, the organic acid is citric acid and/or tartaric acid. In some embodiments, the organic acid is citric acid. In some embodiments, the organic acid is tartaric acid. In some embodiments, the organic acid is an enedioic acid, examples of which may include, but are not limited to, fumaric acid and maleic acid. In some embodiments, the organic acid is fumaric acid. In some embodiments, the organic acid is maleic acid. Mixtures and/or hydrates of the disclosed organic acid may also be used in the disclosed pharmaceutical compositions. In some embodiments, the organic acid is not a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the organic acid is not a benzoic acid (e.g., benzoic acid, 4-acetamidobenzoic acid, 2-acetoxybenzoic acid, salicylic acid, 4-amino-salicylic acid, gentisic acid, etc.).

The source of carbon dioxide may include, but is not limited to, sodium bicarbonate, sodium carbonate, potassium carbonate, potassium bicarbonate, magnesium carbonate, calcium carbonate, and sesquicarbonate. The source of carbon dioxide can be used singly, or in combination. In some embodiments, the source of carbon dioxide is sodium bicarbonate. In some embodiments, the source of carbon dioxide is sodium carbonate. In some embodiments, the source of carbon dioxide is potassium carbonate. In some embodiments, the source of carbon dioxide is potassium bicarbonate. However, reactants which evolve oxygen or other gases besides carbon dioxide, and which are safe for human consumption, are also contemplated for use in the disclosed effervescent dosage forms, in addition to or in lieu of the source of carbon dioxide. While not wishing to be bound by theory, it is believed that the effervescence can help quickly break up the dosage form, and in some routes of administration such as intraoral routes, can help reduce the perception of grittiness by providing a distracting sensory experience of effervescence.

In some embodiments, the effervescent dosage form is to be reconstituted in a drinkable fluid such as water or juice, thereby forming an oral liquid dosage form (e.g., solution), prior to consumption. In some embodiments, the effervescent dosage form is to be placed in the oral cavity, where contact with the aqueous environment (saliva) causes disintegration/dissolution of the dosage form along with effervescence. Here, the contents of the effervescent dosage form may be converted into a liquid or semi-solid dosage form, such as a solution, syrup, or paste upon mixing with the saliva, and subsequently swallowed. Alternatively, the effervescent dosage form may be an intraoral dosage form, e.g., a buccal, lingual, or sublingual dosage form, whereby placement in the aqueous environment (saliva) of the oral cavity causes disintegration/dissolution of the dosage form along with effervescence, and pre-gastric absorption of the contents through the oral mucosa. Such pre-gastric absorption may provide for increased bioavailability and faster onset compared to oral administration through the gastrointestinal tract. In some embodiments, the effervescent dosage form is a sublingual dosage form to be disintegrated/dissolved under the tongue, whereby the contents (e.g., the compounds of the present disclosure) are absorbed through the mucous membrane beneath the tongue where they enter venous circulation. In some embodiments, the effervescent dosage form is a buccal dosage form to be disintegrated/dissolved in the buccal cavity, whereby the contents (e.g., the compounds of the present disclosure) are absorbed through the oral mucosa lining the mouth where they enter venous circulation. Effervescent dosage forms may be advantageous for the treatment of pediatric/adolescent patients or patients that have general difficulty swallowing traditional dosage forms such as general tablets or capsules, since effervescent dosage forms can be reconstituted into easy to swallow liquid or semi-solid dosage forms or taken intraorally.

When adapted for intraoral administration, it may be beneficial to formulate the effervescent dosage form with a bioadhesive agent, in addition to the effervescent couple. “Bioadhesive agents” are substances which promote adhesion or adherence to a biological surface, such as mucous membranes. For example, bioadhesive agents are themselves capable of adhering to a biological surface when placed in contact with that surface (e.g., mucous membrane) in order to enable compositions of the disclosure to adhere to that surface, which promotes more efficient transfer of the contents from the dosage form to the biological surface. A variety of polymers known in the art can be used as bioadhesive agents, for example polymeric substances, preferably with an average (weight average) molecular weight above 5,000 g/mol. It is preferred that such polymeric materials are capable of rapid swelling when placed in contact with an aqueous medium such a water or saliva, and/or are substantially insoluble in water at room temperature and atmospheric pressure. Examples of suitable bioadhesive agents include, but are not limited to, cyclodextrin, cellulose derivatives such as hydroxypropyl methyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (polycarbophyl, Carbopol®, etc.); polyvinylpyrrolidone (PVP); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bioadhesive agents can also be used.

An effervescent couple can be coated with a pharmaceutically acceptable vehicle, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. Each component of the effervescent couple, e.g., the organic acid and/or the source of carbon dioxide, can also individually be coated with a pharmaceutically acceptable vehicle, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. The effervescent couple can also be mixed with previously lyophilized particles, such as one or more pharmaceutically active ingredients coated with a solvent protective or enteric coating.

The effervescent dosage form may be prepared by methods known to those skilled in the art, including, but not limited to, slugging, direct compression, roller compaction, dry or wet granulation, fusion granulation, melt-granulation, vacuum granulation, and fluid bed spray granulation, any of which may be optionally followed by compression/tableting.

The pharmaceutical compositions disclosed herein may be formulated as non-effervescent or effervescent granules and powders. The non-effervescent or effervescent granules and powders may be reconstituted into a liquid dosage form, or alternatively, compressed to form tablet dosage forms which are either non-effervescent or effervescent, respectively. Pharmaceutically acceptable vehicles used in the non-effervescent or effervescent granules or powders may include, but are not limited to, binders, granulators, fillers, diluents, sweetening agent, wetting agents, stabilizing agents, solubilizing agents, anti-caking agents, pH modifiers, or any other pharmaceutical vehicle described herein. In some embodiments, the pharmaceutically acceptable vehicle comprises an organic acid, such as glycolic acid, lactic acid, citric acid, tartaric acid, malic acid, fumaric acid, and/or maleic acid.

Pharmaceutically acceptable vehicles used in the effervescent granules or powders include an effervescent couple, i.e., an organic acid and a source of carbon dioxide. Effervescent powders may be produced by blending or admixing the organic acid and the source of carbon dioxide (the effervescent couple) and optionally any other desired pharmaceutically acceptable vehicle. Effervescent granules may be produced by physically adhering or “gluing” the effervescent couple (the organic acid and the source of carbon dioxide) together using an edible or pharmaceutically acceptable binder such as polyvinylpyrrolidone, polyvinyl alcohol, L-leucine, polyethylene glycol, gum arabic, or the like, including combinations thereof. These types of granules are made by processes generically known as “wet granulation.” Granulating solvents such as ethanol and/or isopropyl alcohol are often used to aid this type of granulation process. Since the effervescent couple is physically bound together in the granule, the gas generating reaction is usually quite vigorous, leading to rapid dissolution times. Another type of “wet granulation” product that is specific to effervescent products is known as “fusion” type granules. These granules are formed by reacting the organic acid and source of carbon dioxide with a small amount of water (or sometimes a hydrous alcohol granulating solvent, such as various commercial grades of ethanol or isopropyl alcohol) in a highly controlled way. Since the effervescent reaction generates carbon dioxide, fusion granules tend to be quite porous, which decreases their density and also their dissolution time. Accordingly, effervescent granules prepared by wet granulation or fusion type processes may be desirable for making orodispersible dosage forms or other dosage forms where quick dissolving/disintegrating properties are sought. Effervescent tablet dosage forms prepared through tableting, e.g., compression, of effervescent granules or powders are also included in the present disclosure.

The pharmaceutical compositions disclosed herein may be formulated as liquid and semisolid dosage forms, including emulsions, solutions, suspensions, elixirs, and syrups. In some embodiments, oral liquid dosage forms are prepared by reconstituting a solid dosage form disclosed herein (e.g., an effervescent dosage form) into a pharmaceutically acceptable liquid medium (e.g., aqueous medium) such as water, juice, or other drinkable fluid prior to use. In some embodiments, the oral liquid dosage form is prepared by reconstituting into a pharmaceutically acceptable aqueous medium a solid dosage form comprising a pharmaceutically acceptable salt of a compound of Formula (I), in crystalline form. In some embodiments, the oral liquid dosage form is prepared by reconstituting into a pharmaceutically acceptable aqueous medium a solid dosage form comprising a pharmaceutically acceptable salt of a compound of Formula (I), in amorphous form.

An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and optional preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, hydroxyethyl β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

The pharmaceutical compositions disclosed herein may be disclosed as non-effervescent or effervescent, granules and powders, to be reconstituted into a liquid dosage form. Pharmaceutically acceptable vehicles used in the non-effervescent granules or powders may include diluents, sweeteners, and wetting agents. Pharmaceutically acceptable vehicles used in the effervescent granules or powders may include organic acids and a source of carbon dioxide.

Coloring and flavoring agents can be used in all of the above dosage forms.

The pharmaceutical compositions disclosed herein may be co-formulated with other active ingredients which do not impair the desired therapeutic action, or with substances that supplement the desired action. One example is oral dosage forms formulated with a monoamine oxidase (MAO) inhibitor, including a reversible inhibitor of monoamine oxidase type A (RIMA), to improve the oral bioavailability of the compound of Formula (I) by minimizing enzymatic degradation mediated by MAO enzymes, such as deamination/oxidation processes.

### B. Parenteral Administration

The pharmaceutical compositions disclosed herein may be administered parenterally by injection, infusion, perfusion, or implantation, for local or systemic administration. Parenteral administration, as used herein, include intravenous, intradermal, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

In some embodiments, the pharmaceutical composition is in the form of an injectable (liquid) dosage form (e.g., for intravenous, intramuscular, subcutaneous, etc. administration). In some embodiments, injectable (liquid) dosage forms (e.g., for intravenous, intramuscular, subcutaneous, etc. administration) are prepared by reconstituting a solid dosage form disclosed herein into a pharmaceutically acceptable liquid medium such as water, saline solutions, viscous aqueous solutions/suspensions, water-miscible vehicles (e.g., organic solvents such as N-methyl-2-pyrrolidone), etc. prior to use. In some embodiments, the injectable (liquid) dosage form is prepared by reconstituting into a pharmaceutically acceptable liquid medium a solid dosage form comprising a pharmaceutically acceptable salt of a compound of Formula (I), in crystalline form. In some embodiments, the injectable (liquid) dosage form is prepared by reconstituting into a pharmaceutically acceptable liquid medium a solid dosage form comprising a pharmaceutically acceptable salt of a compound of Formula (I), in amorphous form.

The pharmaceutical compositions intended for parenteral administration may include one or more pharmaceutically acceptable vehicles, including, but not limited to, aqueous vehicles, water-miscible vehicles, non-aqueous vehicles, antimicrobial agents or preservatives against the growth of microorganisms, stabilizing agents, solubilizing agents, isotonic agents, buffering agents, antioxidants, local anesthetics, suspending and dispersing agents, wetting or emulsifying agents, complexing agents, sequestering or chelating agents, cryoprotectants, lyoprotectants, thickening or viscosity building agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate, acetate, and citrate buffers. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid.

Suitable complexing agents include, but are not limited to, cyclodextrins, including ca-cyclodextrin, β-cyclodextrin, hydroxypropyl-3-cyclodextrin, sulfobutylether-β-cyclodextrin, and sulfobutylether 7-O -cyclodextrin (CAPTISOL®, CyDex, Lenexa, Kans.). Suitable thickening or viscosity building agents include, for example, polyvinyl alcohol, polyvinyl pyrrolidone, methyl cellulose, hydroxypropyl methylcellulose, hydroxyethyl cellulose, carboxymethyl cellulose (e.g., sodium carboxymethyl cellulose), hydroxypropyl cellulose, chondroitin sulfate and salts thereof, hyaluronic acid and salts thereof, including crosslinked variations of any of the forgoing, and combinations of the foregoing.

In some embodiments, the pharmaceutical composition is in an injectable (liquid) dosage form. In some embodiments, the injectable (liquid) dosage form comprises a pharmaceutically acceptable salt of a compound of Formula (I), an aqueous vehicle (e.g., isotonic saline), a buffering agent (e.g., a citric acid buffer), optionally a pH adjusting agent (e.g., sodium hydroxide), and optionally an isotonic agent. In some embodiments, the injectable (liquid) dosage form comprises a pharmaceutically acceptable salt of a compound of Formula (I), an aqueous vehicle (e.g., isotonic saline), and a pH adjusting agent (e.g., sodium hydroxide), wherein the injectable (liquid) dosage form is formulated without a buffering agent (e.g., a citric acid buffer). In some embodiments, the injectable (liquid) dosage form is prepared by reconstituting a solid dosage form comprising a pharmaceutically acceptable salt of a compound of Formula (I) which is in crystalline form, into an aqueous vehicle such as isotonic saline. Reconstitution of the pharmaceutically acceptable salt of a compound of Formula (I) in crystalline form can be performed immediately prior to use.

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In some embodiments, the pharmaceutical compositions are disclosed as ready-to-use sterile solutions. In some embodiments, the pharmaceutical compositions are disclosed as sterile dry soluble products, including lyophilized powders and hypodermic tablets, to be reconstituted with a vehicle prior to use. In some embodiments, the pharmaceutical compositions are disclosed as ready-to-use sterile suspensions. In some embodiments, the pharmaceutical compositions are disclosed as sterile dry insoluble products to be reconstituted with a vehicle prior to use. In some embodiments, the pharmaceutical compositions are disclosed as ready-to-use sterile emulsions.

The pharmaceutical compositions may be formulated as a suspension, solid, semi-solid, or thixotropic liquid, for administration as an implanted depot or to generate a depot-like effect.

In some embodiments, the pharmaceutical compositions disclosed herein are dispersed in a solid inner matrix, which is surrounded by an outer polymeric membrane that is insoluble in body fluids but allows the active ingredient in the pharmaceutical compositions diffuse through. Suitable inner matrixes include, but are not limited to, polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate. Suitable outer polymeric membranes include, but are not limited to, polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

In some embodiments, the pharmaceutical composition is in the form of a viscous aqueous solution/suspension for injection to provide a slow/sustained absorption or depot-like effect. Here, pharmaceutical vehicles which build viscosity may be used, such as thickening or viscosity building agents including, but not limited to, polyvinyl alcohol, polyvinyl pyrrolidone, methyl cellulose, hydroxypropyl methylcellulose, hydroxyethyl cellulose, carboxymethyl cellulose (e.g., sodium carboxymethyl cellulose), hydroxypropyl cellulose, chondroitin sulfate and salts thereof, hyaluronic acid and salts thereof, and combinations of the foregoing. In some embodiments, the pharmaceutically acceptable vehicle comprises sodium carboxymethyl cellulose, hyaluronic acid and salts thereof, or a combination thereof. In some embodiments, the pharmaceutically acceptable vehicle comprises hyaluronic acid or a salt thereof. Such viscous aqueous solution/suspension dosage forms may be particularly well suited for subcutaneous or intramuscular administration, where the active ingredient can be slowly released from the injection site and absorbed over sustained periods, generating a depot-like release effect. Further, crosslinked versions of any of the forgoing may be utilized. The rate of release of the active ingredient can be controlled through the extent of cross-linking of any of the thickening or viscosity building agents described herein, or by controlling the rate that any of the forgoing are crosslinked through use, amount, or type of crosslinking agent employed. For example, a slow/sustained absorption or depot-like effect can be achieved through use or formation of a crosslinked hyaluronic acid at the injection site. In some embodiments, administration of a viscous aqueous solution/suspension dosage form, e.g., via subcutaneous or intramuscular injection, provides a release period of about 1 hour, about 2 hours, about 3 hours, about 4 hours, about 5 hours, about 6 hours, about 7 hours, about 8 hours, or any range therebetween, or longer.

In some embodiments, the pharmaceutical composition is formulated with a pharmaceutically acceptable salt of a compound of Formula (I) with poor aqueous solubility (e.g., a water solubility at 22° C. of less than 5 mg/mL, less than 4 mg/mL, less than 3 mg/mL, less than 2 mg/mL, less than 1 mg/mL, less than 0.5 mg/mL, less than 0.1 mg/mL), such as a fatty acid salt of a compound of Formula (I). Examples of fatty acid salt forms include, but are not limited to, those formed by contacting a compound of Formula (I) with adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, or caproic acid. Such pharmaceutical compositions may be particularly well suited for subcutaneous or intramuscular administration, where the active ingredient can slowly solubilize and be slowly released from the injection site and absorbed over sustained periods, generating a depot-like release effect. These “slow release” salts may be optionally formulated with thickening or viscosity building agents, e.g., in viscous aqueous solution/suspension formulations. In some embodiments, administration of a pharmaceutical composition formulated with a pharmaceutically acceptable salt of a compound of Formula (I) with poor aqueous solubility, e.g., via subcutaneous or intramuscular injection, provides a release period of about 1 hour, about 2 hours, about 3 hours, about 4 hours, about 5 hours, about 6 hours, about 7 hours, about 8 hours, or any range therebetween, or longer.

### C. Topical Administration

The pharmaceutical compositions disclosed herein may be administered topically to the skin, orifices, or mucosa. Topical administration, as described herein, includes, conjuctival, intracorneal, intraocular, ophthalmic, auricular, transdermal, nasal (e.g., intranasal), vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may contain the active ingredient(s) which may be mixed under sterile conditions with a pharmaceutically acceptable vehicle, and with any preservatives, buffers, absorption enhancers, propellants which may be required. Liposomes, micelles, microspheres, nanosystems, and mixtures thereof, may also be used. Dosage forms administered topically (e.g., intranasally) may be optionally formulated with a monoamine oxidase (MAO) inhibitor, including a reversible inhibitor of monoamine oxidase type A (RIMA), to improve the bioavailability of the compound of Formula (I) by minimizing enzymatic degradation mediated by MAO enzymes, such as deamination/oxidation processes.

Pharmaceutically acceptable vehicles suitable for use in the topical formulations disclosed herein include, but are not limited to, aqueous vehicles, water-miscible vehicles, non-aqueous vehicles, antimicrobial agents or preservatives against the growth of microorganisms, stabilizing agents, solubilizing agents, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening or viscosity building agents, and inert gases.

The ointments, pastes, creams and gels may contain, in addition to an active ingredient(s), vehicles, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient(s), vehicles such as lactose, talc, silicic acid, aluminum hydroxide, calcium silicates and polyamide powder, or mixtures of these substances. Sprays, such as those used for (intra)nasal administration, can additionally contain customary propellants, such as fluorohydrocarbons, chlorofluorohydrocarbons and volatile unsubstituted hydrocarbons, such as butane and propane.

Transdermal delivery devices (e.g., patches) may be used. Such dosage forms have the added advantage of providing controlled delivery of active ingredient(s) to the body. That is, the pharmaceutically acceptable salt of a compound of Formula (I) can be administered via a transdermal patch at a steady state concentration, whereby the active ingredient(s) is gradually administered over time, thus avoiding drug spiking and adverse events/toxicity associated therewith.

Transdermal patch dosage forms may be formulated with various amounts of the active agent (compound of Formula (I), provided in salt form), depending on the disease/condition being treated. The quantity of active agent in a unit dose preparation may be varied or adjusted e.g., 5 mg, 10 mg, 20 mg, about 30 mg, 40 mg, 50 mg, 75 mg, 100 mg, 150 mg, 200 mg, 300 mg, 400 mg, 500 mg, or from 5 mg to 25 mg, or 10 mg to 20 mg, or 12 mg to 18 mg, or 13 mg to 16 mg, or 14 mg to 15 mg, or otherwise as deemed appropriate using sound medical judgment, according to the particular application. Transdermal patches formulated with the disclosed compounds may be suitable for microdosing to achieve durable therapeutic benefits, with decreased toxicity. In some embodiments, compounds of the present disclosure may be administered via a transdermal patch at sub-psychoactive (yet still potentially serotonergic) concentrations, for example, over an extended period such as over a 8, 24, 48, 72, 84, 96, or 168 hour time period.

In addition to the active ingredient(s), and any optional pharmaceutically acceptable vehicles(s), the transdermal patch may also include one or more of a pressure sensitive adhesive layer, a backing, and a release liner, as is known to those of ordinary skill in the art.

Transdermal patch dosage forms can be made by dissolving or dispersing the active ingredient(s) in the proper medium. In some embodiments, the pharmaceutically acceptable salt of a compound of Formula (I) may be dissolved/dispersed directly into a polymer matrix forming the pressure sensitive adhesive layer. Such transdermal patches are called drug-in-adhesive (DIA) patches. Preferred DIA patch forms are those in which the active ingredient(s) is distributed uniformly throughout the pressure sensitive adhesive polymer matrix. In some embodiments, the active ingredient(s) may be provided in a layer containing the active ingredient(s) plus a polymer matrix which is separate from the pressure sensitive adhesive layer. In any case, the pharmaceutically acceptable salt of a compound of Formula (I) may optionally be formulated with suitable vehicles(s) such as carrier agents, permeation agents/absorption enhancers, humectants/crystallization inhibitors, etc. to increase the flux across the skin.

Examples of carrier agents may include, but are not limited to, C₈-C₂₂ fatty acids, such as oleic acid, undecanoic acid, valeric acid, heptanoic acid, pelargonic acid, capric acid, lauric acid, and eicosapentaenoic acid; C₈-C₂₂ fatty alcohols such as octanol, nonanol, oleyl alcohol, decyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; glycerol; ethyl acetate; acetoacetic ester; N-alkylpyrrolidone; cyclodextrins, such as α-cyclodextrin, p-cyclodextrin, γ-cyclodextrin, or derivatives such as 2-hydroxypropyl-β-cyclodextrin; and terpenes/terpenoids, such as limonene, linalool, myrcene, pinene such as a-pinene, caryophyllene, citral, eucolyptol, and the like; including mixtures thereof.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; menthol; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Examples of humectants/crystallization inhibitors include, but are not limited to, polyvinyl pyrrolidone-co-vinyl acetate, HPMC, polymethacrylate, and mixtures thereof.

The pressure sensitive adhesive layer may be formed from polymers including, but not limited to, acrylics (polyacrylates including alkyl acrylics), polyvinyl acetates, natural and synthetic rubbers (e.g., polyisobutylene), ethylenevinylacetate copolymers, polysiloxanes, polyurethanes, plasticized polyether block amide copolymers, plasticized styrene-butadiene rubber block copolymers, and mixtures thereof. The pressure-sensitive adhesive layer used in the transdermal patch of the present disclosure may be formed from an acrylic polymer pressure-sensitive adhesive, preferably an acrylic copolymer pressure sensitive adhesive. The acrylic copolymer pressure sensitive adhesive may be obtained by copolymerization of one or more alkyl (meth)acrylates (e.g., 2-ethylhexyl acrylate); aryl(meth)acrylates; arylalkyl (meth)acrylate; and (meth)acrylates with functional groups such as hydroxyalkyl (meth)acrylates (e.g., hydroxyethyl acrylate, 2-hydroxypropyl acrylate, 3-hydroxypropyl acrylate, 4-hydroxybutyl acrylate, 2-hydroxyethyl methacrylate, 2-hydroxypropyl methacrylate, 3-hydroxypropyl methacrylate, and 4-hydroxybutyl methacrylate), carboxylic acid containing (meth)acrylates (e.g., acrylic acid), and alkoxy (meth)acrylates (e.g., methoxyethyl acrylate); optionally with one or more copolymerizable monomers (e.g., vinylpyrrolidone, vinyl acetate, etc.). Specific examples of acrylic pressure-sensitive adhesives may include, but are not limited to, DURO-TAK products (Henkel) such as DURO-TAK 87-900A, DURO-TAK 87-9301, DURO-TAK 87-4098, DURO-TAK 87-2074, DURO-TAK 87-235A, DURO-TAK 87-2510, DURO-TAK 87-2287, DURO-TAK 87-4287, DURO-TAK 87-2516, DURO-TAK 387-2052, and DURO-TAK 87-2677.

The backing used in the transdermal patch of the present disclosure may include flexible backings such as films, nonwoven fabrics, Japanese papers, cotton fabrics, knitted fabrics, woven fabrics, and laminated composite bodies of a nonwoven fabric and a film. Such a backing is preferably composed of a soft material that can be in close contact with a skin and can follow skin movement and of a material that can suppress skin rash and other discomforts following prolonged use of the patch. Examples of the backing materials include, but are not limited to, polyethylene, polypropylene, polyethylene terephthalate, polybutylene terephthalate, polyethylene naphthalate, polystyrene, nylon, cotton, acetate rayon, rayon, a rayon/polyethylene terephthalate composite body, polyacrylonitrile, polyvinyl alcohol, acrylic polyurethane, ester polyurethane, ether polyurethane, a styrene-isoprene-styrene copolymer, a styrene-butadiene-styrene copolymer, a styrene-ethylene-propylene-styrene copolymer, styrene-butadiene rubber, an ethylene-vinyl acetate copolymer, or cellophane, for example. Preferred backings do not adsorb or release the active ingredient(s). In order to suppress the adsorption and release of the active ingredient(s), to improve transdermal absorbability of the active ingredient(s), and to suppress skin rash and other discomforts, the backing preferably includes one or more layers composed of the material above and has a water vapor permeability. Specific examples of backings may include, but are not limited to, 3M COTRAN products such as 3M COTRAN ethylene vinyl acetate membrane film 9702, 3M COTRAN ethylene vinyl acetate membrane film 9716, 3M COTRAN polyethylene membrane film 9720, 3M COTRAN ethylene vinyl acetate membrane film 9728, and the like.

The release liner used in the transdermal patch of the present disclosure may include, but is not limited to, a polyester film having one side or both sides treated with a release coating, a polyethylene laminated high-quality paper treated with a release coating, and a glassine paper treated with a release coating. The release coating may be a fluoropolymer, a silicone, a fluorosilicone, or any other release coating known to those of ordinary skill in the art. The release liner may have an uneven surface in order to easily take out the transdermal patch from a package. Examples of release liners may include, but are not limited to SCOTCHPAK products from 3M such as 3M SCOTCHPAK 9744, 3M SCOTCHPAK 9755, 3M SCOTCHPAK 9709, and 3M SCOTCHPAK 1022.

Other layers such as abuse deterrent layers formulated with one or more irritants (e.g., sodium lauryl sulfate, poloxamer, sorbitan monoesters, glyceryl monooleates, spices, etc.), may also be employed.

Methods disclosed herein using a transdermal patch dosage form provide for systemic delivery of small doses of active ingredient(s), preferably over extended periods of time such as up to 168 hour time periods, for example from 2 to 96 hours, or 4 to 72 hours, or 8 to 24 hours, or 10 to 18 hours, or 12 to 14 hours. In particular, the compound of Formula (I) can be delivered in small, steady, and consistent doses such that deleterious or undesirable side-effects can be avoided. In some embodiments, the compound of Formula (I) is administered transdermally at sub-psychoactive (yet still potentially serotonergic concentrations) concentrations.

An exemplary drug-in-adhesive (DIA) patch formulation may comprise 5 to 30 wt. % of a pharmaceutically acceptable salt of a compound of Formula (I), 30 to 70 wt. % pressure sensitive adhesive (e.g., DURO-TAK 387-2052, DURO-TAK 87-2677, and DURO-TAK 87-4098), 1 to 10 wt. % permeation agents/absorption enhancers (e.g., oleyloleate, oleyl alcohol, levulinic acid, diethylene glycol monoethyl ether, etc.), and 5 to 35 wt. % crystallization inhibitor (e.g., polyvinyl pyrrolidone-co-vinyl acetate, HPMC, polymethacrylate, etc.), each based on a total weight of the DIA patch formulation, though it should be understood that many variations are possible in light of the teachings herein.

Automatic injection devices offer a method for delivery of the compositions disclosed herein to patients. The compositions disclosed herein may be administered to a patient using automatic injection devices through a number of known devices, a non-limiting list of which includes transdermal, subcutaneous, and intramuscular delivery.

In some transdermal, subcutaneous, or intramuscular applications, a composition disclosed herein is absorbed through the skin. Passive transdermal patch devices often include an absorbent layer or membrane that is placed on the outer layer of the skin. The membrane typically contains a dose of a substance that is allowed to be absorbed through the skin to deliver the composition to the patient. Typically, only substances that are readily absorbed through the outer layer of the skin may be delivered with such transdermal patch devices.

Other automatic injection devices disclosed herein are configured to provide for increased skin permeability to improve delivery of the disclosed compositions. Non-limiting examples of structures used to increase permeability to improve transfer of a composition into the skin, across the skin, or intramuscularly include the use of one or more microneedles, which in some embodiments may be coated with a composition disclosed herein. Alternatively, hollow microneedles may be used to provide a fluid channel for delivery of the disclosed compositions below the outer layer of the skin. Other devices disclosed herein include transdermal delivery by iontophoresis, sonophoresis, reverse iontophoresis, or combinations thereof, and other technologies known in the art to increase skin permeability to facilitate drug delivery.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable vehicles utilized in rectal and vaginal suppositories include bases such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally. The terms “nasal,” “intranasal,” and the like refers to a route of administration, or dosage forms adapted for a route of administration, wherein the pharmaceutical dosage form is taken to, or through, the nose (e.g., nasal cavity). Similarly, a “nasal delivery device” or an “intranasal delivery device” is intended to mean an apparatus that administers an active ingredient into the nasal cavity. In some embodiments, the intranasal dosage form may be in the form of an aqueous or non-aqueous solution, suspension, liposomal dispersion, emulsion, microemulsion or sol-gel. Non-limiting examples of intranasal administration include introduction of a solution or suspension in the form of a nasal spray or drops (direct instillation) or intranasal application of a gel, emulsion or ointment. Relative to an oral dosage form such as a tablet or capsule, intranasal delivery provides for rapid absorption, faster onset of therapeutic action and avoidance of first pass metabolism. The amount of active ingredient absorbed depends on many factors. These factors include, but are not limited to, the drug concentration, the drug delivery vehicle, mucosal contact time, the venous drainage of the mucosal tissues, the degree that the drug is ionized at the pH of the absorption site, the size of the drug molecule, and its relative lipid solubility.

The pharmaceutical compositions of the present disclosure for nasal administration include a compound of the present disclosure, e.g., a pharmaceutically acceptable salt of a compound of Formula (I), and optionally a pharmaceutically acceptable vehicle including, but not limited to, permeation agents/absorption enhancers which promote nasal absorption of the active ingredient after nasal administration and agents to improve brain penetration of the drug following nasal administration, diluents, binders, lubricants, glidants, disintegrants, desensitizing agents, emulsifying agents, bioadhesive agents, solubilizing agents, suspending and dispersing agents, thickening or viscosity building agents, isotonic agents, pH adjusting agents, buffering agents, carriers, flavoring agents, sweetening agents, and mixtures thereof. In some embodiments, the active ingredient is present in the pharmaceutical composition in particulate form. In some embodiments, the particle size of the active ingredient is less than or equal to about 60 microns, which can help to ensure uniformity of any blends of the particles with other ingredients, or to provide an adequate dispersion in a liquid vehicle.

The transport of the active ingredient across normal mucosal surfaces (such as the nasal mucosa) can be enhanced by optionally combining it with a permeation agent/absorption enhancer. Examples of these permeation agents/absorption enhancers include, but are not limited to, cationic polymers, surface active agents, chelating agents, mucolytic agents, cyclodextrin, polymeric hydrogels, combinations thereof, and any other similar absorption promoting agents known to those of skill in the art. Representative examples of permeation agents/absorption enhancers include, but are not limited to, phospholipids, such as phosphatidylglycerol or phosphatidylcholine, lysophosphatidyl derivatives, such as lysophosphatidylethanolamine, lysophosphatidylcholine, lysophosphatidylglycerol, lysophosphatidylserine, or lysophosphatidic acid, polyols, such as glycerol or propylene glycol, fatty acid esters thereof such as glycerides, amino acids, and esters thereof, cyclodextrins, or others set forth herein. Gelling excipients or viscosity-increasing excipients can also be used.

The transport of the active ingredient across normal mucosal surfaces can also be enhanced by increasing the time in which the formulations adhere to the mucosal surfaces. Bioadhesive agents, for example, those which form hydrogels, exhibit muco-adhesion and controlled drug release properties and can be included in the intranasal compositions described herein. Representative bioadhesive agents capable of binding to the nasal mucosa include, but are not limited to, polycarbophil, polylysine, methylcellulose, sodium carboxymethylcellulose, hydroxypropylmethylcellulose, hydroxyethyl cellulose, pectin, Carbopol 934P, polyethylene oxide 600K, one or more poloxomers such as Pluronic F127 and/or Pluronic F-68, polyisobutylene (PIB), polyisoprene (PIP), polyvinyl pyrrolidone (PVP), polyvinyl alcohol (PVA), xanthan gum, guar gum, and locust bean gum. Other nasal delivery compositions are chitosan-based and are suitable to increase the residence time of the active ingredient on mucosal surfaces, which results in increasing its bioavailability. Thiolated polymeric vehicles that form covalent bonds with the cysteine-rich subdomains of the mucus membrane can also provide mucoadhesion, which prolongs the contact time between the active ingredient and the membrane.

The intranasal compositions can also include one or more preservatives. Representative preservatives include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as benzoic acid, sodium benzoate, potassium sorbate, parabens; or complex forming agents such as EDTA.

Intranasal dosage forms may also include ion-exchange resins, e.g., microspheres, which carry suitable anionic groups such as carboxylic acid residues, carboxymethyl groups, sulfopropyl groups and methylsulfonate groups. Ion-exchange resins, such as cation exchangers, can also be used. For example, pharmaceutical compositions may be formulated with chitosan, which is partially deacetylated chitin, or poly-N-acetyl-D-glucosamine, or a pharmaceutically acceptable salt thereof such as hydrochloride, lactate, glutamate, maleate, acetate, formate, propionate, malate, malonate, adipate, or succinate. Examples of non-ion-exchange resins (e.g., microspheres) which may be used include, but are not limited to starch, gelatin, collagen and albumin.

The pharmaceutical composition can also include an appropriate pH adjusting agent, including, but not limited to, sodium hydroxide, hydrochloric acid, citric acid, lactic acid, glutamic acid, maleic acid, acetic acid, formic acid, propionic acid, malic acid, malonic acid, adipic acid, and succinic acid.

Other ingredients such as diluents are cellulose, microcrystalline cellulose, hydroxypropyl cellulose, starch, hydroxypropyl methyl cellulose, dicalcium phosphate, calcium sulfate, lactose, sorbitol, sucrose, inositol, kaolin, mannitol, sodium chloride, and powdered sugar and the like.

Isotonic agents to adjust the tonicity of the composition may be added, including, but not limited to, sodium chloride, glucose, dextrose, mannitol, sorbitol, lactose, and the like.

Acidic, neutral, or basic buffering agents can also be added to the intranasal composition to control the pH, including, but not limited to, phosphate buffers, acetate buffers, and citrate buffers.

In addition to using permeation agents/absorption enhancers, which increase the transport of the active ingredient through the mucosa, and bioadhesive agents, which prolong the contact time of the active agent along the mucosa, the administration of the active ingredient can be controlled by using controlled release formulations. There are numerous particulate drug delivery vehicles known to those of skill in the art which can include the active ingredients and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active ingredient is in particulate form without added components, its release rate depends on the release of the active ingredient itself. Typically, the rate of absorption is enhanced by presenting the drug in a micronized form, wherein particles are below 20 microns in diameter. In contrast, if the active ingredient is in particulate form as a blend of the active agent and a polymer, the release of the active agent is controlled, at least in part, by the removal of the polymer, typically by dissolution, biodegradation, or diffusion from the polymer matrix. In some embodiments, the pharmaceutical composition is in the form of a viscous aqueous solution/suspension for intranasal administration to provide a slow/sustained release and absorption. Here, pharmaceutical vehicles which build viscosity may be used, such as thickening or viscosity building agents including, but not limited to, polyvinyl alcohol, polyvinyl pyrrolidone, methyl cellulose, hydroxypropyl methylcellulose, hydroxyethyl cellulose, carboxymethyl cellulose (e.g., sodium carboxymethyl cellulose), hydroxypropyl cellulose, chondroitin sulfate and salts thereof, hyaluronic acid and salts thereof, including crosslinked variants of any of the forgoing, and combinations of the foregoing. In some embodiments, the pharmaceutically acceptable vehicle comprises sodium carboxymethyl cellulose, hyaluronic acid and salts thereof, or a combination thereof. Such viscous aqueous solution/suspension dosage forms may be particularly well suited for intranasal dosage forms whereby the active ingredient is relatively short acting and/or where longer acting formulations are desirable, in that the active ingredient can be slowly released from the administration site and absorbed over sustained periods.

In some embodiments, the pharmaceutical composition is formulated with a pharmaceutically acceptable salt of a compound of Formula (I) with poor aqueous solubility (e.g., a water solubility at 22° C. of less than 5 mg/mL, less than 4 mg/mL, less than 3 mg/mL, less than 2 mg/mL, less than 1 mg/mL, less than 0.5 mg/mL, less than 0.1 mg/mL), such as a fatty acid salt of a compound of Formula (I). Examples of fatty acid salt forms include, but are not limited to, those formed by contacting a compound of Formula (I) with adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, or caproic acid. Such pharmaceutical compositions may be particularly well suited for intranasal dosage forms whereby the active ingredient is relatively short acting and/or where longer acting formulations are desirable, in that the active ingredient can be slowly released from the administration site and absorbed over sustained periods.

Other intranasal dosage forms and methods contemplated herein are disclosed in van Woensel M, et al. Formulations for Intranasal Delivery of Pharmacological Agents to Combat Brain Disease: A New Opportunity to Tackle GBM?Cancers (Basel). 2013 Aug. 14; 5(3):1020-48, incorporated herein by reference in its entirety.

Intranasal delivery devices are known in the art. Thus, any device suitable for delivery of drug to nasal mucosa may be used. Non-limiting examples of devices useful for the administration of liquid dosage forms include vapor devices (e.g., vapor inhalers), drop devices (e.g., catheters, single-dose droppers, multi-dose droppers, and unit-dose pipettes), mechanical spray pump devices (e.g., squeeze bottles, multi-dose metered-dose spray pumps, and single/duo-dose spray pumps), bi-directional spray pumps (e.g., breath-actuated nasal delivery devices), gas-driven spray systems/atomizers (e.g., single- or multi-dose HFA or nitrogen propellant-driven metered-dose inhalers, including traditional and circumferential velocity inhalers), and electrically powered nebulizers/atomizers (e.g., pulsation membrane nebulizers, vibrating mechanical nebulizers, and hand-held mechanical nebulizers). Non-limiting examples of devices useful for the administration of powder compositions (e.g., lyophilized or otherwise dried pooled compositions) include, but are not limited to, mechanical powder sprayers (e.g., handactuated capsule-based powder spray devices and handactuated powder spray devices, hand actuated gel delivery devices), breath-actuated inhalers (e.g., single- or multi-dose nasal inhalers and capsule-based single- or multi-dose nasal inhalers), and insufilators (e.g., breath-actuated nasal delivery devices).

Use of metered sprays for intranasal delivery can also be accomplished by including the active ingredient in a solution or dispersion in a suitable medium which can be administered as a spray. Representative devices of this type are disclosed in the following patents, patent applications, and publications: WO2003026559, WO2002011800, WO200051672, WO2002068029, WO2002068030, WO2002068031, WO2002068032, WO2003000310, WO2003020350, WO2003082393, WO2003084591, WO2003090812, WO200041755, and the pharmaceutical literature (See e.g., Bell, A. Intranasal Delivery Devices, in Drug Delivery Devices Fundamentals and Applications, Tyle P. (ed), Dekker, New York, 1988), Remington's Pharmaceutical Sciences, Mack Publishing Co., 1975, all of which are incorporated herein by reference.

In some embodiments, the pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable propellant, such as dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

The pharmaceutical compositions disclosed herein may be micronized to a size suitable for delivery, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as 1-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavoring agent, such as menthol and levomenthol, or sweeteners, such as saccharin or saccharin sodium.

In addition to the foregoing, the compounds of the present disclosure can also be administered intranasally in the form of irrigations and douches, as is known in the art. Nasal irrigation involves regularly flooding the nasal cavity with solution, which includes the drug. Nasal douches are typically used by filling a nasal douche with a solution including the drug, inserting the nozzle from the douche into one nostril, opening one's mouth to breathe, and causing the solution to flow into one nostril, rinse round the septum, and discharge from the other nostril.

The pharmaceutical compositions disclosed herein for topical administration may be formulated to be immediate release or modified release, including delayed-, sustained-, pulsed-, controlled-, targeted, and programmed release.

### D. Modified Release Dosage Forms

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. The pharmaceutical compositions in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

### 1. Matrix-Controlled Release Devices

The pharmaceutical compositions disclosed herein in a modified release dosage form may be fabricated using a matrix-controlled release device known to those skilled in the art (see, Takada et al in “Encyclopedia of Controlled Drug Delivery,” Vol. 2, Mathiowitz ed., Wiley, 1999).

In some embodiments, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents are water-swellable hydrophilic polymers, which are also referred to as “osmopolymers” and “hydrogels,” include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

The semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional vehicles as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical compositions disclosed herein are formulated as AMT controlled-release dosage form, which comprises an asymmetric osmotic membrane that coats a core comprising the active ingredient(s) and other pharmaceutically acceptable excipients or carriers. The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 m to about 3 mm, about 50 m to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other vehicles as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems.

### E. Inhalation Administration

The pharmaceutical compositions disclosed herein may be formulated for inhalation administration, e.g., for pulmonary absorption. Suitable preparations may include liquid form preparations such as those described above, e.g., solutions and emulsions, wherein the solvent or carrier is, for example, water, water/water-miscible vehicles such as water/propylene glycol solutions, or organic solvents, with optional buffering agents, which can be delivered as an aerosol, preferably a mist, with a carrier gas, such as air, oxygen, a mixture of helium and oxygen, or other gases and gas mixtures. The pharmaceutical compositions may also be formulated as a dry powder for insufflation, alone or in combination with an inert carrier such as lactose or phospholipids.

The pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable propellant, such as dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases.

Aqueous solutions suitable for inhalation use can be prepared by dissolving the pharmaceutically acceptable salt of a compound of Formula (I) in water. Suitable stabilizing agents and thickening agents can also be added. Emulsions suitable for inhalation use can be made by solubilizing the pharmaceutically acceptable salts of the compounds of Formula (I) in an aqueous medium and dispersing the solubilized form in a hydrophobic medium, optionally with viscous material, such as natural or synthetic gums, resins, methylcellulose, sodium carboxymethylcellulose, and other suspending agents.

Solutions or suspensions for use in a pressurized container, pump, spray, atomizer, or nebulizer may be formulated to contain a surfactant or other appropriate co-solvent, or a suitable alternative agent for dispersing, solubilizing, or extending release of the active ingredient disclosed herein, and optionally a propellant. Such surfactants or co-solvents may include, but are not limited to, Polysorbate 20, 60, and 80; Pluronic F-68, F-84, and P-103; cyclodextrin; polyoxyl 35 castor oil; sorbitan trioleate, oleic acid, or an oligolactic acid. Surfactants and co-solvents are typically employed at a level between about 0.01% and about 2% by weight of the pharmaceutical composition. Viscosity greater than that of simple aqueous solutions may be desirable in some cases to decrease variability in dispensing the formulations, to decrease physical separation of components of an emulsion of formulation, and/or otherwise to improve the formulation. Such viscosity building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose, chondroitin sulfate and salts thereof, hyaluronic acid and salts thereof, and combinations of the foregoing. Such agents, when desirable, are typically employed at a level between about 0.01% and about 2% by weight, between about 0.1% and about 1% by weight, between about 0.5% and about 0.8% by weight, of the pharmaceutical composition.

In the salt form, the compounds of Formula (I) can also be dissolved in organic solvents or aqueous mixtures of organic solvents. Organic solvents can be, for example, acetonitrile, chlorobenzene, chloroform, cyclohexane, 1,2-dichloromethane, dichloromethane, 1,2-dimethoxyethane, N,N-dimethylacetamide, N,N-dimethylformamide, 1,4-dioxane, 2-ethoxyethanol, ethylene glycol, formamide, hexane, methanol, ethanol, 2-methoxyethanol, methylbutylketone, methylcyclohexane, N-methyl-2-pyrrolidone, nitromethane, pyridine, sulfolane, tetralin, toluene, 1,1,2-trichloroethylene, or xylene, and like, including combinations thereof. Organic solvents can belong to functional group categories such as ester solvents, ketone solvents, alcohol solvents, amide solvents, ether solvents, hydrocarbon solvents, etc. each of which can be used.

Further description is provided below relating to pharmaceutical compositions and methods for inhalation administration.

### Inhalation Methods and Administration

Disclosed herein is a method of delivering a pharmaceutically acceptable salt of a compound of Formula (I) to a patient in need thereof comprising administering the pharmaceutically acceptable salt of the compound of Formula (I) dissolved in an aerosol, preferably a mist, via inhalation. Delivery of the pharmaceutically acceptable salt of a compound of Formula (I) may be useful in the treatment of a disease or disorder, such as a disease or disorder associated with a serotonin 5-HT₂ receptor, e.g., inter alia, a central nervous system (CNS) disorder and/or psychological disorder, as described herein. Preferably, the aerosol is generated without externally added heat (this does not exclude minor temperature increases caused by the formation of the aerosol itself, such as with a vibrating mesh or other nebulizer. However, such minor temperature increases can often be offset by vaporization of the drug, which results in cooling of the composition). The pharmaceutically acceptable salt of the compound of Formula (I) can be any set forth herein. In some embodiments, the pharmaceutically acceptable salt of the compound of Formula (I) can be delivered as an aerosol, preferably a mist, with a carrier, such as air, oxygen, or a mixture of helium and oxygen, or other gas mixtures including therapeutic gas mixtures. The carrier can in some instances be a mixture of helium and oxygen heated to about 50° C. to about 60° C.

Additionally, by administration via inhalation, the pharmaceutically acceptable salt of the compound of Formula (I) can be delivered systemically to the patient's central nervous system. The carrier gas, e.g., air, oxygen, a mixture of helium and oxygen, or other gases and gas mixtures, can be heated to about 50° C. to about 60° C., or to about 55° C. to about 56° C. When a mixture of helium and oxygen is used as the carrier, the helium can be present in the mixture of oxygen and helium at about 50%, 60%, 70%, 80% or 90% by volume, and the oxygen can be present in the mixture at about 50%, 40%, 30%, or 10% by volume, or any range therebetween.

The method can further comprise administering a pretreatment inhalation therapy prior to administration of the aerosol comprising the pharmaceutically acceptable salt of the compound of Formula (I). The pretreatment can comprise administering via inhalation of a mixture of helium and oxygen heated to about 90° C., to about 92° C., to about 94° C., to about 96° C., to about 98° C., to about 100° C., to about 105° C., to about 110° C., to about 115° C., to about 120° C., or any range therebetween, to the patient.

The method can comprise (i) administering via inhalation a mixture of helium and oxygen heated to about 90° C. to about 120° C. to the patient, followed by (ii) administering via inhalation a mixture of helium and oxygen heated to about 50° C. to about 60° C. and the aerosol comprising the pharmaceutically acceptable salt of the compound of Formula (I) to the patient and then repeating steps (i) and (ii). Steps (i) and (ii) can be repeated 1, 2, 3, 4, 5, or more times.

In some embodiments, the present disclosure provides a method of treating a central nervous system (CNS) disorder and/or psychological disorder comprising administering, via inhalation, a pharmaceutically acceptable salt of the compound of Formula (I) in the form of an aerosol, preferably a mist. The pharmaceutically acceptable salt of the compound of Formula (I) can be delivered as an aerosol along with a carrier gas e.g., air, oxygen, a mixture of helium and oxygen, or other gases and gas mixtures. The mixture of helium and oxygen can be heated to about 50° C. to about 60° C. prior to administering the aerosol comprising the pharmaceutically acceptable salt of the compound of Formula (I) to the patient.

The central nervous system and/or psychological disorder can be, for example, any of those disclosed herein, with specific mention being made to a substance use disorder (e.g., alcohol use disorder), generalized anxiety disorder (GAD), GAD with depression, social anxiety disorder, and treatment-resistant depression (TRD).

In some embodiments, the pharmaceutically acceptable salt of the compound of Formula (I) is delivered by inhalation to the patient's central nervous system resulting in an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

The pharmaceutically acceptable salt of the compound of Formula (I) can, in some embodiments, be administered via aerosol inhalation at doses of about 1 g to about 200 mg or more (or any range between about 1 g to about 200 mg), e.g., about 1 μg, 2 μg, 5 μg, 6 μg, 10 ag, 13 μg, 15 μg, 20 μg, 30 μg, 40 μg, 50 μg, 60 μg, 70 ag, 80 μg, 90 ag, 100 ag, 110 ag, 120 ag, 130 g, 140 μg, 150 μg, 160 μg, 170 μg, 180 μg, 190 ag, 200 ag, 210 ag, 220 μg, 230 ag, 240 ag, 250 g, 260 μg, 270 μg, 280 ag, 290 μg, 300 ag, 400 μg, 500 ag, 1.0 mg, 2.0 mg, 3.0 mg, 4.0 mg, 5.0 mg, 6.0 mg, 7.0 mg, 8.0 mg, 9.0 mg, 10.0 mg, 20.0 mg, 30.0 mg, 40.0 mg, 50.0 mg, 60.0 mg, 70.0 mg, 80.0 mg, 90.0 mg, 100.0 mg, 150.0 mg, 200.0 mg, or more, per inhalation session. In some embodiments, a subject can have 1, 2, 3, 4, 5 or more inhalation sessions a day. In some embodiments, a subject can have 1, 2, 3, 4, 5 or more inhalation sessions every other day, once a week, twice a week, or three times a week. In some embodiments, a subject can have 1, 2, 3, 4, 5 or more inhalation sessions every other month, twice a month, three times a month, or four times a month. In some embodiments, a subject can have 1, 2, 3, 4, 5, 6, 7, 8, or more inhalation sessions per treatment course, such as within a 28-day time period.

### Aerosols

In some embodiments, methods of delivering the disclosed salt forms by aerosol inhalation are provided. An aerosol, preferably a mist, can be delivered using, air, oxygen, a mixture of helium and oxygen, or other gases and gas mixtures, as a carrier gas. The carrier gas can be delivered at room temperature or heated. In some embodiments, an aerosol, preferably a mist comprising a pharmaceutically acceptable salt of the compound of Formula (I) is delivered via inhalation using heated helium-oxygen (HELIOX) mixtures. Due to very low viscosity of helium the helium-oxygen mixtures generate gaseous streams characterized by laminar flow that is a highly desirable feature for reaching out into the deep lung areas and reducing deposition of the drug in the respiratory tract, one of the major obstacles in dose delivery via inhalation. A patient can inhale a dissolved salt form disclosed herein as a mist into an alveolar region of the patient's lungs. The compound of Formula (I) can then be delivered to a fluid lining of the alveolar region of the lungs and can be systemically absorbed into patient blood circulation. Advantageously, these formulations can be effectively delivered to the blood stream upon inhalation to the alveolar regions of the lungs.

Devices suitable for delivery of heated or unheated carrier gas (e.g., air, oxygen, or helium-oxygen mixtures) include, for example, continuous mode nebulizers Flo-Mist (Phillips) and Hope (B&B Medical Technologies) and the accessories such as regulators, e.g., Medipure™ Heliox-LCQ System (PraxAir) and control box, e.g., Precision Control Flow (PraxAir). In some embodiments, a full delivery setup can be a device as described in, for example, Russian patent RU199823U1.

The term “heliox” as used herein refers to breathing gas mixtures of helium gas (He) and oxygen gas (O₂). In some embodiments, the heliox mixture can contain helium in the mixture of helium and oxygen at about 50%, 60%, 70%, 80% or 90% by volume, and contain oxygen in the mixture of helium and oxygen at about 50%, 40%, 30%, or 10% by volume, or any range therebetween. The heliox mixture can thus contain helium and oxygen in a volume ratio of 50:50, 60:40, 70:30, 80:20, 90:10, or any range therebetween. In some embodiments, heliox can generate less airway resistance through increased tendency to laminar flow and reduced resistance in turbulent flow.

The use of heat in heliox mixtures can further enhance drug delivery by increasing permeability of key physical barriers for drug absorption. Heating of mucosal surfaces can increase permeability by enhancing peripheral blood circulation and relaxing the interstitial junction, as well as other mechanisms. Helium has a thermal conductivity almost 10 times higher than oxygen and nitrogen and can facilitate heat transfer more efficiently. A dry heliox mixture can be used safely as a pretreatment step when warmed up to as high as 110° C., which can enable the dry heliox mixture to heat mucosal surfaces of the lung and respiratory tract more efficiently.

Various types of personal vaporizers are known in the art. In general, personal vaporizers are characterized by heating a solid drug or compound. Vaporizers can work by directly heating a solid drug or compound to a smoldering point. Vaporizing a solid or solid concentrate can be done by convection or conduction. Convection heating of solid concentrate involves a heating element coming into contact with water, or another liquid, which then vaporizes. The hot vapor in turn directly heats the solid or solid concentrate to a smoldering point, releasing a vapor to be inhaled by a user. Conduction heating involves direct contact between the solid or solid concentrate and the heating element, which brings the solid to a smoldering point, releasing vapor to be inhaled by a user. Though vaporizers present advantages over smoking in terms of lung damage, the drug/active agent that is vaporized can be substantially deteriorated by the vaporizing heat. In some embodiments, the pharmaceutically acceptable salts of the compound of Formula (I) are delivered via a nebulizer, which generates an aqueous-droplet aerosol, preferably a mist, containing the salt forms, which is optionally combined with a heated helium-oxygen mixture. In some embodiments, the salt forms of the disclosed compounds are delivered via a nebulizer, which generates an aqueous-droplet aerosol, preferably a mist, containing the salt forms, which is combined with a driving gas comprising nitrous oxide. The driving gas comprising nitrous oxide may be nitrous oxide gas itself or a therapeutic gas mixture, such as a N₂O—O₂ mixture or a N₂O -air mixture. The therapeutic gas mixture may further include other gases such as one or more of N₂, Ar, CO₂, Ne, CH₄, He, Kr, H₂, Xe, H₂O (e.g., vapor), etc. In some embodiments, the driving gas is a therapeutic gas mixture comprising N₂O , which is present at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture, or any range in between. The presence of nitrous oxide (being an NMDA receptor antagonist) in (or as) the driving gas can augment the effect of the disclosed compounds and provide the ability to use lower doses thereof to obtain similar levels of effect.

For example, a preparation of a pharmaceutically acceptable salt of the compound of Formula (I) can be placed into a liquid medium and put into an aerosol by a device, such as a nebulizer. In some embodiments, a nebulizer can be, for example, a pneumatic compressor nebulizer, an ultrasonic nebulizer, a vibrating mesh or horn nebulizer, or a microprocessor-controlled breath-actuated nebulizer. In some embodiments, a nebulizer device can be a device as described in, for example, Russian patent RU199823U1.

A nebulizer is a device that turns a drug, such as a pharmaceutically acceptable salt of the compound of Formula (I), in solution or suspension into a fine aerosol, such as a mist, for delivery to the lungs. A nebulizer can also be referred to as an atomizer. To atomize is to put a dissolved drug into an aerosol, such as a mist, form. To deliver a drug by nebulization, a drug can be dispersed in a liquid medium, for example, water, ethanol, or propylene glycol. Additionally, the salt forms of the disclosed compounds can be carried in a vehicle such as, for example liposomes, polymers, emulsions, micelles, nanoparticles, or polyethylenimine (PEI). Liquid drug formations for nebulizers can be, for example, aqueous solutions or viscous solutions. After application of a dispersing forcer (e.g., jet of gas, ultrasonic waves, or vibration of mesh), the dissolved drug is contained within liquid droplets, which are then inhaled. A mist can contain liquid droplets containing the drug in air or another gaseous mixture (e.g., a mixture of helium and oxygen).

Jet nebulizers (also known as pneumatic nebulizers or compressor nebulizers) use compressed gas to make a mist. In some embodiments, a jet nebulizer is a microprocessor-controlled breath-actuated nebulizer, also called a breath-actuated nebulizer. A breath-actuated nebulizer creates a mist only when a patient is inhaling, rather than creating a mist continuously. A mist can be generated by, for example, passing air flow through a Venturi in a nebulizer bowl or cup. A Venturi is a system for speeding the flow of a fluid by constricting fluid in a cone shape tube. In the restriction, the fluid must increase its velocity, thereby reducing its pressure and producing a partial vacuum. As the fluid exits the constriction point, its pressure increases back to the ambient or pipe level pressure. This can form a low-pressure zone that pulls up droplets through a feed tube from a solution of drug in a nebulizer bowl, and in turn this creates a stream of atomized droplets, which flow to a mouthpiece. Higher air flows lead to a decrease in particle size and an increase in output. Due to droplets and solvent that saturates the outgoing gas, jet nebulizers can cool a drug solution in the nebulizer and increase solute concentration in the residual volume. A baffle in a nebulizer bowl or cup can be impacted by larger particles, retaining them and returning them to the solution in the nebulizer bowl or cup to be reatomized. Entrainment of air through a nebulizer bowl as the subject inhales can increase mist output during inspiration. Generation of a mist can occur with a smaller particle size distribution, but using smaller particle sizes can result in an increased nebulization time.

The unit of measurement generally used for droplet size is mass median diameter (MMD), which is defined as the average droplet diameter by mass. This unit can also be referred to as the mass mean aerodynamic diameter, or MMAD. The MMD droplet size for jet nebulizers can be about 1.0, 1.5, 2.0, 2.5, 3.0, 3.5, 4.0, 5.0, 6.0, 7.0, 8.0, 9.0, 10.0 m or more (or any range between about 1.0 and 10.0 m), which can be smaller than that of ultrasonic nebulizers.

Ultrasonic nebulizers generate mists by using the vibration of a piezoelectric crystal, which converts alternating current to high-frequency (about 1 to about 3 MHz) acoustic energy. The solution breaks up into droplets at the surface, and the resulting mist is drawn out of the device by the patient's inhalation or pushed out by gas flow through the device generated by a small compressor. Ultrasonic nebulizers can include large-volume ultrasonic nebulizers and small-volume ultrasonic nebulizers. Droplet sizes tend to be larger with ultrasonic nebulizers than with jet nebulizers. The MMD droplet size for ultrasonic nebulizers can be about 2.0, 2.5, 3.0, 3.5, 4.0, 4.5, 5.0, 5.5, 6.0, 6.5, 7.0, 7.5, 8.0, 9.0, 10.0 m or more (or any range between about 2.0 and 10.0 m). Ultrasonic nebulizers can create a dense mist, with droplets at about 100, 150, 200, 250, 300 m/L or more.

Mesh nebulizer devices use the vibration of a piezoelectric crystal to indirectly generate a mist. Mesh nebulizers include, for example, active mesh nebulizers and passive mesh nebulizers. Active mesh nebulizers use a piezo element that contracts and expands on application of an electric current and vibrates a precisely drilled mesh in contact with the drug solution to generate a mist. The vibration of a piezoelectric crystal can be used to vibrate a thin metal plate perforated by several thousand holes. One side of the plate is in contact with the liquid to be atomized, and the vibration forces this liquid through the holes, generating a mist of tiny droplets. Passive mesh nebulizers use a transducer horn that induces passive vibrations in the perforated plate with tapered holes to produce a mist. Examples of active mesh nebulizers include the Aeroneb® (Aerogen, Galway, Ireland) and the eFlow® (PARI, Starnberg, Germany), while the Microair NE-U22 @(Omron, Bannockburn, IL) is a passive mesh nebulizer. Mesh nebulizers are precise and customizable. By altering the pore size of the mesh, the device can be tailored for use with drug solutions of different viscosities, and the output rate changed. Use of this method of atomization can offer several advantages. The size of the droplets can be extremely precise because droplet size can be determined by the size of the holes in the mesh (which may be tailor-made to suit the application). Nebulizer meshes can be manufactured using methods such as electrodeposition, electroplating, and laser cutting to produce a liquid particle in gas in the respirable range. Mesh can be made of metal alloy. The metals used in mesh manufacture can include platinum, palladium, nickel, and stainless steel. The size of the droplet is about twice the size of the mesh hole. Mesh holes, therefore, can be about 0.1, 0.5, 1.0, 1.5, 2.0, 2.5, 3.0, 3.5, 4.0, 4.5, 5.0 μm or more (or any value in between about 0.1 and 5.0 μm). Mist generation in mesh nebulizers can vary based on the shape of the mesh, the material that the mesh is made of, and also the way that the mesh is created. In other words, different meshes can produce different sized liquid particles suspended in gas. Generally, MMD droplet size for mesh nebulizers can be about 1.0, 1.5, 2.0, 2.5, 3.0, 3.5, 4.0, 4.5, 5.0, 5.5., 6.0, 6.1, 6.2, 6.3, 6.4, 6.5, 6.6, 6.7, 6.8, 6.9, 7.0 μm or more (or any value in between about 1.0 and 7.0 μm).

Additionally, droplet size can be programmable. In particular, geometric changes can be made to a nebulizer to provide a specific desired droplet size. Additionally, droplet size can be controlled independently of droplet velocity. The volume of liquid atomized, and the droplet velocity can also be precisely controlled by adjusting the frequency and amplitude of the mesh vibration. Furthermore, the number of holes in the mesh and their layout on the mesh can be tailored. Mesh nebulizers can be powered either by electricity or by battery.

A mist output rate in standing cloud mL per minute (for any atomization methodology described herein) can range from, for example, 0.1, 0.2. 0.3, 0.4, 0.5, 0.6, 0.7, 0.8, 0.9 mL/minute or more (or any range between about 0.1 and 0.9 mL/minute) and the residual volume in any type of nebulizer reservoir can range from a about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mL or more (or any range between about 0.01 and 2.0 mL). Precise droplet size control can be advantageous since droplet size can correlate directly to kinetic drug release (KDR). Precise control of KDR can be achievable with precise control of droplet size. Pharmaceutically acceptable salts of the compounds herein can be delivered via a mist using any methodology with an MMD droplet size of about 0.5, 1.0, 1.5, 2.0, 2.5, 3.0, 3.5, 4.0, 5.0, 6.0, 7.0, 8.0, 9.0, 10.0 m or more (or any range between about 0.5 and 10.0 m).

In some embodiments, a pharmaceutically acceptable salt of the compound of Formula (I) can be delivered via a continuous positive airway pressure (CPAP) or other pressure-assisted breathing device. A pressure-assisted breathing device forces a continuous column of compressed air or other gas at a fixed designated pressure against the face and nose of the patient, who is wearing a mask or nasal cap. When the patient's glottis opens to inhale, the pressure is transmitted throughout the airway, helping to open it. When the patient exhales, pressure from the deflating lungs and chest wall pushes air out against the continuous pressure, until the two pressures are equal. The air pressure in the airway at the end of exhalation is equal to the external air pressure of the machine, and this helps “splint” the airway open, allowing better oxygenation and airway recruitment. A pressure-assisted breathing device can be coupled with a means for introducing mist particles into the gas flow in the respiratory circuit and or a means for discontinuing the introduction of mist particles into the respiratory circuit when the patient exhales. See, e.g. U.S. Pat. No. 7,267,121.

In some embodiments, a mist can be delivered by a device such as a metered dose inhaler (MDI) (also referred to as a pressurized metered dose inhaler or pMDI), which generates an organic solvent-droplet mist containing the disclosed salt forms of compounds of Formula (I), which is optionally combined with a heated helium-oxygen mixture. In some embodiments, a pharmaceutically acceptable salt of the compound of Formula (I) can be delivered via a metered dose inhaler, MDI. MDI devices can include a canister which contains the pharmaceutically acceptable salt of the compound of Formula (I) and a propellant, a metering valve which dispenses the medicament from the canister, an actuator body that receives the canister and which forms an opening for oral inhalation, and an actuator stem which receives the drug from the canister and directs it out the opening in the actuator body. A non-limiting example of a metering valve and actuator is Bespak's BK357 valve and actuator (orfice d=0.22 mm) by Recipharm. Moving the drug canister relative to the actuator body and actuator stem causes the metering valve to release the predetermined amount of the drug. In some embodiments, the pharmaceutically acceptable salt of the compound of Formula (I) can be dissolved in a liquid propellant mixture (sometimes including small amounts of a volatile organic solvent) stored in a pressurized container of the MDI. The “metered dose” is the dose that is prepackaged in a single-dose inhaler, or which in a multidose inhaler is automatically measured out of a reservoir in preparation for inhalation. MDI devices can be aided with spacers. An MDI spacer is a spacer that goes between the MDI and the mouth of a user of the MDI. An MDI spacer allows droplets in the atomized dose to settle out a bit and mix with air or other gas, thus allowing for more effective delivery of a metered dose into a user's lungs when inhaled. An MDI spacer assists in preventing a user from inhaling the metered dose directly from an MDI where the dose would be traveling so fast that the droplets of the atomized spray from the MDI hit and stick to the back of the user's throat rather than being inhaled into the user's lungs where the drug of the metered dose is designed to be delivered. MDI devices offer the advantage of regular dosing, which can be controlled in the manufacture of the drug.

The pharmaceutically acceptable salt of the compound of Formula (I) can also be delivered by dry powder inhalers (DPI). In such DPI devices, the drug itself can form the powder or the powder can be formed from a pharmaceutically acceptable vehicle and the drug is releasably bound to a surface of the carrier powder such that upon inhalation, the moisture in the lungs releases the drug from the surface to make the drug available for systemic absorption. In some embodiments, the pharmaceutically acceptable salt of the compound of Formula (I) is delivered by use of a dry powder inhaler (DPI). Depending on the salt form used, the drug can be formed into the necessary powder itself (the pharmaceutically acceptable salt of the present disclosure is in solid particulate form), or can be releasably bound to a surface of a carrier powder. Such carrier powders are known in the art (see, e.g., H. Hamishehkar, et al., “The Role of Carrier in Dry Powder Inhaler”, Recent Advances in Novel Drug Carrier Systems, 2012, pp. 39-66). When the pharmaceutically acceptable salt of the compound of Formula (I) itself forms the dry powder, the selection of an appropriate physiologically acceptable salt form has increased importance, e.g., to prevent irritation of the pulmonary tract of the patient.

DPI is generally formulated as a powder mixture of coarse carrier particles and micronized drug particles with aerodynamic particle diameters of 1-5 m (see e.g., Iida, Kotaro, et al. “Preparation of dry powder inhalation by surface treatment of lactose carrier particles” Chemical and pharmaceutical bulletin 51.1 (2003): 1-5). Carrier particles are often used to improve drug particle flowability, thus improving dosing accuracy and minimizing the dose variability observed with drug formulations alone while making them easier to handle during manufacturing operations. Carrier particles should have several characteristics such as physico-chemical stability, biocompatibility and biodegradability, compatible with the drug substance and must be inert, available and economical. The choice of carrier particle (both content and size) is well within the purview of one of ordinary skill in the art. The most common carrier particles are made of lactose or other sugars, with a-lactose monohydrate being the most common lactose grade used in the inhalation field for such particulate carriers.

Any of the delivery devices above can be optionally manufactured with smart technology enabling remote activation of the drug delivery. The remote activation can be performed via computer or mobile app. To ensure security, the remote activation device can be password encoded. This technology enables a healthcare provider to perform telehealth sessions with a patient, during which the healthcare provider can remotely activate and administer the pharmaceutically acceptable salt of the compound of Formula (I) via the desired delivery device while supervising the patient on the televisit.

Delivery with Helium Oxygen Mixtures

In some embodiments, methods disclosed herein provide for systemic delivery of small doses of a pharmaceutically acceptable salt of the compound of Formula (I) or derivatives thereof. In particular, a pharmaceutically acceptable salt of the compound of Formula (I) or derivatives thereof can be delivered to a patient's CNS. Doses can be optimized for individual patients' metabolisms and treatment needs. Larger doses with deleterious or undesirable side-effects can be avoided by using small doses. Methods of treating various central nervous system (CNS) diseases and other conditions are described herein. The methods can comprise delivering a pharmaceutically acceptable salt of the compound of Formula (I) or derivative thereof to a patient in need thereof via inhalation of an aerosol comprising the drug and a carrier gas such as air, oxygen, helium, a mixture of helium and oxygen (i.e., a heliox mixture), other gases or other gas mixtures. In some embodiments, the carrier gas can be heated. The method can further comprise using a device containing a balloon with an oxygen-helium mixture equipped with a reducer and a mask connected to each other by a gas or air connecting tube, which contains an additional heating element capable of heating the gas mixture up to 120° C., a nebulizer with a vibrating porous plate or mesh, ensuring the passage of droplets with a size of less than 5 microns through it, and a disinfection unit.

In some embodiments a pharmaceutically acceptable salt of the compound of Formula (I) or derivative thereof is delivered to the lower respiratory tract, for instance, to a pulmonary compartment such as alveoli, alveolar ducts and/or bronchioles. From there, the drug can enter the blood stream and travel to the central nervous system. In some embodiments of the present disclosure, delivering a pharmaceutically acceptable salt of the compound of Formula (I) to a patient in need thereof via inhalation of a mist can deliver the compound of Formula (I) to the patient's CNS without passing through the liver. Administration via inhalation can allow gaseous drugs or those dispersed in a liquid or a mist, to rapidly deliver the compound of Formula (I) to the blood stream, bypassing first-pass metabolism. First-pass metabolism, also known as “first-pass effect” or “presystemic metabolism” describes drugs that enter the liver and undergo extensive biotransformation.

In some embodiments, the present disclosure provides a treatment step, in which a pharmaceutically acceptable salt of the compound of Formula (I) can be administered to a patient in need thereof by administering via inhalation a mixture of helium and oxygen heated to about 50° C., 51° C., 52° C., 53° C., 54° C., 55° C., 56° C., 57° C., 58° C., 59° C., 60° C., or more (or any range between 50° C. to 60° C.) and the atomized pharmaceutically acceptable salt of the compound of Formula (I). In some embodiments a mist or vapor of the pharmaceutically acceptable salt of the compound of Formula (I) can have a particle size from about 0.1 microns to about 10 microns (e.g., about 10, 5, 4, 3, 2, 1, 0.1 or less microns). In some embodiments, the pharmaceutically acceptable salt of the compound of Formula (I) can be atomized via a nebulizer creating an inhalant that is a mist. In some embodiments, the atomized pharmaceutically acceptable salt of the compound of Formula (I) is driven down the patient delivery line by the patient's inhalation. In some embodiments, the atomized pharmaceutically acceptable salt of the compound of Formula (I) is driven down the patient delivery line by the patient's inhalation using a carrier gas. The carrier gas can be air, oxygen, a mix of oxygen and helium, heated air, heated oxygen, a heated helium and oxygen mixture, among others.

In some embodiments, the treatment step can be preceded by a pretreatment step. In some embodiments, the pretreatment step can comprise first administering a pretreatment inhalation therapy prior to administration of the mist of the pharmaceutically acceptable salt of the compound of Formula (I). In some embodiments, the pretreatment inhalation step can comprise (i) administering via inhalation air, oxygen, or mixture of helium and oxygen heated to about 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., or more (or any range between about 90° C. and 120° C.) and no pharmaceutically acceptable salt of the compound of Formula (I), and then (ii) administering a treatment step of inhalation air, oxygen, a mix of oxygen and helium, heated air, heated oxygen, or heated helium and oxygen mixture. Heated air, heated oxygen, or heated helium and oxygen mixture, in combination with the atomized pharmaceutically acceptable salt of the compound of Formula (I) or derivative thereof, can be heated to about 50° C., 51° C., 52° C., 53° C., 54° C., 55° C., 56° C., 57° C., 58° C., 59° C., 60° C., or more (or any range between about 50° C. and 60° C.).

In some embodiments of the present disclosure, a pretreatment step (i) and a treatment step (ii) can be repeated 0, 1, 2, 3, 4, 5, or more times. In some embodiments of the present disclosure, steps (i) and (ii) can be repeated 0, 1, 2, 3, 4, 5, or more times followed by the treatment step, which can be repeated 0, 1, 2, 3, 4, 5, or more times. In some embodiments of the present disclosure, the treatment step can be repeated 0, 1, 2, 3, 4, 5, or more times with no pretreatment step.

Treatment, with optional pretreatment, can be administered once a week, twice a week, once a day, twice a day, three times a day or more, and other treatment regimens as set forth herein. Each treatment (i.e., inhalation session) can be for about 1, 5, 10, 20, 30, 45, 60 or more minutes.

A drug delivery procedure can comprise an inhaled priming no-drug hot heliox mixture to effectively preheat the mucosal bed followed by inhaling an atomized pharmaceutically acceptable salt of the compound of Formula (I), again driven by the heated heliox, but at lower temperatures, that are now dictated by lower heat tolerance to the wet vs. dry inhaled gas stream. Consequently, this procedure can be conducted in multiple repeated cycles, wherein a target PK and drug exposure is controlled by the concentration of the drug, temperature, flow rate of the helium oxygen mixture, composition of the mixture, number and durations of cycles, time and combinations of the above.

Methods of delivery described herein can be used to treat certain diseases and disorders, such as a central nervous system (CNS) disorder or psychological disorder, comprising administering via inhalation a heated mixture of helium and oxygen heated and an atomized pharmaceutically acceptable salt of the compound of Formula (I). The treatment can alleviate one or more symptoms of the disorder.

In some embodiments, the pharmaceutically acceptable salt of the compound of Formula (I) can be administered for treatment of CNS disease or other disorder. In some embodiments, the pharmaceutically acceptable salt of the compound of Formula (I) can be administered to treat depression including, but not limited to major depression, melancholic depression, atypical depression, or dysthymia. In some embodiments the pharmaceutically acceptable salt of the compound of Formula (I) can be administered to treat psychological disorders including anxiety disorder, obsessive compulsive disorder, addiction (narcotic addiction, tobacco addiction, opioid addiction), alcoholism, depression and anxiety (chronic or related to diagnosis of a life-threatening or terminal illness), compulsive behavior, or a related symptom.

In some embodiments, the disease or disorder can include central nervous system (CNS) disorders and/or psychological disorders, including, for example, post-traumatic stress disorder (PTSD), major depressive disorder (MDD), treatment-resistant depression (TRD), suicidal ideation, suicidal behavior, major depressive disorder with suicidal ideation or suicidal behavior, melancholic depression, atypical depression, dysthymia, non-suicidal self-injury disorder (NSSID), bipolar and related disorders (including, but not limited to, bipolar I disorder, bipolar II disorder, cyclothymic disorder), obsessive-compulsive disorder (OCD), generalized anxiety disorder (GAD), acute psychedelic crisis, social anxiety disorder, substance use disorders (including, but not limited to, alcohol use disorder, opioid use disorder, amphetamine use disorder, nicotine use disorder, and cocaine use disorder), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, chronic fatigue syndrome, Lyme disease, gambling disorder, eating disorders (including, but not limited to, anorexia nervosa, bulimia nervosa, binge-eating disorder, etc.), and paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido), and obesity. In some embodiments, the disease or disorder may include conditions of the autonomic nervous system (ANS). In some embodiments, the disease or disorder may include pulmonary disorders (e.g., asthma and chronic obstructive pulmonary disorder (COPD). In some embodiments, the disease or disorder may include cardiovascular disorders (e.g., atherosclerosis). Other diseases and disorders which may be treated are set forth herein.

The methods of administering a pharmaceutically acceptable salt of the compound of Formula (I) via inhalation, thereby delivering the compound of Formula (I) to the CNS (systemic drug delivery), such as through a nebulizer or other device as described herein (including, for example, using a heated helium-oxygen mixture), can lead to advantageous improvements in multiple PK parameters as compared to oral delivery. In particular, once administered, the compound of Formula (I) can cross the blood brain barrier and be delivered to the brain. As compared to oral delivery, the method of administering a pharmaceutically acceptable salt of the compound of Formula (I) to the patient via inhalation, such as with a nebulizer or other device as described herein, optionally with a heated heliox mixture, can increase bioavailability by at least 25% as compared to oral delivery. In some embodiments, the method of administering a pharmaceutically acceptable salt of the compound of Formula (I) to the patient via inhalation, such as with a nebulizer or other device as described herein, can increase bioavailability by about 10%, 25%, 30%, 35%, 40%, 50%, 55%, 60%, 65%, 70%, 80%, 85%, 90%, 95%, 99%, 99.9%, or more.

The method of administering a pharmaceutically acceptable salt of the compound of Formula (I) to the patient via nebulizer as described herein, can reduce Tₘₐₓ by at least 50% as compared to oral delivery. In some embodiments, the method of administering a pharmaceutically acceptable salt of the compound of Formula (I) to the patient via nebulizer as described herein, can reduce Tₘₐₓ by at 30%, 40%, 50%, 55%, 60%, 65%, 70%, 80%, 85%, 90%, 95%, 99%, 99.9%, or more. In some embodiments, the method of administering a pharmaceutically acceptable salt of the compound of Formula (I) to the patient via nebulizer or other device as described herein, can increase Cₘₐₓ by at least 25% as compared to oral delivery. In some embodiments, the method of administering a pharmaceutically acceptable salt of the compound of Formula (I) to the patient via nebulizer or other device as described herein, can increase Cₘₐₓ by about 10%, 25%, 30%, 35%, 40%, 50%, 55%, 60%, 65%, 70%, 80%, 85%, 90%, 95%, 99%, 99.9%, or more. Furthermore, a method of administering a pharmaceutically acceptable salt of the compound of Formula (I) to the patient via inhalation using a nebulizer or other device as described herein, can allow clinical protocols enabling dose titration and more controlled exposure. Controlled exposure enables adjusting the patient experience and providing overall improved therapeutic outcomes. With the smart technology enabled devices for inhalation delivery noted above, the dose titration and controlled delivery can be performed remotely by the healthcare worker, enabling the patient to be in the comfort of their own home, improving the patient's experience and outcome.

In some embodiments, a system is provided for administering the pharmaceutically acceptable salt of the compound of Formula (I) that includes a container comprising a solution of a pharmaceutically acceptable salt of the compound of Formula (I) and a nebulizer physically coupled or co-packaged with the container and adapted to produce an aerosol, preferably a mist, of the solution having a particle size from about 0.1 microns to about 10 microns (e.g., about 10, 5, 4, 3, 2, 1, 0.1 or less microns).

Combination of 5-HT_{2A} receptor agonist and an NMDA receptor antagonist

The present disclosure is also directed to combination drug therapies based on administration of both a salt form of a compound of Formula (I) (as a 5-HT_{2A} receptor agonist) and a N-methyl-D-aspartate (NMDA) receptor antagonist. It has been found that the combination drug therapy shows enhanced activity and improved patient experience when treating diseases or disorders associated with 5-HT_{2A} and/or NMDA receptors (e.g., a neuropsychiatric disease or disorder, a central nervous system (CNS) disorder and/or a psychological disorder), for example, by providing therapeutic efficacy while reducing or eliminating psychiatric adverse effects such as acute psychedelic crisis (bad trip) and dissociative effects from hallucinogens (out of body experience).

Non-limiting examples of NMDA receptor antagonist suitable for use in the combination drug delivery methods disclosed herein may include, but are not limited to, ketamine, nitrous oxide, memantine, amantadine, dextromethorphan (DXM), phencyclidine (PCP), methoxetamine (MXE), dizocilpine (MK-801), esmethadone, or a combination thereof. In particular, nitrous oxide (N₂O), commonly known as laughing gas, is a rapid and effective analgesic gas that has a fast onset and rarely produces side effects when administered under proper medical supervision. Nitrous oxide is also a dissociative inhalant known to cause euphoria during inhalation. Prominent effects of nitrous oxide are increased feelings of euphoria, a heightened pain threshold, and involuntary laughing. Furthermore, unlike ketamine, nitrous oxide is not addictive. For these reasons, the use of nitrous oxide as the NMDA receptor antagonist is preferred.

In some embodiments, the combination therapy involves providing the salt form of the compound of Formula (I) (as the 5-HT_{2A} receptor agonist) and the NMDA receptor antagonist as a single dosage form for administration to a patient (e.g., each is combined to provide a single aerosol that is inhaled by the patient). For example, when the NMDA receptor antagonist is nitrous oxide, the pharmaceutically acceptable salt of the compound of Formula (I) may be present in the liquid phase of the aerosol, while the nitrous oxide may be present in the gas phase of the aerosol. The nitrous oxide (or therapeutic gas mixture comprising nitrous oxide) may be used in the generation of the aerosol or as a carrier gas used to deliver a generated aerosol to the patient. When a generated aerosol is combined with a carrier gas, the carrier gas becomes a part of the gas phase of the aerosol, i.e., the liquid phase of the aerosol becomes entrained in/diluted by the carrier gas. In some embodiments, the combination therapy involves providing the pharmaceutically acceptable salt of the compound of Formula (I) and the NMDA receptor antagonist as separate dosage forms. For example, the salt form of the compound of Formula (I) may be provided as an aerosol, preferably a mist, while the NMDA receptor antagonist is provided separately as a therapeutic gas mixture. Alternatively, the pharmaceutically acceptable salt of the compound of Formula (I) may be provided as an injectable (e.g., intravenous, intradermal, subcutaneous, intramuscular, etc.), bolus, infusion, perfusion, etc., while the NMDA receptor antagonist is provided as a therapeutic gas mixture for inhalation delivery.

The co-action of the salt form of a compound of Formula (I) and a NMDA receptor antagonist (e.g., nitrous oxide) may provide multiple benefits. For example, the NMDA receptor antagonist may control and/or reduce the activating effects of the 5-HT₂Rs, thereby reducing the risk of overstimulation and occurrences of psychiatric adverse effects such as acute psychedelic crisis. Additionally, administration of the NMDA receptor antagonist may enable the use of a reduced therapeutic dose of the HT_{2A} receptor agonist (e.g., the pharmaceutically acceptable salt of the compound of Formula (I)), thereby decreasing the likelihood of a negative patient experience. Similarly, administration of the pharmaceutically acceptable salt of a compound of Formula (I) may reduce the amount of NMDA receptor antagonist necessary for a therapeutic effect, which in the case of nitrous oxide may alleviate certain side effects such as induced involuntary laughter and the general feelings of anxiety associated therewith. Thus, it is believed that co-administration would reduce the likelihood of a negative experience from the psychedelic administration, either because less psychedelic would be administered or the NMDA receptor antagonist (e.g., nitrous oxide, ketamine, etc.) would enable more efficient functioning of the psychedelic. Similarly, such co-administration would reduce the time or amount of NMDA receptor antagonist (e.g., nitrous oxide, ketamine, etc.) necessary for a therapeutic effect.

NMDA receptor antagonists (e.g., nitrous oxide) and 5-HT_{2A} receptor agonists function via different pharmacological pathways. However, both pathways appear to ultimately converge in a cascade at mTOR (mammalian target of rapamycin, or mechanistic target of rapamycin). Thus, a shared mechanism of action appears to exist between NMDA receptor antagonists and tryptamine psychedelics. Specifically, mTOR's signaling pathway may be modulated by 5-HT_{2A} receptor activation and NMDA antagonism. Without being bound by theory, such modulation of the mTOR pathway may underpin the immediate and long-lasting therapeutic and synergistic benefits of combined administration of both agents. As such, in some embodiments, administration of both agents at sub-psychedelic doses enables therapeutic efficacy without a psychedelic experience.

In addition, it has been found that atrophy of neurons in the prefrontal cortex (PFC) plays a key role in the pathophysiology of depression and related disorders, neurological and neurodegenerative disorders, and other diseases or disorders disclosed herein which are associated with neuroplastic changes, such as those associated with suppressed neurogenesis or maladaptive neuroplasticity. The ability to promote both structural and functional plasticity in the PFC has been hypothesized to underlie the fast-acting antidepressant properties of the dissociative anesthetic ketamine but also the long-lasting effect after a single administration. Without being bound by theory, it is believed that the combination drug therapy disclosed herein may function by synergistically increasing neuritogenesis and spinogenesis, including increased density of dendritic spines, thereby providing or contributing to long-lasting therapeutic benefits.

Indeed, the combination drug therapy disclosed herein is designed to be synergistic as both NMDA receptor antagonism (e.g., as brought about through nitrous oxide administration) and 5-HT_{2A} receptor agonist administration activate neuroplasticity, thereby achieving a significant therapeutic enhancement effect compared to administration of either agent, NMDA receptor antagonist or 5-HT_{2A} receptor agonist, alone.

It should be understood that a ratio of the compound of Formula (I) (provided in salt form) and the NMDA receptor antagonist (e.g., nitrous oxide) given to any particular patient will depend upon a variety of factors, such as the activity of the specific compounds employed, the age, sex, general health of the patient, time of administration, rate of excretion, and the type and severity of the particular disease or condition being treated. In some embodiments, a weight ratio of the compound of Formula (I) and the NMDA receptor antagonist administered to the patient may range from about 1:100 to about 100:1, or any range therebetween, e.g., from about 1:75, from about 1:50, from about 1:40, from about 1:30, from about 1:20, from about 1:10, from about 1:8, from about 1:6, from about 1:5, from about 1:4, from about 1:3, from about 1:2, from about 2:3, from about 1:1, and up to about 100:1, up to about 75:1, up to about 50:1, up to about 40:1, up to about 30:1, up to about 20:1, up to about 10:1, up to about 8:1, up to about 6:1, up to about 5:1, up to about 4:1, up to about 3:1, up to about 2:1. Ratios outside of this range may also be employed, in certain circumstances.

The combination drug therapy is intended to embrace administration of the salt form of a compound of Formula (I) and a NMDA receptor antagonist (e.g., nitrous oxide) in a sequential manner, that is, wherein each therapeutic agent is administered at a different time, as well as administration of these therapeutic agents, or at least two of the therapeutic agents, in a substantially simultaneous manner (e.g., co-administration). Substantially simultaneous administration can be accomplished, for example, by administering to the subject a single dosage form having a fixed ratio of each therapeutic agent or in multiple, single dosage forms for each of the therapeutic agents. Administration of the salt form of a compound of Formula (I) and a NMDA receptor antagonist (e.g., nitrous oxide), whether in a single dosage form or separate dosage forms, can be carried out by any administration route set forth herein. In some embodiments, both the salt form of a compound of Formula (I) and the NMDA receptor antagonist are administered via inhalation, preferably in aerosol (e.g., mist) form. In some embodiments, the salt form of a compound of Formula (I) is administered intravenously (IV), and the NMDA receptor antagonist is administered via inhalation. In some embodiments, the salt form of a compound of Formula (I) is administered subcutaneously, and the NMDA receptor antagonist is administered via inhalation. In some embodiments, the salt form of a compound of Formula (I) is administered intramuscularly, and the NMDA receptor antagonist is administered via inhalation. In some embodiments, the salt form of a compound of Formula (I) is administered intranasally, and the NMDA receptor antagonist is administered via inhalation. In some embodiments, the salt form of a compound of Formula (I) is administered orally, and the NMDA receptor antagonist is administered via inhalation. In some embodiments, both the salt form of a compound of Formula (I) and the NMDA receptor antagonist are administered transdermally or subcutaneously. The compositions for inhalation such as pharmaceutically acceptable vehicles/carriers, etc. for the single or separate dosage forms are set forth herein.

When the pharmaceutically acceptable salt of the compound of Formula (I) and the NMDA receptor antagonist (e.g., nitrous oxide) are administered sequentially (i.e., separately), the interval of time between administration of the therapeutic agents may range from about 5 seconds to about a week or longer, or any time in between, e.g., about 5 seconds, about 10 seconds, about 20 seconds, about 30 seconds, about 1 minute, about 2 minutes, about 3 minutes, about 4 minutes, about 5 minutes, about 10 minutes, about 20 minutes, about 30 minutes, about 40 minutes, about 50 minutes, about 1 hour, about 2 hours, about 3 hours, about 4 hours, about 5 hours, about 8 hours, about 10 hours, about 12 hours, about 18 hours, about 1 day, about 2 days, about 3 days, about 4 days, about 5 days, about 6 days, about 1 week. For sequential administration, the composition containing the pharmaceutically acceptable salt of the compound of Formula (I) and the composition containing the NMDA receptor antagonist are preferably administered from about 5 seconds to less than 1 minute, less than 2 minutes, less than 2 minutes, less than 3 minutes, less than 4 minutes, less than 5 minutes, less than 10 minutes, less than 15 minutes, less than 30 minutes, less than 45 minutes, less than 1 hour, less than 2 hours, or less than 4 hours apart. When administered substantially simultaneously, the pharmaceutically acceptable salt of the compound of Formula (I) and the NMDA receptor antagonist (e.g., nitrous oxide) are administered within 5 seconds, within 4 seconds, within 3 seconds, within 2 seconds, within 1 second of each other, including at the same time (e.g., when administered within the same dosage form, such as in the same aerosol).

In some embodiments, the NMDA receptor antagonist used in the combination drug therapy is nitrous oxide. Nitrous oxide may be administered alone, or as a therapeutic gas mixture, e.g., N₂O and O₂; N₂O and air; N₂O and medical air (medical air being 78% nitrogen, 21% oxygen, 1% other gases); N₂O and a N₂/O₂ mix; N₂O and O₂ enriched medical air; N₂O and a He/O₂ mix etc. Thus, in addition to nitrous oxide and oxygen, the therapeutic gas mixture may further include other gases such as one or more of N₂, Ar, CO₂, Ne, CH₄, He, Kr, H₂, Xe, H₂O (e.g., vapor), etc. For example, nitrous oxide may be administered using a blending system that combines N₂O , O₂ and optionally other gases from separate compressed gas cylinders into a therapeutic gas mixture which is delivered to a patient via inhalation. Alternatively, the therapeutic gas mixture containing nitrous oxide may be packaged, for example, in a pressurized tank or in small, pressurized canisters which are easy to use and/or portable. The blending system and/or pressurized tanks/canisters may be adapted to fluidly connect to an inhalation device such as a device capable of generating an aerosol of the pharmaceutically acceptable salt of the compound of Formula (I). Nitrous oxide itself, or the therapeutic gas mixture comprising nitrous oxide may be used for the generation of the aerosol (i.e., as the gas phase component of the aerosol) or as a carrier gas to facilitate the transfer of a generated aerosol to a patient's lungs. In some embodiments, N₂O is present in the therapeutic gas mixture at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture. The therapeutic gas mixture containing nitrous oxide can be administered over any desired duration, e.g., 5 minutes, 10 minutes 15 minutes, 20 minutes, 30 minutes, 40 minutes, 45 minutes, 50 minutes, 60 minutes, 90 minutes, 120 minutes, 150 minutes, 180 minutes, or any range therebetween.

Previously, mixtures of nitrous oxide and oxygen have been proposed to treat MDD and TRD (see, e.g., Nagele, P. et al. Biol. Psych. 2015 and Nagele, P. et al. Science Transl. Med., 2021), showing efficacy at 50/50 mixtures and 25/75 mixtures of nitrous oxide/oxygen, with 1 hour treatment regimens. The present inventors have found, however, that lower levels of nitrous oxide, for the same time period or less, can provide similar efficacy but with a significantly reduced side effect profile. Thus, in some embodiments, N₂O is administered in a therapeutic gas mixture, substantially simultaneously with, or in some instances sequentially with (separately from), the salt form of the compound of Formula (I), at a concentration ranging from 15 vol %, from 16 vol %, from 17 vol %, from 18 vol %, from 19 vol %, and up to 25 vol %, up to 24 vol %, up to 23 vol %, up to 22 vol %, up to 21 vol %, up to 20 vol %, relative to a total volume of the therapeutic gas mixture.

Fast-acting combination drug therapies can also be selected through selection of 5-HT_{2A} receptor agonists (e.g., a salt form of a compound of Formula (I)) with a short elimination half-life (t_{1/2}) and selection of a fast-acting NMDA receptor antagonist such as nitrous oxide. In some embodiments, a salt form of a compound of Formula (I) is selected which has an elimination half-life (t_{1/2}) of less than 2 hours, e.g., from about 15 minutes to less than about 120 minutes, less than about 110 minutes, less than about 100 minutes, less than about 90 minutes, less than about 80 minutes, less than about 70 minutes, less than about 60 minutes, less than about 50 minutes, less than about 40 minutes, less than about 30 minutes, less than about 20 minutes. Regarding the fast-acting NMDA receptor antagonist, nitrous oxide, in particular, gives a rapid onset of effects yet is quickly removed from the body-its effects cease almost immediately upon removal e.g., when the flow of gas is stopped. Thus, when fast acting combination drug therapies are desired, nitrous oxide is well suited for administration with a fast-acting and short lived 5-HT_{2A} receptor agonist. The aforementioned fast-acting therapeutic combination may be advantageous for acute treatment applications, such as to treat acute psychiatric conditions e.g., as a rescue medicine when someone is suicidal. The therapeutic combination may be especially useful to treat acute conditions that require a quick onset of effect, a short duration of action and minimal psychiatric adverse effects. Non-limiting examples of acute psychiatric conditions include, but are not limited to, suicidal ideation and suicide attempts, social anxiety disorder, drug withdrawal, post-traumatic stress disorder (PTSD), and panic attacks.

In some embodiments, the pharmaceutically acceptable salt of the compound of Formula (I) and the NMDA receptor antagonist (e.g., nitrous oxide) are each delivered by aerosol inhalation, as a single dosage form or as separate dosage forms. The aerosol, preferably a mist, may be generated by any capable device (e.g., a pressurized container, pump, spray, atomizer, or nebulizer), such as those devices disclosed herein, with or without the use of a propellant. When nitrous oxide is administered concurrently with the pharmaceutically acceptable salt of the compound of Formula (I), the nitrous oxide may dually act as a carrier gas or propellant for the aerosol generation and as a therapeutic agent (an NMDA receptor antagonist).

In some embodiments, the delivery device is an inhalation delivery device for delivery of a combination of nitrous oxide and a pharmaceutically acceptable salt of a compound of Formula (I) by inhalation to a patient in need thereof, comprising an inhalation outlet portal for administration of the combination to the patient; a container configured to deliver nitrous oxide gas to the inhalation outlet portal; and a device configured to generate and deliver an aerosol comprising the pharmaceutically acceptable salt of a compound of Formula (I) to the inhalation outlet portal. In some embodiments, the inhalation outlet portal is selected from a mouthpiece or a mask covering the patient's nose and mouth. In some embodiments, the device configured to generate and deliver the aerosol to the inhalation outlet portal is a nebulizer. In some embodiments, the nebulizer is a jet nebulizer and the nitrous oxide gas, alone, or in combination with other gases (therapeutic gas mixture containing nitrous oxide), acts as a driving gas for the jet nebulizer. Accordingly, nitrous oxide delivered using a nebulizer (e.g., jet nebulizer) may dually act as a therapeutic agent and as a driving gas to entrain the nebulized form of the pharmaceutically acceptable salt of the compound of Formula (I). In some embodiments, the device further comprises smart technology, e.g., electronics, configured to provide remote activation and operational control of the inhalation delivery device as noted above.

In some embodiments, the device is a dual delivery device configured to administer the pharmaceutically acceptable salt of the compound of Formula (I), preferably in the form of an aerosol, and to simultaneously administer a controlled amount of nitrous oxide. Any of the above aerosol delivery devices can be used for such a device, with the addition of a source of nitrous oxide configured to provide a metered, controlled dose/flow rate of nitrous oxide through the same administration outlet as the aerosol delivery device. In some embodiments, the driving gas for the nebulization of the pharmaceutically acceptable salt of the compound of Formula (I) is the nitrous oxide itself.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### I. Synthetic Procedures

### Examples 1-9

Examples 1-9 were prepared by crystallization of the free base of 2-(1H-indol-3-yl)-N,N-dimethylethan-1-amine (I-1) with stoichiometric (1.0 molar equivalent) quantities, or with sub-stoichiometric (0.5 molar equivalents) quantities in the case of hemi-salts, of the corresponding organic acid (fumaric acid, benzoic acid, salicylic acid, succinic acid, oxalic acid, or glycolic acid), from ethanol. The salt form identifier and salt type are provided in Table 3.

TABLE 3
Examples 1-9

| Example No. | Salt form identifier | Salt type of compound |
|---|---|---|
| 1 | I-1a | Fumarate of I-1 |
| 2 | I-1b | Benzoate of I-1 |
| 3 | I-1c | Salicylate of I-1 |
| 4 | I-1d | Succinate of I-1 |
| 5 | I-1e | Oxalate of I-1 |
| 6 | I-1f | Glycolate of I-1 |
| 7 | I-1g | Hemi-oxalate of I-1 |
| 8 | I-1h | Hemi-fumarate of I-1^{a} |
| 9 | I-1i | Hemi-fumarate of I-1^{a} |

^{a}Two crystalline polymorphs of the hemi-fumarate salt were isolated

### Examples 10-13

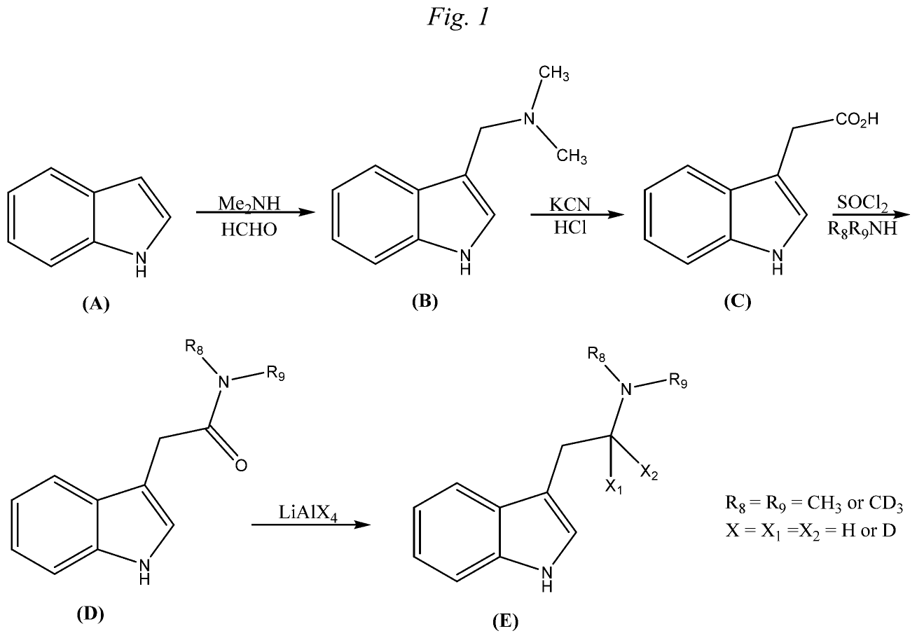

Synthesis of 2-(1H-indol-3-yl)-N,N-dimethylethan-1-amine-1,1-d₂ (I-2) was carried out according to FIG. 1. Indole (A) was iminoformylated using formaldehyde/dimethylamine to produce intermediate (B), which was then converted to the 3-acetic acid intermediate (C) using potassium cyanide in HCl. Subsequent treatment with thionyl chloride and dimethylamine (R₈=R₉=CH₃) produced amide (D, R₈=R9=CH₃), that was reduced by LiAlD₄ to yield compound I-2. The structure of the product was confirmed by ¹H NMR.

Examples 10-13 are prepared by crystallization of the free base of I-2 with stoichiometric (1.0 molar equivalent) quantities of the corresponding organic acid (fumaric acid, benzoic acid, salicylic acid, or succinic acid) from ethanol. The salt form identifier and salt type are provided in Table 4.

TABLE 4
Examples 10-13

| Example No. | Salt form identifier | Salt type of compound |
|---|---|---|
| 10 | I-2a | Fumarate of I-2 |
| 11 | I-2b | Benzoate of I-2 |
| 12 | I-2c | Salicylate of I-2 |
| 13 | I-2d | Succinate of I-2 |

### Examples 14-17

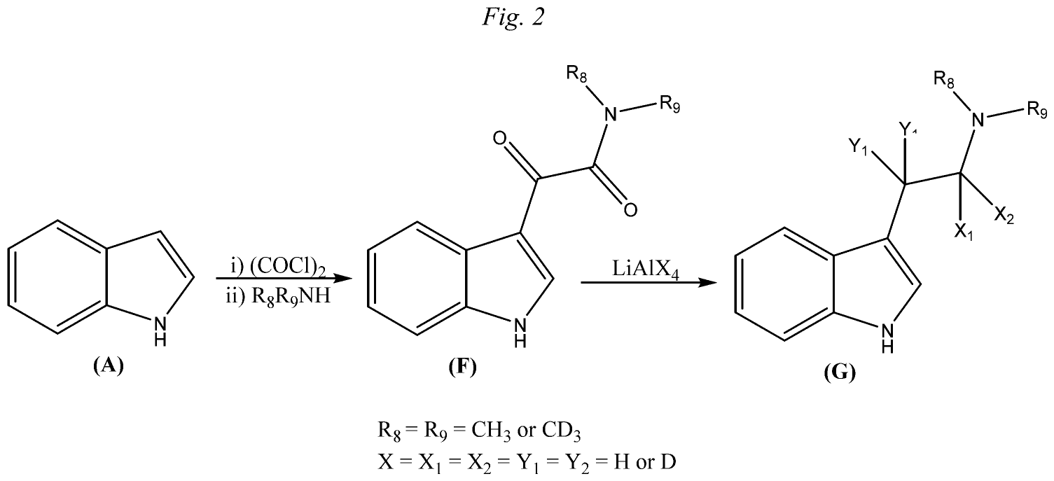

Synthesis of 2-(1H-indol-3-yl)-N,N-bis(methyl-d₃)ethan-1-amine (I-4) is carried out according to FIG. 2. Indole (A) is acylated with oxalyl chloride and is then reacted with dimethyl-d6-amine to produce amide (F, R₈=R₉=CD₃). Subsequent reduction with LiAlH₄ yields compound I-4. The structure of the product will be confirmed by ¹H NMR.

Examples 14-17 are prepared by crystallization of the free base of I-4 with stoichiometric (1.0 molar equivalent) quantities of the corresponding organic acid (fumaric acid, benzoic acid, salicylic acid, or succinic acid) from ethanol. The salt form identifier and salt type are provided in Table 5.

TABLE 5
Examples 14-17

| Example No. | Salt form identifier | Salt type of compound |
|---|---|---|
| 14 | I-4a | Fumarate of I-4 |
| 15 | I-4b | Benzoate of I-4 |
| 16 | I-4c | Salicylate of I-4 |
| 17 | I-4d | Succinate of I-4 |

### Examples 18-21

Synthesis of 2-(1H-indol-3-yl)-N,N-dimethylethan-1-amine-1,1,2,2-d₄ (I-5) was carried out according to FIG. 2. Indole (A) was acylated with oxalyl chloride and then reacted with dimethylamine to produce amide (F, R₈=R₉=CH₃). Subsequent reduction with LiAlD₄ yielded compound I-5. The structure of the product was confirmed by ¹H NMR.

Examples 18-21 are prepared by crystallization of the free base of I-5 with stoichiometric (1.0 molar equivalent) quantities of the corresponding organic acid (fumaric acid, benzoic acid, salicylic acid, or succinic acid) from ethanol. The salt form identifier and salt type are provided in Table 6.

TABLE 6
Examples 18-21

| Example No. | Salt form identifier | Salt type of compound |
|---|---|---|
| 18 | I-5a | Fumarate of I-5 |
| 19 | I-5b | Benzoate of I-5 |
| 20 | I-5c | Salicylate of I-5 |
| 21 | I-5d | Succinate of I-5 |

### Examples 22-25

Synthesis of 2-(1H-indol-3-yl)-N,N-bis(methyl-d₃)ethan-1-amine-1,1-d₂ (I-6) was carried out according to FIG. 1. Indole (A) was iminoformylated using formaldehyde/dimethylamine to produce intermediate (B), which was then converted to the 3-acetic acid intermediate (C) using potassium cyanide in HCl. Subsequent treatment with thionyl chloride and dimethyl-d6-amine (R₈=R₉=CD₃) produced amide (D, R₈=R₉=CD₃), that was reduced by LiAlD₄ to yield compound I-6. The structure of the product was confirmed by ¹H NMR.

Examples 22-25 are prepared by crystallization of the free base of I-6 with stoichiometric (1.0 molar equivalent) quantities of the corresponding organic acid (fumaric acid, benzoic acid, salicylic acid, or succinic acid) from ethanol. The salt form identifier and salt type are provided in Table 7.

TABLE 7
Examples 22-25

| Example No. | Salt form identifier | Salt type of compound |
|---|---|---|
| 22 | I-6a | Fumarate of I-6 |
| 23 | I-6b | Benzoate of I-6 |
| 24 | I-6c | Salicylate of I-6 |
| 25 | I-6d | Succinate of I-6 |

### Examples 26-29

Synthesis of 2-(1H-indol-3-yl)-N,N-bis(methyl-d₃)ethan-1-amine-1,1,2,2-d₄ (I-8) was carried out according to FIG. 2. Indole (A) was acylated with oxalyl chloride and then reacted with dimethyl-d₆-amine to produce amide F (R₈=R₉=CD₃). Amide F (R₈=R₉=CD₃) (7.04 μg, 31.67 mmol) was charged to a flask under a nitrogen atmosphere. 2-MeTHF (140 ml, 20 vol) was then charged and stirred at room temperature for 15 minutes, though not all solid dissolved fully in this time. Lithium aluminum deuteride (LiAlD₄)(4.04 μg, 96.23 mmol, 3.0 eq) was charged in portions over 45 minutes, maintaining the temperature of the reaction mixture <40° C. After complete addition, the reaction mixture was heated to 65° C. for 16 hours. The batch was cooled to room temperature and completion confirmed by HPLC analysis. A water/THF mixture (35 ml, 5 vol, 1:9) was added dropwise over 90 minutes, maintaining the temperature <30° C. 15% NaOH (aq) (3.5 ml, 0.5 vol) was then added dropwise, followed by further water (10.5 ml, 1.5 vol). This mixture was stirred at room temperature for 30 minutes then filtered and the solids washed with 2-MeTHF (3×70 ml, 3×10 vol), slurrying them each time before deliquoring. The liquors were concentrated to dryness, leaving compound I-8 as a viscous orange-red oil. The structure of the product was confirmed by ¹H NMR. Compound I-8 was then taken up in ethanol (49 ml, 7 vol), and this I-8 ethanol solution was used in the salt formation experiments provided below. The salt form identifier and salt type are provided in Table 8.

TABLE 8
Examples 26-29

| Example No. | Salt form identifier | Salt type of compound |
|---|---|---|
| 26 | I-8a | Fumarate of I-8 |
| 27 | I-8b | Benzoate of I-8 |
| 28 | I-8c | Salicylate of I-8 |
| 29 | I-8d | Succinate of I-8 |

Example 26. A flask was charged with 15 ml of I-8 ethanol solution which was heated to reflux, and a single charge of fumaric acid (1.07 μg, 1.05 eq) added. After complete dissolution, this was allowed to cool to room temperature, then cooled further to 0° C. and filtered, with additional ethanol (3×5 ml, 3×2 vol) used to rinse out the flask and wash the cake. I-8a was isolated as a crystalline solid with a yield of 1.480 g (4.71 mmol, 44.6%, 97.0% by LC, ¹H NMR confirmed identity as 1:1 fumarate salt).

Example 27. A flask was charged with 18 ml of I-8 ethanol solution which was heated to reflux, and then benzoic acid (4.04 μg, 3.15 eq) was added in one charge. After ensuring all solid had dissolved, the solution was cooled in an ice bath and stirred for an additional 60 minutes at this temperature then filtered and further ice cold ethanol (2×2 vol) used to rinse out the flask and wash the cake. 2.272 g (7.09 mmol) of I-8b was obtained. Due to residual impurities observed in the HPLC analysis of this compound, it was slurried in ethanol (5 ml, 2 vol) for 16 hours. It was then cooled to 0° C. and filtered, with further ethanol (5 ml, 2 vol) used to rinse out the flask and wash the solids. However, the solids were amorphous and did not have a crystalline form. They were therefore dissolved in ethanol (15 ml, 7.5 vol) at reflux, cooled to 0° C. and filtered, using ethanol (5 ml, 2 vol) to wash them. The isolated I-8b was thus obtained as a white crystalline solid in a yield of 1.534 g (4.79 mmol, 45.4%, 91.1% by LC, ¹H NMR confirmed identity as 1:1 benzoate salt).

Example 28. A flask was charged with 18 ml of I-8 ethanol solution which was heated to reflux, and further ethanol (11.7 ml, total 12 vol) and salicylic acid (1.52 μg, 1.05 eq) was added as a single charge. Once fully dissolved, the solution was cooled to 0° C. The resulting solids were filtered and washed with ice cold ethanol (2×2 vol), providing I-8c with a yield of 2.860 g (8.50 mmol). Due to residual impurities observed in the HPLC analysis of this compound, it was slurried in ethanol (5 ml, 2 vol) for 16 hours. It was then cooled to 0° C. and filtered, with further ethanol (5 ml, 2 vol) used to rinse out the flask and wash the solids. However, the solids were amorphous and did not have a crystalline form. They were therefore dissolved in ethanol (60 ml, 30 vol) at reflux, cooled to 0° C. and filtered, using ethanol (5 ml, 2 vol) to wash them. The isolated I-8c was thus obtained as a white crystalline solid in a yield of 2.311 g (6.87 mmol, 65.1%, 91.5% by LC, ¹H NMR confirmed identity as 1:1 salicylate salt).

Example 29 is prepared analogously by crystallization of the free base of I-8 with a stoichiometric (1.0 molar equivalent) quantity of succinic acid from ethanol.

### II. Analyses

Samples were analyzed in detail using differential scanning calorimetry (DSC), X-ray powder diffraction (XRPD), dynamic vapor sorption (DVS), and ¹H nuclear magnetic resonance (NMR) spectroscopy, using the experimental details listed below.

### Differential Scanning Calorimetry (DSC)

All DSC curves were acquired using a Mettler Toledo 823 calorimeter, interfaced with a TA8000 workstation operating Mettler Toledo Stare software version 9.01. Typical analysis conditions were as follows:
Start temperature; 20° C.
Heating rate; 10° C. min⁻¹
End temperature; 320° C.
Purge gas; Nitrogen at 70 mL·min⁻¹
Sample pan; 40 L Aluminum pan with punctured lid

4-7 mg of sample was packed into an aluminum sample pan. The instrument was calibrated using traceable standards of indium, water and cyclohexane with respect to temperature and heat flow, prior to making any measurements.

### X-Ray Powder Diffraction (XRPD)

X-ray powder diffractograms were acquired using a Bruker D5000 diffractometer in Bragg-Brentano configuration. Extended acquisition parameters were employed for each batch of the drug substance, as detailed below:
Source; CuKα
Wavelength; 1.5406 Å
Step range; 2-40° (2θ)
Step size; 0.01°(2θ)
Time per step; 4.0 s
Divergence slit width; 2 mm
Antiscatter slit width; 2 mm
Detector slit width; 0.2 mm
Sample rotation; Engaged
Tube accelerating potential; 40 kV
Tube accelerating current; 30 mA
Temperature; Ambient (nominally 18-22° C.)

Approximately 2-5 mg of each sample was mounted on a silicon base, and a flat surface was created using a glass slide, for the analysis. All data was smoothed by the use of Fourier algorithms and the background was subtracted from each diffractogram.

Instrument performance checks were completed prior to measurements using a NIST traceable standard of corundum and also using a standard of Arkansas stone quartz. The Arkansas stone quartz is a recognized standard provided by the instrument manufacturers but does not have a batch number. Its use is continued since a database has been created with the standard to monitor performance. However, the parameters such as diffraction angle accuracy, resolution and sensitivity were all assessed using the traceable corundum standard.

### Dynamic Vapor Sorption (DVS)

Dynamic vapor sorption-desorption experiments were carried out using a DVS Resolution instrument supplied by Surface Measurement Systems. Data were acquired using the following acquisition parameters:
Solvent; Water
Start relative humidity; 30% RH
Relative humidity cycle (% RH); 30, 40, 50, 60, 70, 80, 90, 95, 90, 80, 70, 60, 50, 40, 30, 20, 10, 0, 10, 20, 30
Equilibrium condition (dm/dt); 0.002%/minute
Equilibrium condition window; 10 minutes
Minimum stage period; 5 minutes
Maximum stage period; 360 minutes
Temperature; 25° C.
Carrier gas; Nitrogen
Carrier gas flow rate; 200 mL·min⁻¹ (Total)

Typically, 20-50 mg of the test substance was mounted in a gauze basket and transferred to the sample port. Excess static electricity was removed throughout sample handling and sample mounting using a ²¹⁰Po static eliminator. Instrument performance checks were completed using traceable samples of sodium chloride and lithium chloride. For selected samples, the cycle was repeated using the same sample to assess any change in physical form induced on exposure to elevated relative humidity.

### ¹H Nuclear Magnetic Resonance (NMR) Spectroscopy

An assessment of counter-ion stoichiometry was performed using 1H nuclear magnetic resonance (NMR) spectroscopy.

The samples were stored at ambient laboratory temperature (18-22° C.) until required for analysis. For NMR analyses, the materials were dissolved in 0.6 mL deuterated dimethylsulfoxide (DMSO-d6) and subsequently transferred to a high precision 5 mm OD NMR tube. In each case, the drug substance samples readily dissolved without the necessity for heating. Analyses were initiated within 60 minutes of dissolving the material in deuterated solvent, following optimization and stabilization of the magnet.

All NMR experiments were performed using a Bruker Avance III HD spectrometer equipped with an 11.7 Tesla magnet. Spectra were acquired using a 5 mm BBO ¹H multinuclear z-gradient broadband normal geometry probe. Sample temperatures were controlled throughout the analyses, with appropriate time allowed for establishment of thermal equilibrium.

¹H NMR spectra were acquired with a direct pulse-acquire sequence and the following parameters were employed for each of the materials analyzed:

| Observation frequency (¹H) | 500.13 | MHz |
|---|---|---|
| Complex data points | 65536 (with | |
| | no zero-filling) | |
| Acquisition time | 4.7 | s |
| Spectral width | 7.0 | kHz |
| Relaxation delay | 1.0 | s |
| Pulse width | π/6 (3.46 μs) | |
| Number of dummy transients | 2 | |
| Number of transients | 16 | |
| Temperature | 298 | K |

Chemical shifts were referenced with respect to tetramethylsilane (TMS), indirectly, using the residual proton signal of DMSO-d₆. An internal reference was not employed to circumvent the potential for induced degradation. An exponential multiplication convolution of 0.3 Hz was applied to the free induction decay prior to Fourier transformation.

### III. Results

Samples were analyzed by differential scanning calorimetry (DSC), X-ray powder diffraction (XRPD), and dynamic vapor sorption (DVS). In addition, the appearance and visual properties of each sample were assessed, in addition to propensity for crystallization. Some of the principal target properties obtained from each analytical assessment are listed in Table 9.

A summary of the data obtained from the analyses of the individual salt forms of Examples 1-9 are presented in Table 10, with advantageous and detrimental properties summarized using the following nomenclature: “+/+/+” indicates most favorable; “+/+” indicates more favorable; “+” indicates favorable; “−” indicates unfavorable; “−/−” indicates more unfavorable; “−/−/−” indicates most unfavorable.

TABLE 9
Target properties of salt forms

| | |
|---|---|
| Analytical assessment | Target observation/property |
| Differential scanning | High melting point |
| calorimetry (DSC) | High enthalpy of fusion |
| | No low melting events |
| X-ray powder | Indicative of one physical form |
| diffraction (XRPD) | High degree of crystallinity |
| Dynamic vapor | Little change in isotherm over ambient |
| sorption (DVS) | relative humidities |
| | Little/no hysteresis |
| | No physical conversion on exposure to |
| | elevated relative humidities |
| | No physical conversion on exposure to 0% |
| | RH or >80% RH |
| Appearance | Free flowing (white/off-white) powder |
| | Absence of cohesion or adhesion to surfaces |
| | Regular morphology |
| Crystallization | Readily affords a crystalline solid on |
| | crystallization |
| | Avoid proceeding via oil |
| | Reasonable yield |
| | Favorable volume factors |

TABLE 10
Summary overview of salt properties of Examples 1-9

| | Ex. 1 | Ex. 2 | Ex. 3 | Ex. 4 | Ex. 5 | Ex. 6 | Ex. 7 | Ex. 8/9^{a} |
|---|---|---|---|---|---|---|---|---|
| | (I-1a) | (I-1b) | (I-1c) | (I-1d) | (I-1e) | (I-1f) | (I-1g) | (I-1h/I-1i) |
| Crystallization propensity | +/+/+ | +/+/+ | + | − | + | − | − | − |
| Physical properties | +/+/+ | +/+/+ | +/+/+ | +/+/+ | +/+ | +/+ | −/− | −/− |
| (crystallinity, melt, enthalpy, polymorph tendency) | | | | | | | | |
| Hygroscopicity | +/+/+ | +/+/+ | +/+/+ | +/+/+ | − | −/−/− | −/−/− | −/−/− |
| Appearance | +/+/+ | +/+/+ | +/+/+ | +/+/+ | +/+ | +/+/+ | − | − |
| Counter-ion physiological acceptability | +/+/+ | +/+/+ | + | +/+/+ | + | + | + | +/+/+ |
| Salt suitability | +/+/+ | +/+/+ | +/+ | + | − | −/−/− | −/−/− | −/−/− |

^{a}Combined results from both crystalline polymorphs

X-ray powder diffraction (XRPD)

The X-ray powder diffractograms of Example 6 (I-1f; glycolate), Example 9 (I-1i; hemi-fumarate), and Example 1 (I-1a; fumarate) are presented in FIG. 3. The X-ray powder diffractograms of Example 2 (I-1b; benzoate), Example 3 (I-1c; salicylate), and Example 7 (I-1g; hemi-oxalate) are presented in FIG. 4. The X-ray powder diffractograms of Example 5 (I-1e; oxalate), Example 4 (I-1d; succinate), and Example 8 (I-1h; hemi-fumarate) are presented in FIG. 5. Characteristic X-ray diffraction peaks (2θ±0.2°) for Examples 1-8 are tabulated in Table 11. In each case, the diffractograms comprise discrete and sharp Bragg diffractions consistent with crystalline materials. The diffraction patterns are different for each salt form, reflecting the differing crystal lattice parameters. However, it is noted that the two samples of the hemi-fumarate (Examples 8 and 9) showed slightly different diffraction patterns, indicating polymorphic tendency for this salt form. There are some common diffractions for the latter salt, confirming that one or both samples comprise a physical mixture. With the exception of the hemi-fumarate, subjectively, the samples provided comprise one physical form. Some slight broadening of the Bragg diffractions is noted for the hemi-oxalate (Example 7; I-1g); the effect is very small but indicates some disorder or reduced crystallinity.

The X-ray powder diffraction studies showed that all salt forms included as part of the present study were crystalline and suitable for salt form assessments. It is noted that the hemi-fumarate (Examples 8 and 9) exhibited some polymorphism tendency, which is not desirable. Whilst of little consequence, it is also noted that the hemi-oxalate (Example 7; I-1g) showed some minor broadening of the diffractions suggesting slight disorder in the crystallinity or presence of low quantities of amorphous material. Both observations associated with the hemi-fumarate and hemi-oxalate are not completely prohibitive, but render them less favorable for pharmaceutical development purposes when compared with the other salt forms.

TABLE 11
Characteristic XRD peaks (2θ ± 0.2°) for Examples 1-8

| | | | | | | | |
|---|---|---|---|---|---|---|---|
| Example 1 | Example 2 | Example 3 | Example 4 | Example 5 | Example 6 | Example 7 | Example 8 |
| (I-1a) | (I-1b) | (I-1c) | (I-1d) | (I-1e) | (I-1f) | (I-1g) | (I-1h) |

2θ ± 0.2°

| | | | | | | | |
|---|---|---|---|---|---|---|---|
| 7.8 | 9.6 | 9.6 | 9.8 | 11.3 | 8.2 | 8.7 | 8.1 |
| 10.3 | 11.1 | 10.5 | 11.7 | 12.3 | 12.2 | 11.5 | 11.3 |
| 10.9 | 12.6 | 14.9 | 14.3 | 15.6 | 12.9 | 13.6 | 12.2 |
| 13.6 | 13.5 | 17.1 | 14.7 | 17.7 | 15.8 | 14.2 | 13.3 |
| 15.8 | 15.8 | 18.1 | 17.0 | 19.5 | 16.3 | 15.2 | 14.2 |
| 16.1 | 16.1 | 19.1 | 17.4 | 20.0 | 17.8 | 17.4 | 16.2 |
| 17.0 | 17.1 | 20.1 | 19.6 | 20.8 | 19.2 | 17.6 | 17.6 |
| 18.4 | 17.9 | 20.7 | 20.6 | 21.4 | 20.1 | 18.0 | 18.3 |
| 19.7 | 19.8 | 21.0 | 22.3 | 22.3 | 21.7 | 19.3 | 18.6 |
| 19.9 | 20.1 | 21.3 | 22.6 | 22.7 | 23.6 | 19.6 | 19.5 |
| 20.6 | 20.8 | 24.6 | 22.9 | 24.8 | 24.4 | 20.1 | 19.8 |
| 21.3 | 21.2 | 25.6 | 23.1 | 25.7 | 24.6 | 20.6 | 20.0 |
| 21.7 | 22.7 | 28.5 | 23.4 | 26.7 | 24.9 | 21.9 | 20.2 |
| 22.5 | 23.8 | 28.8 | 24.9 | 27.9 | 26.0 | 22.1 | 20.9 |
| 23.9 | 24.6 | 29.4 | 25.2 | 28.7 | 26.6 | 22.9 | 21.4 |
| 24.1 | 26.9 | 30.3 | 26.3 | 29.5 | 27.8 | 23.2 | 21.9 |
| 25.1 | 29.2 | 31.3 | 26.8 | 31.4 | 29.6 | 23.5 | 22.3 |
| 26.2 | 32.3 | 32.1 | 27.3 | 33.0 | 30.2 | 24.5 | 22.7 |
| 33.6 | 35.1 | 33.5 | 27.7 | 35.4 | 32.0 | 25.0 | 22.9 |
| 34.9 | 36.1 | 34.4 | 28.8 | 36.5 | 32.3 | 25.5 | 23.8 |
| | | | 29.1 | 38.6 | 33.0 | 26.1 | 24.5 |
| | | | 30.9 | | 33.9 | 26.4 | 25.0 |
| | | | 31.5 | | 34.6 | 27.1 | 25.2 |
| | | | 33.8 | | | 28.4 | 26.1 |
| | | | 34.5 | | | 28.7 | 26.4 |
| | | | 36.5 | | | 29.8 | 26.9 |
| | | | 39.2 | | | 30.4 | 28.4 |
| | | | | | | 30.7 | 28.8 |
| | | | | | | 31.4 | 29.5 |
| | | | | | | 31.8 | 29.8 |
| | | | | | | 33.4 | 30.9 |
| | | | | | | 33.9 | 32.7 |

The X-ray powder diffractogram of Example 26 (I-8a; fumarate), in comparison to Example 1 (I-1a; fumarate), is presented in FIG. 6. The X-ray powder diffractogram of Example 27 (I-8b; benzoate), in comparison to Example 2 (I-1b; benzoate), is presented in FIG. 7. The X-ray powder diffractogram of Example 28 (I-8c; salicylate), in comparison to Example 3 (I-1c; salicylate), is presented in FIG. 8. Characteristic X-ray diffraction peaks (2θ±0.2°) for Examples 26-28 are tabulated in Table 12. As can be seen, the X-ray powder diffractograms of the deuterated (DMT d-10) salts are the same as, or nearly identical to, their respective protonated analogs. Thus, it was concluded that the physical forms of the deuterated salts are the same as, or nearly identical to, the protonated salts. Accordingly, the advantageous properties such as those identified in Tables 9 and 10 (e.g., crystallization propensity, physical properties, hygroscopicity, appearance, physiological acceptability, and overall salt suitability) can be concluded to extend to the deuterated salt forms.

TABLE 12
Characteristic XRD peaks (2θ ± 0.2°) for Examples 26-28

| | | |
|---|---|---|
| Example 26 (1-8a) | Example 27 (1-8b) | Example 28 (1-8c) |

2θ ± 0.2°

| | | | | |
|---|---|---|---|---|
| 7.8 | 29.3 | 9.6 | 9.6 | 29.4 |
| 10.3 | 29.6 | 11.1 | 10.5 | 29.7 |
| 10.9 | 29.9 | 12.7 | 11.4 | 30.3 |
| 12.5 | 30.6 | 13.5 | 12.3 | 31.0 |
| 13.6 | 31.0 | 15.8 | 13.4 | 31.3 |
| 14.6 | 31.3 | 16.1 | 14.2 | 32.1 |
| 15.2 | 32.4 | 17.2 | 14.9 | 32.7 |
| 15.5 | 32.9 | 17.9 | 15.6 | 33.1 |
| 15.8 | 33.3 | 19.8 | 16.1 | 33.5 |
| 16.1 | 33.6 | 20.1 | 17.1 | 34.4 |
| 16.6 | 34.3 | 20.8 | 18.1 | 35.0 |
| 17.0 | 34.9 | 21.2 | 18.7 | |
| 18.4 | 35.7 | 22.8 | 19.1 | |
| 19.0 | 36.1 | 23.8 | 20.1 | |
| 19.7 | 37.4 | 24.3 | 20.8 | |
| 19.9 | 38.0 | 24.6 | 21.1 | |
| 20.6 | 38.5 | 25.1 | 21.3 | |
| 21.3 | | 25.3 | 22.2 | |
| 21.8 | | 25.5 | 22.6 | |
| 22.5 | | 26.9 | 23.7 | |
| 23.3 | | 28.3 | 24.6 | |
| 23.8 | | 28.9 | 25.2 | |
| 24.1 | | 29.3 | 25.6 | |
| 25.1 | | 31.4 | 26.1 | |
| 26.2 | | 31.6 | 26.4 | |
| 26.8 | | 32.0 | 27.4 | |
| 27.3 | | 32.3 | 27.5 | |
| 27.9 | | 32.8 | 27.8 | |
| 28.3 | | 35.1 | 28.5 | |
| 28.9 | | 36.1 | 28.8 | |

### Differential Scanning Calorimetry (DSC)

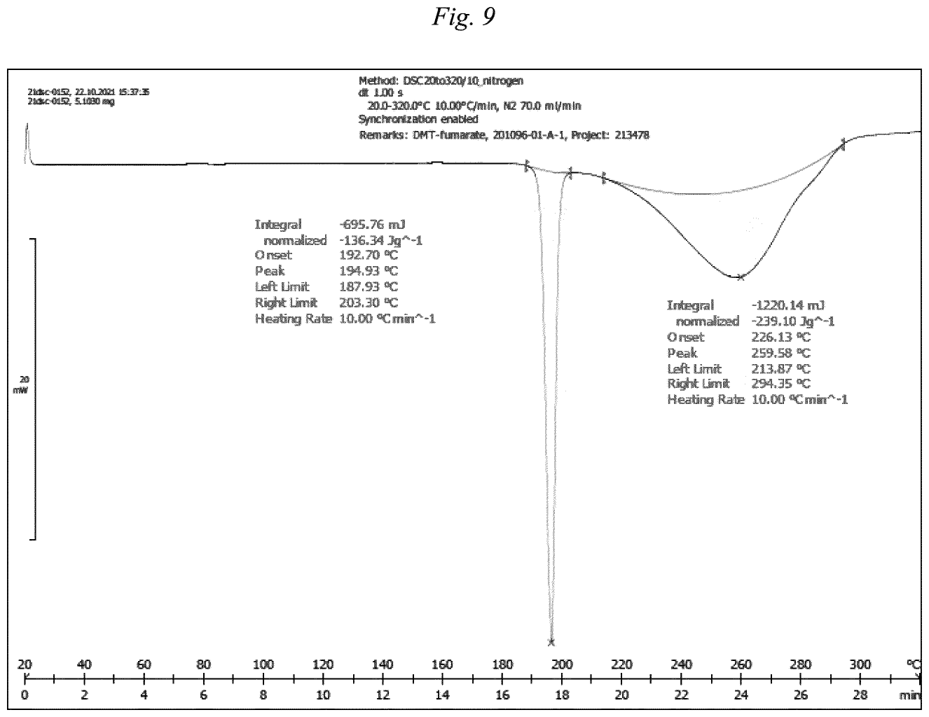

The DSC curve of Example 1 (I-1a; fumarate) is shown in FIG. 9, and it is evident that the salt has a high melt onset (192.7° C.) and high enthalpy of fusion (136.3 J·g⁻¹), which are favorable for pharmaceutical development. Equally, no events were observed prior to the melt endotherm, indicating the absence of multiple physical forms in the sample, and also no conversion of physical forms prior to the melt. Post-melt events are ascribed to decomposition.

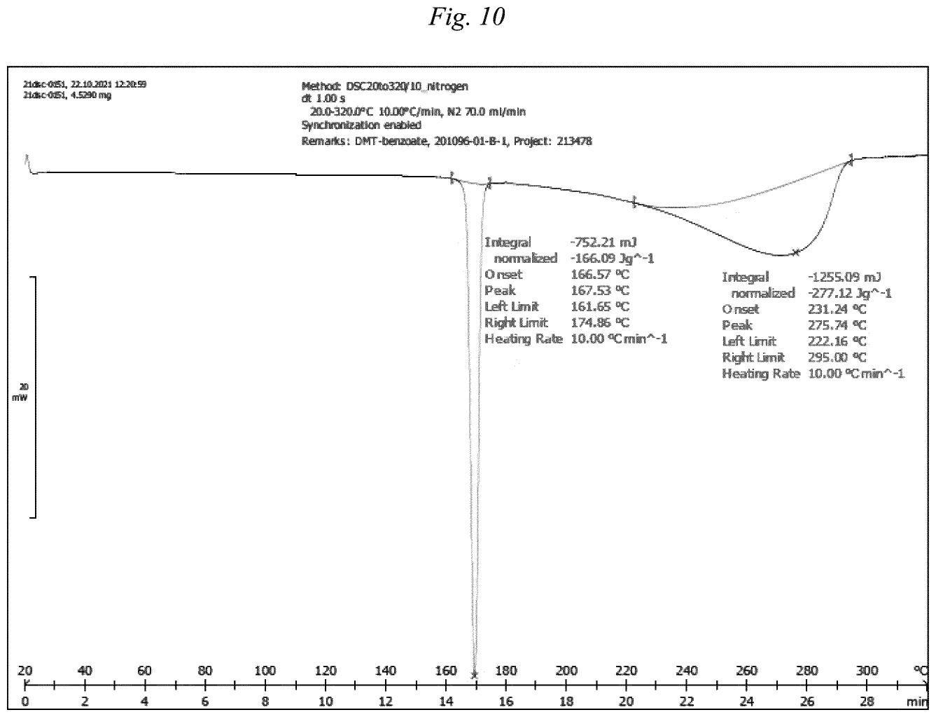

The DSC curve of Example 2 (I-1b; benzoate) is shown in FIG. 10 and, similarly to the fumarate, it is evident that the salt has a high melt onset (166.6° C.) and high enthalpy of fusion (166.1 J·g⁻¹). Equally, no events were observed prior to the melt endotherm, indicating the absence of multiple physical forms in the sample, and also no conversion of physical forms prior to the melt. Post-melt events are ascribed to decomposition.

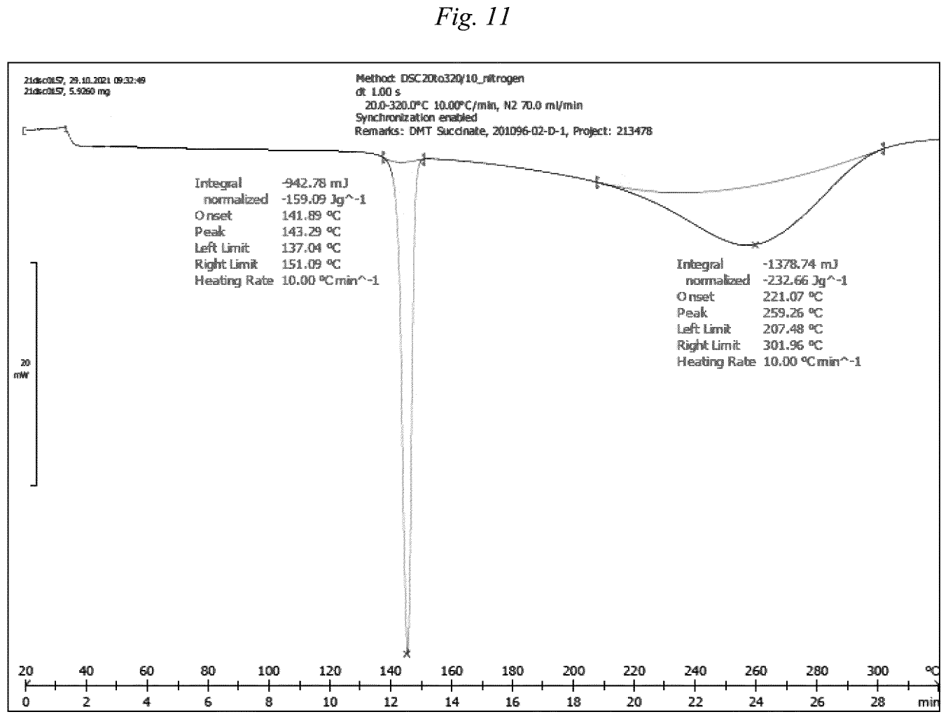

The DSC curve of Example 4 (I-1d; succinate) is shown in FIG. 11 and, similarly to the fumarate and benzoate, it is evident that the salt has a high melt onset (141.9° C.) and high enthalpy of fusion (159.1 J·g⁻¹). Equally, no events were observed prior to the melt endotherm, indicating the absence of multiple physical forms in the sample, and also no conversion of physical forms prior to the melt. Post-melt events are ascribed to decomposition.

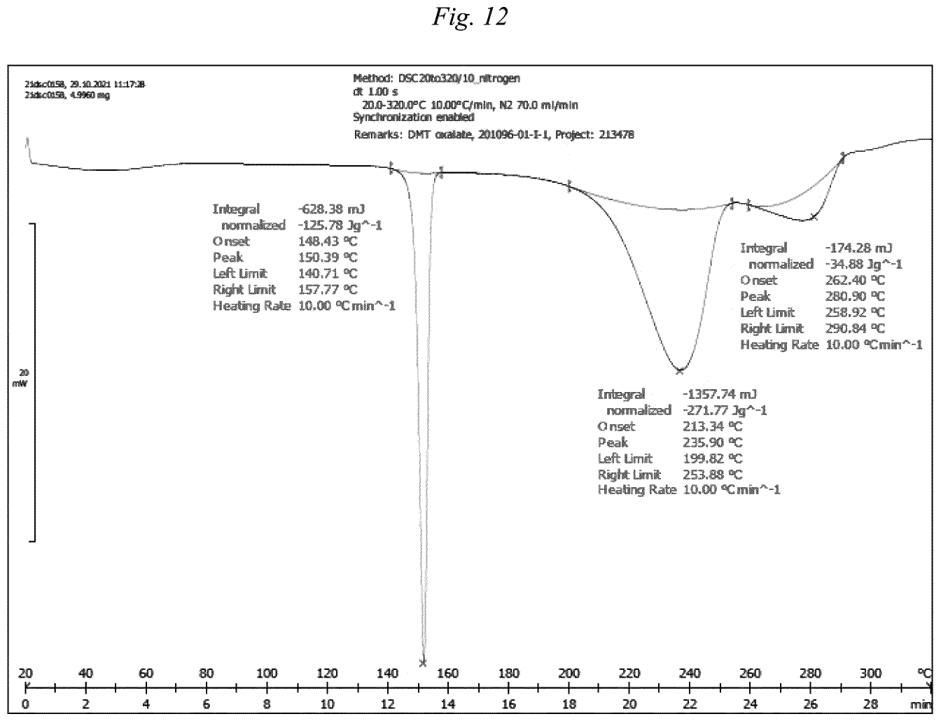

The DSC curve of Example 5 (I-1e; oxalate) is shown in FIG. 12 and, similarly to the preceding salts, it is evident that the salt has a high melt onset (148.4° C.) and high enthalpy of fusion (125.8 J·g⁻¹). Equally, no significant events were observed prior to the melt endotherm, indicating the absence of multiple physical forms in the sample, and also no conversion of physical forms prior to the melt; the broad low intensity endotherm between 20° C. and 80° C. is due to loss of water/solvent. Post-melt events are ascribed to decomposition.

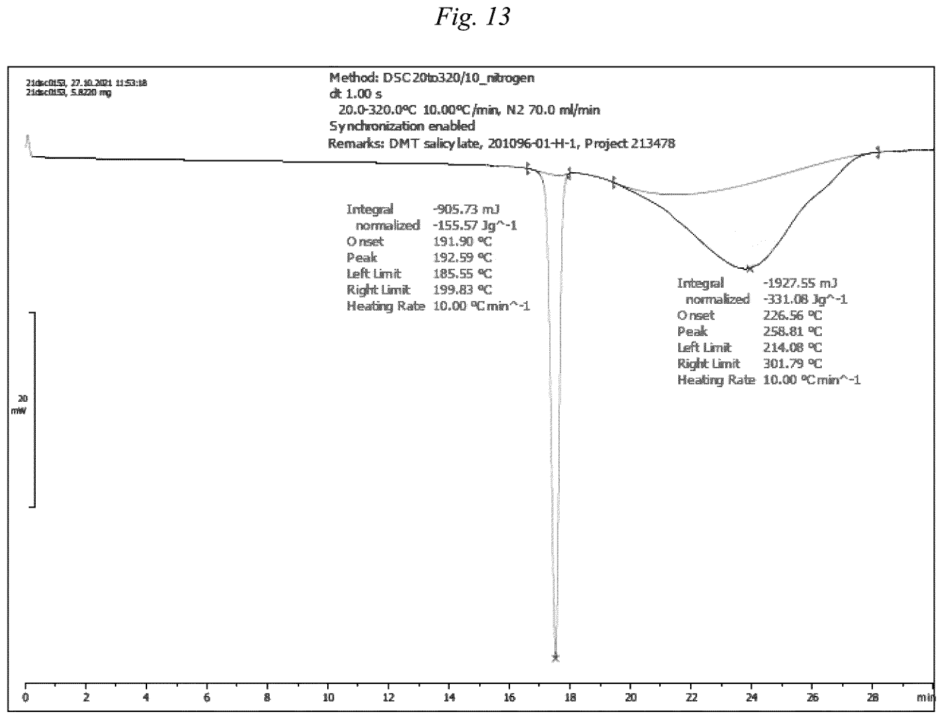

The DSC curve of Example 3 (I-1c; salicylate) is shown in FIG. 13 and, similarly to the preceding salts, it is evident that the salt has a high melt onset (191.9° C.) and high enthalpy of fusion (155.6 J·g⁻¹). Equally, no events were observed prior to the melt endotherm, indicating the absence of multiple physical forms in the sample, and also no conversion of physical forms prior to the melt. Post-melt events are ascribed to decomposition.

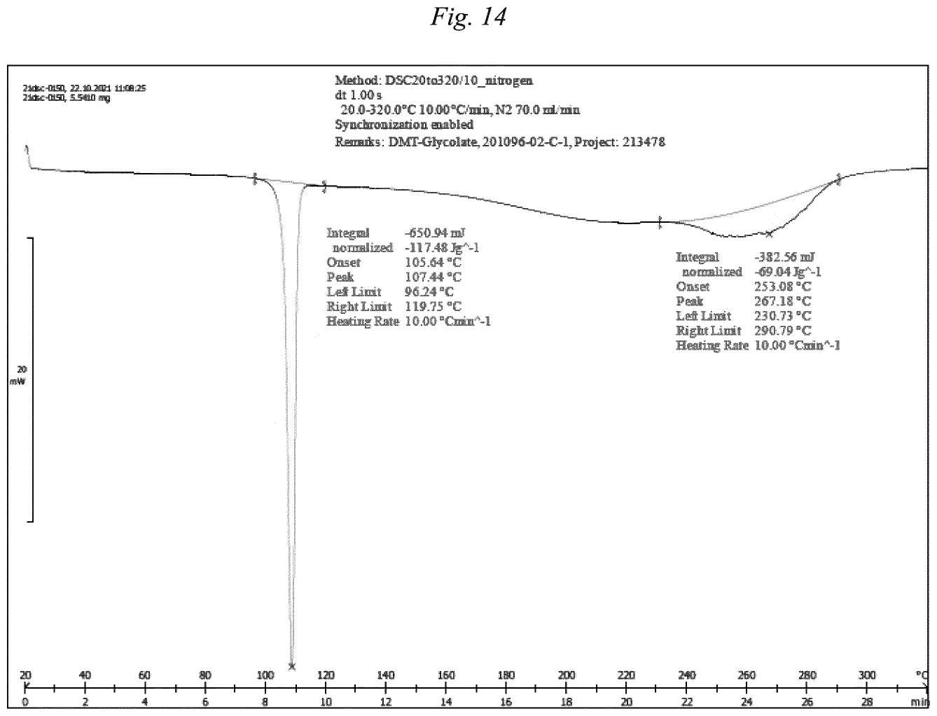

The DSC curve of Example 6 (I-1f; glycolate) is shown in FIG. 14. The melt onset (105.6° C.) and enthalpy of fusion (117.5 J·g⁻¹) are somewhat lower than the salts described previously, but are still acceptable for pharmaceutical development purposes. The excellent physical characteristics of the other salts render the glycolate slightly less favorable in this respect. No events were observed prior to the melt endotherm, indicating the absence of multiple physical forms in the sample, and also no conversion of physical forms prior to the melt. Post-melt events are ascribed to decomposition.

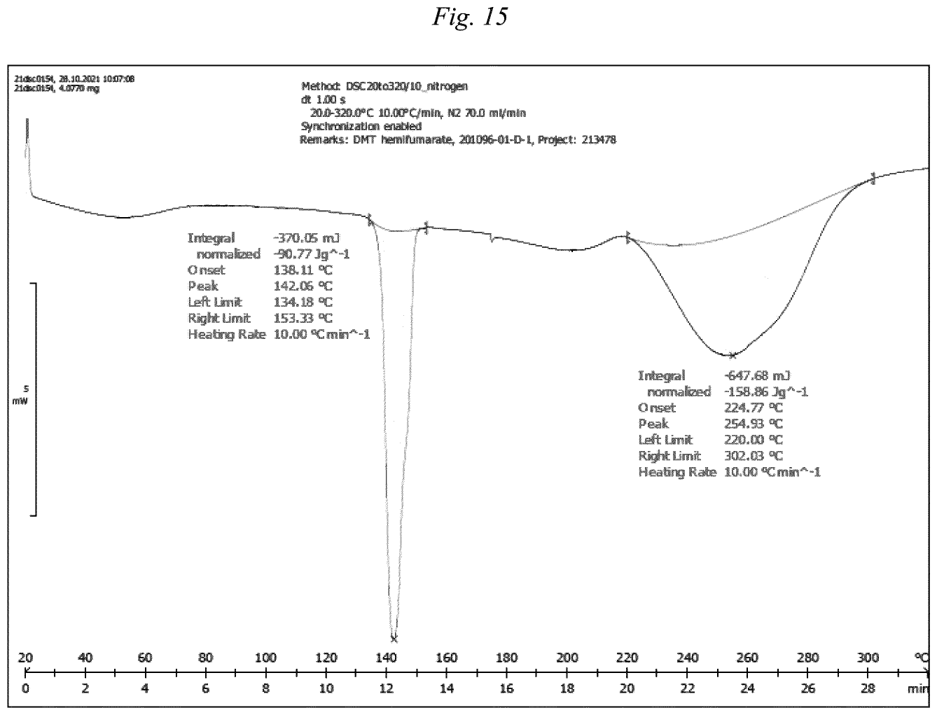

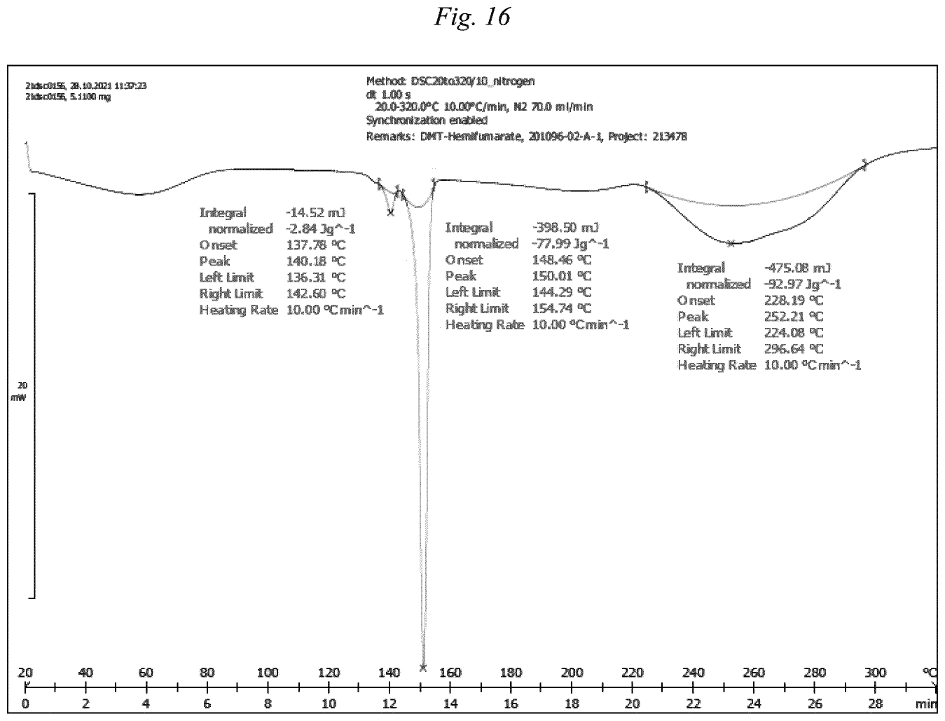

DSC curves of Example 9 (I-1i; hemi-fumarate) and Example 8 (I-1h; hemi-fumarate) are shown in FIGS. 15 and 16, respectively. The melt onsets were quite high (typically >138° C.) but the enthalpy of fusion is a little lower than other salts (ca. 90 J·g¹). In Example 9, the endotherm is slightly broadened, consistent with the presence of physical or chemical impurities and for the other batch a small endothermic event is observed immediately prior to the main melt. Both DSC curves confirm the tendency for multiple physical forms, consistent with the X-ray powder diffraction data presented previously. The presence of multiple forms or a tendency to exhibit multiple forms is not completely prohibitive but, where alternatives exist, such salt forms are not as advantageous as others. Post-melt events are ascribed to decomposition.

In summary, with the exception of the hemi-fumarate, all tested salt forms exhibited satisfactory melt onset temperatures and enthalpies of fusion, with no prohibitive polymorph tendencies. The hemi-fumarate clearly shows the presence of different physical forms. Whilst none of the salt forms show characteristics that could not be addressed for pharmaceutical development purposes, there are clearly preferred candidates solely considering the DSC data, namely fumarate, benzoate, salicylate and succinate.

### Dynamic Vapor Sorption (DVS)

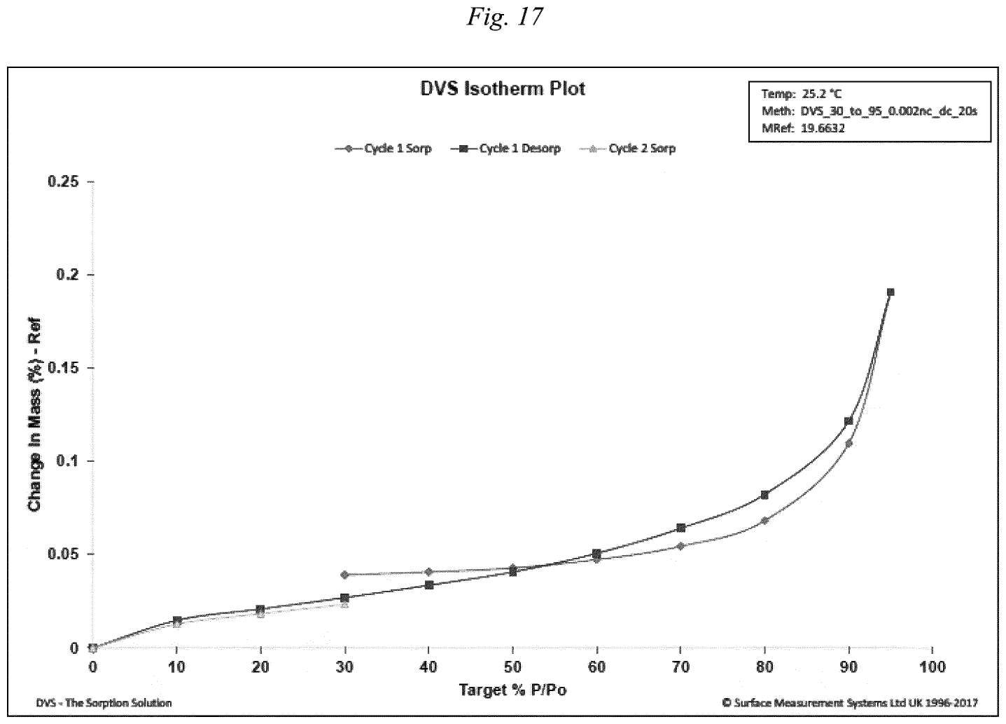

The DVS isotherm plot of Example 1 (I-1a; fumarate) is shown in FIG. 17. There is no significant hygroscopicity and the acquisition of water was very low, even on exposure to elevated relative humidities of >95% RH (<0.2% w/w). The change in mass was low over the typical range of ambient relative humidities, and there is no evidence of physical form conversion throughout the cycle. This isotherm plot represents advantageous behavior for pharmaceutical development purposes.

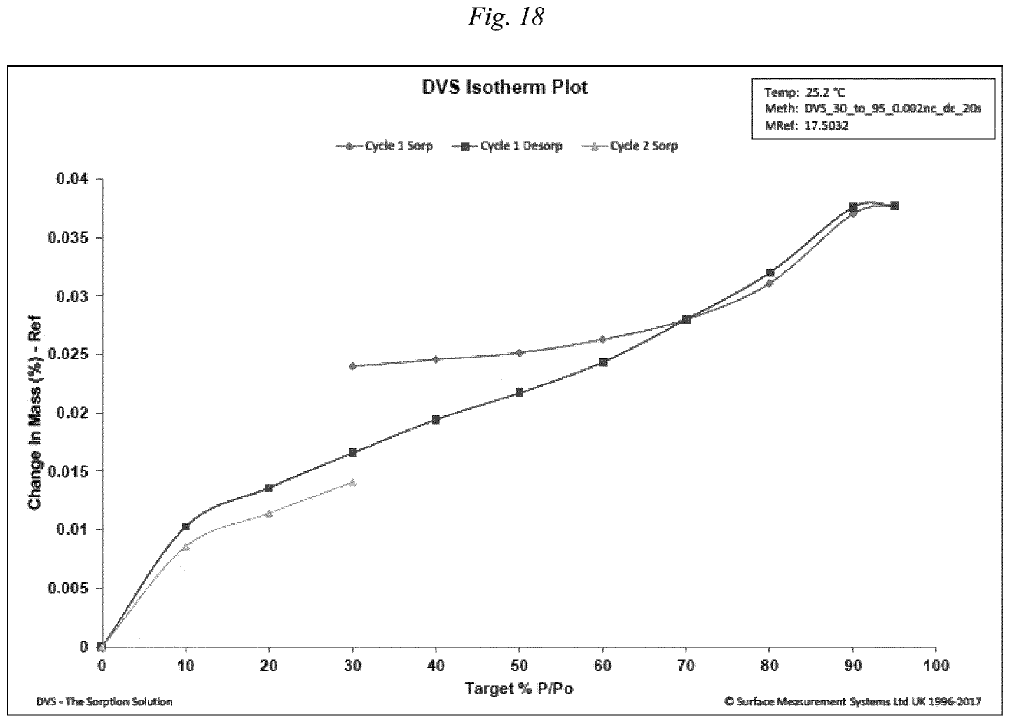

The DVS isotherm plot of Example 2 (I-1b; benzoate) is shown in FIG. 18. As for the fumarate salt, there is no significant hygroscopicity and the acquisition of water was very low, even on exposure to elevated relative humidities of >95% RH (<0.05% w/w). The change in mass was low over the typical range of ambient relative humidities, and there is no evidence of physical form conversion throughout the cycle. This isotherm plot represents advantageous behavior for pharmaceutical development purposes.

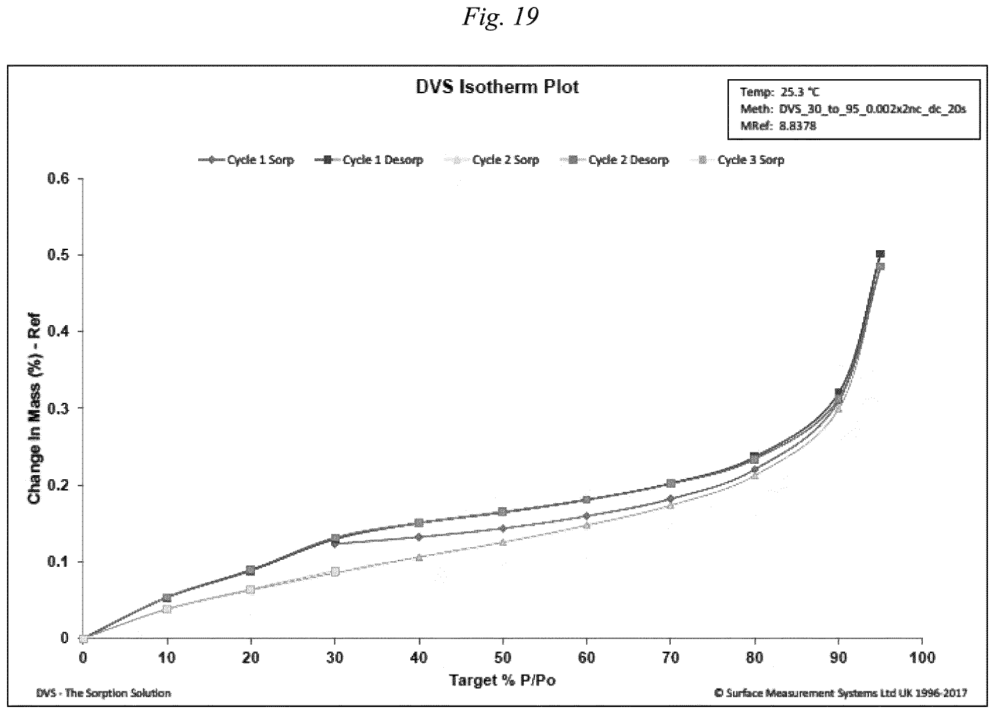

The DVS isotherm plot of Example 4 (I-1d; succinate) is shown in FIG. 19. Akin to the fumarate and benzoate salts, there is no significant hygroscopicity and the acquisition of water was very low, even on exposure to elevated relative humidities of >95% RH (<0.5% w/w). Whilst there was some increased uptake of water on exposure to 95% RH, the level is quite acceptable and the profile between 30% RH and 80% RH is quite flat, such that no significant change in mass will occur over typical ambient relative humidities. There is no evidence of physical form conversion throughout the cycle. This isotherm plot represents an acceptable behavior for pharmaceutical development purposes.

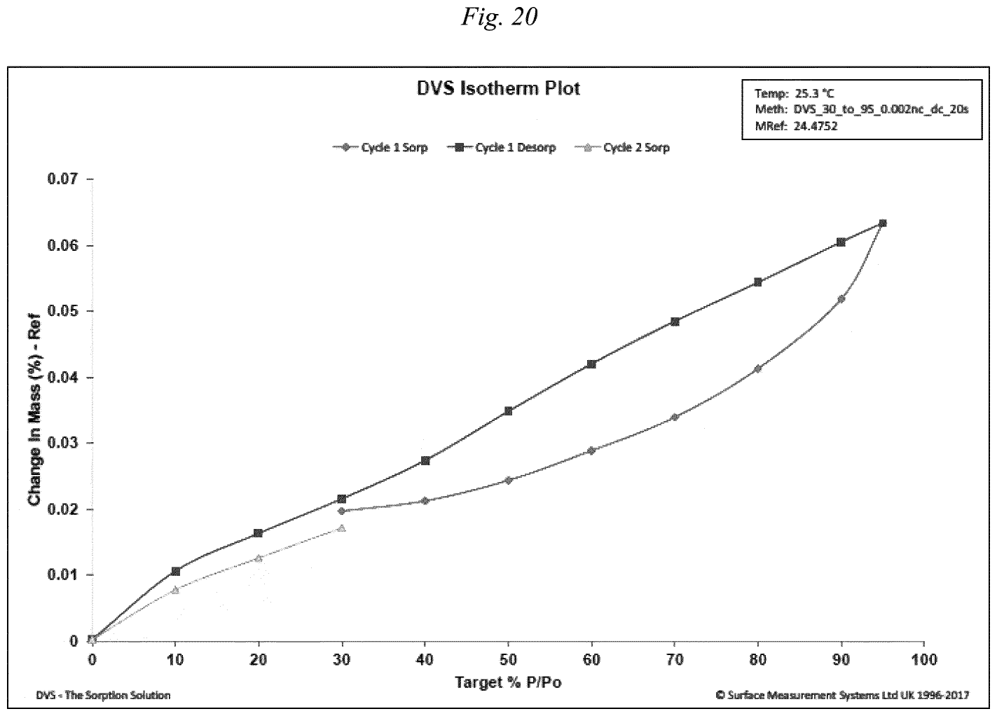

The DVS isotherm plot of Example 3 (I-1c; salicylate) is shown in FIG. 20. There is no significant hygroscopicity and the acquisition of water was very low, even on exposure to elevated relative humidities of >95% RH (<0.06% w/w). The change in mass was low over the typical range of ambient relative humidities, and there is no evidence of physical form conversion throughout the cycle. This isotherm plot represents advantageous behavior for pharmaceutical development purposes.

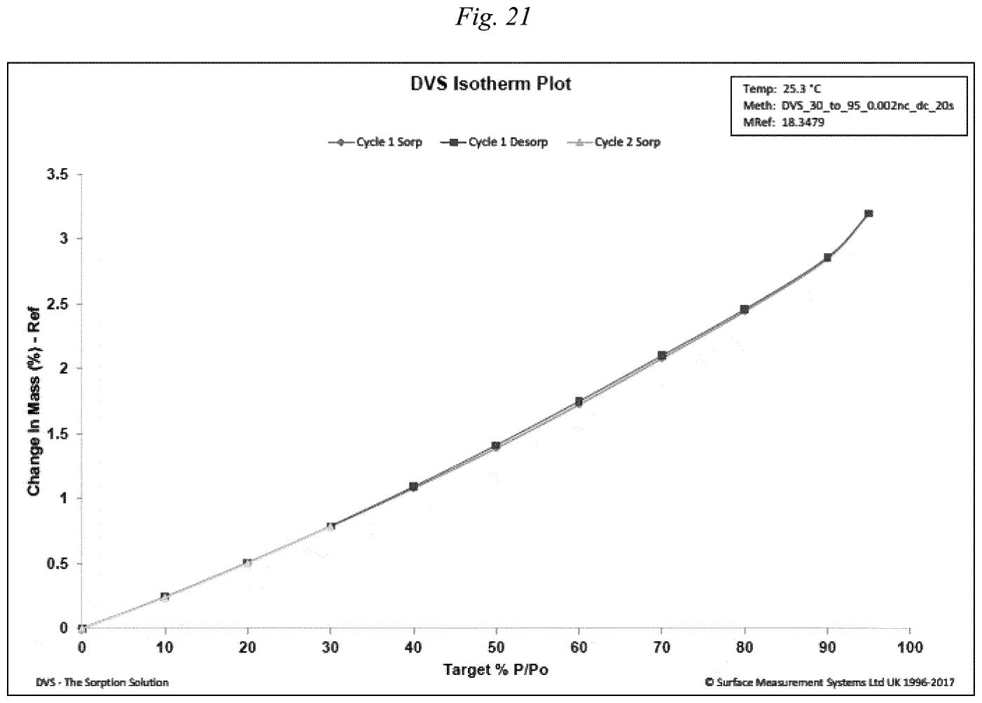

The DVS isotherm plot of Example 5 (I-1e; oxalate) is shown in FIG. 21. In this case, there was a water uptake of approximately 3% w/w on exposure to 95% RH, and there is no recognizable plateau at any point between 0% RH and 95% RH. This profile is not preferred and would likely give rise to some challenges from an analytical perspective. Generally, such behavior is also accompanied with crystallization challenges.

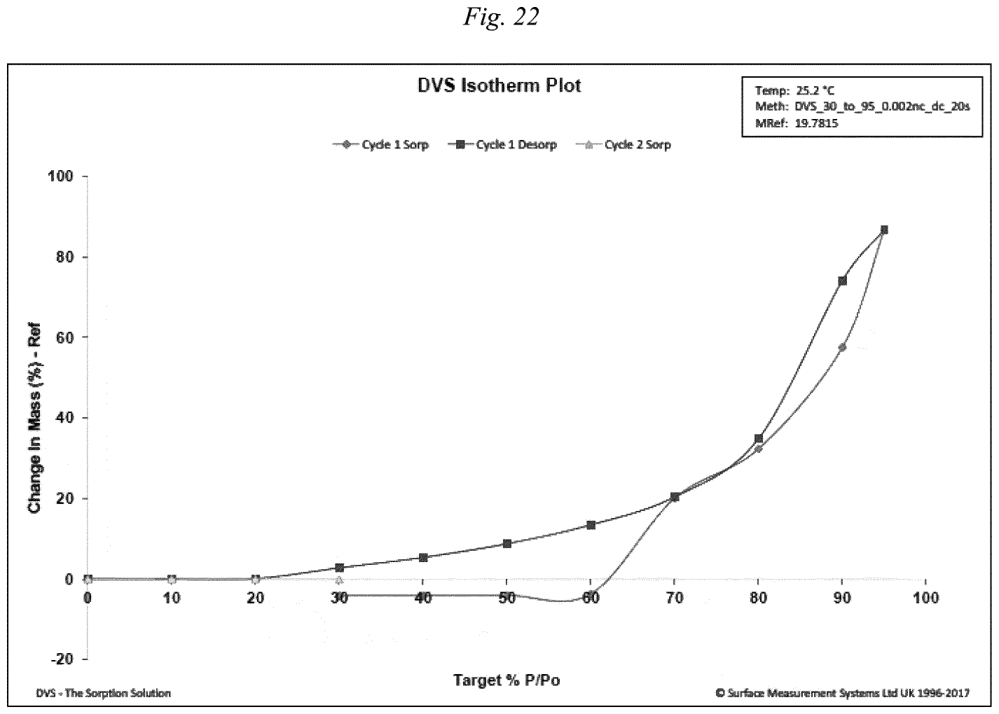

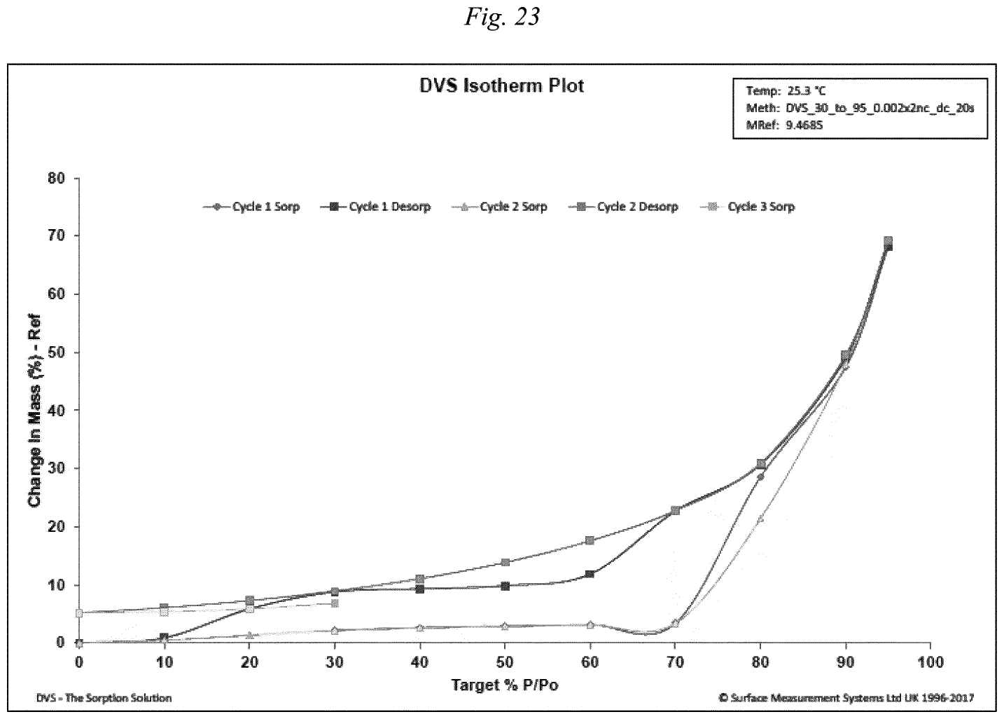

The DVS isotherm plot of Example 6 (I-1f; glycolate) and Example 8 (I-1h; hemi-fumarate) are shown in FIGS. 22 and 23, respectively. These salt forms effectively deliquesce at elevated relative humidities, which is not preferred for pharmaceutical development.

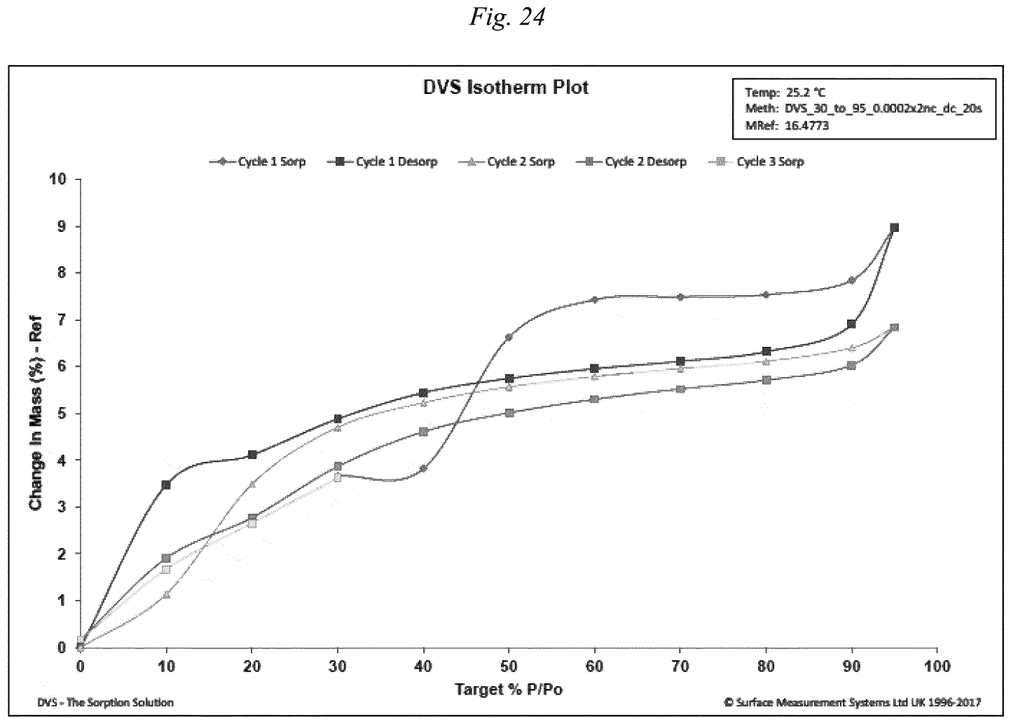

The DVS isotherm plot of Example 7 (I-1g; hemi-oxalate) is shown in FIG. 24. The equilibrium water content at typical ambient relative humidities is >3% and quite variable as a function of relative humidity. Indeed, the water uptake at relative humidities exceeding 80% was excessive and, based on the reproducibility of the two cycles, a phase change was induced on exposure to elevated relative humidity. This salt form may not be suitable for pharmaceutical development in the absence of additional work to investigate alternative physical forms.

In summary, the vapor sorption behaviors for the salt forms assessed herein were quite varied and differentiating. The profiles for the fumarate, benzoate, succinate and salicylate are all highly favorable, with no deliquescence and low uptake of water over typical ambient relative humidities.

### ¹H Nuclear Magnetic Resonance (NMR) Spectroscopy

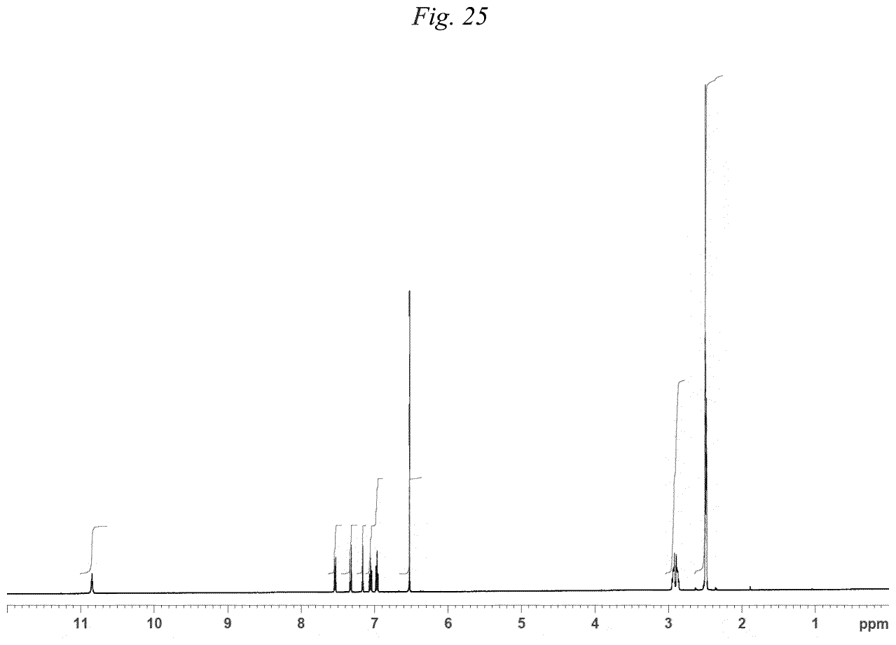

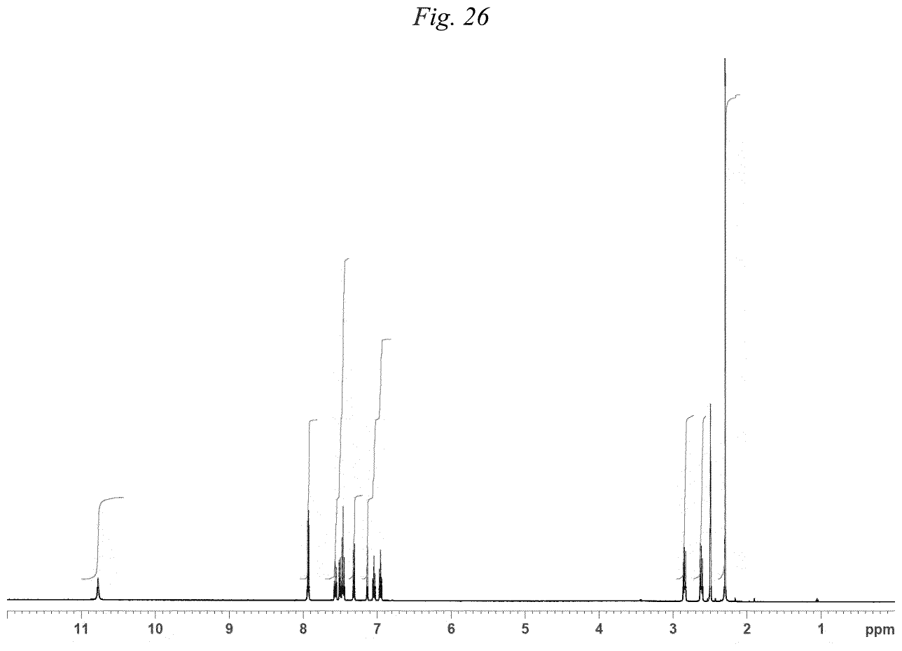

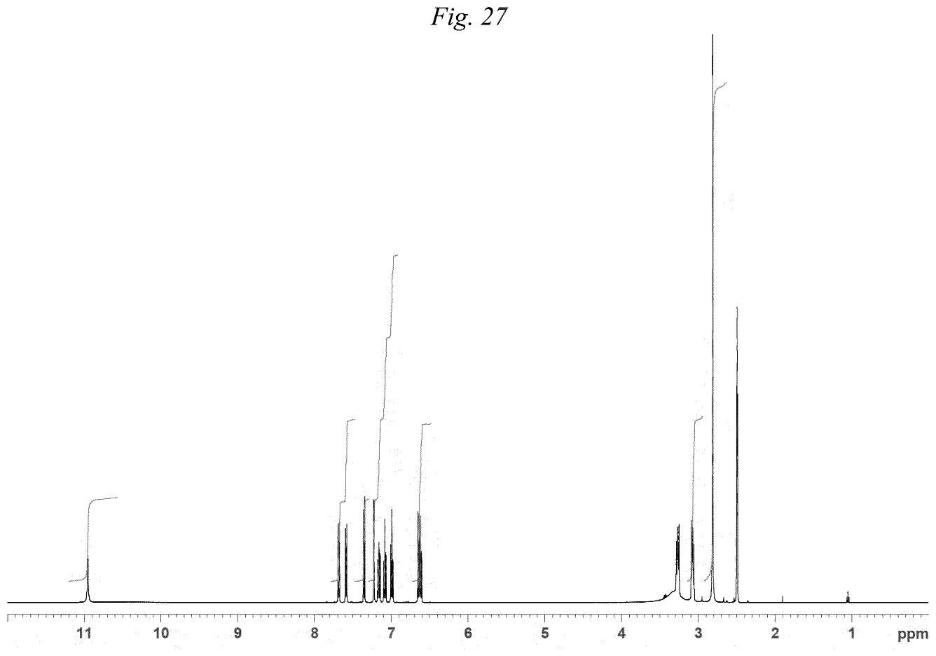

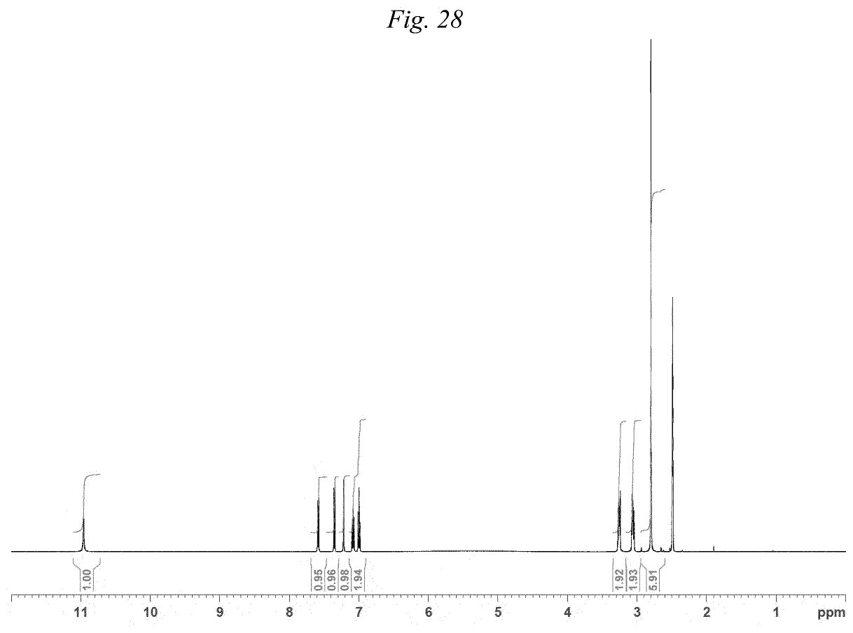

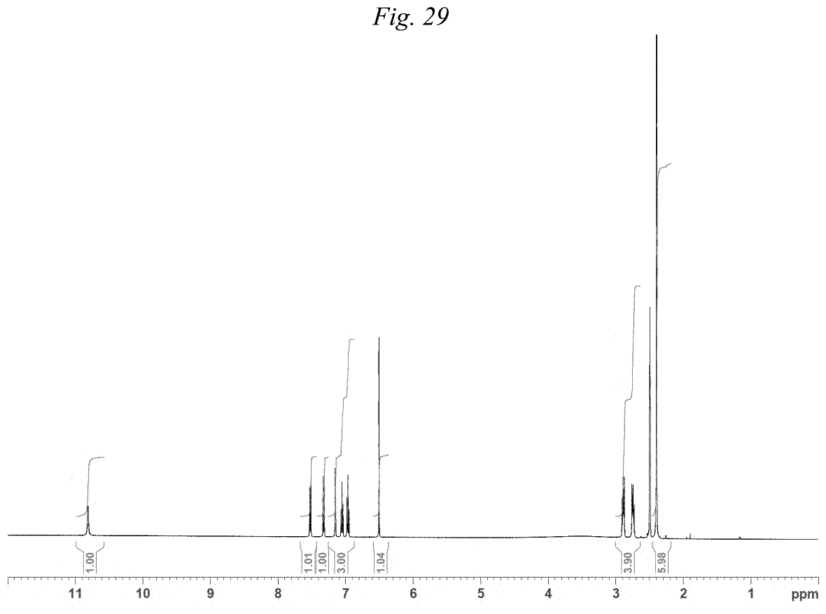

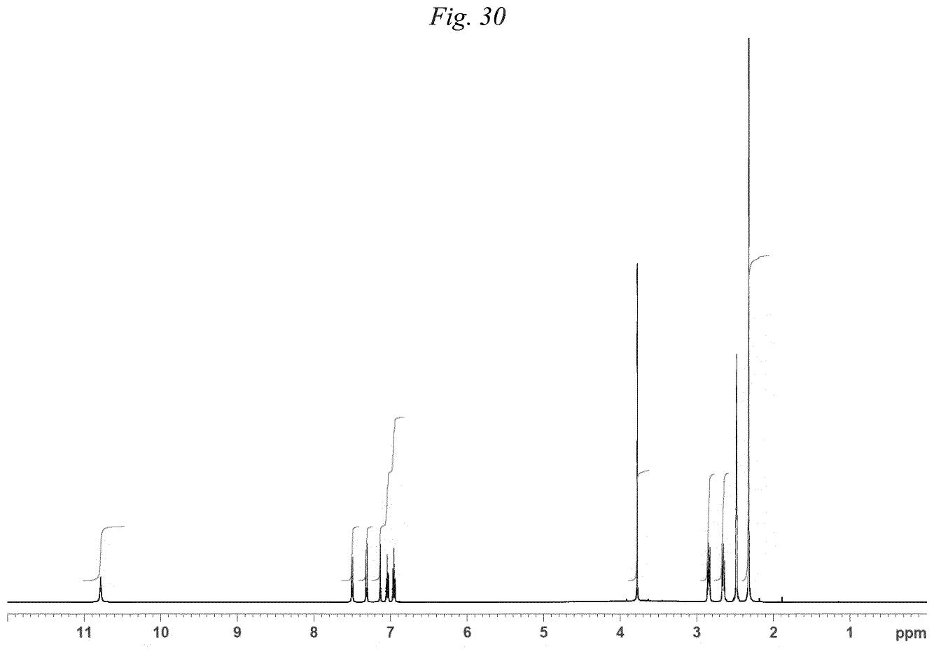

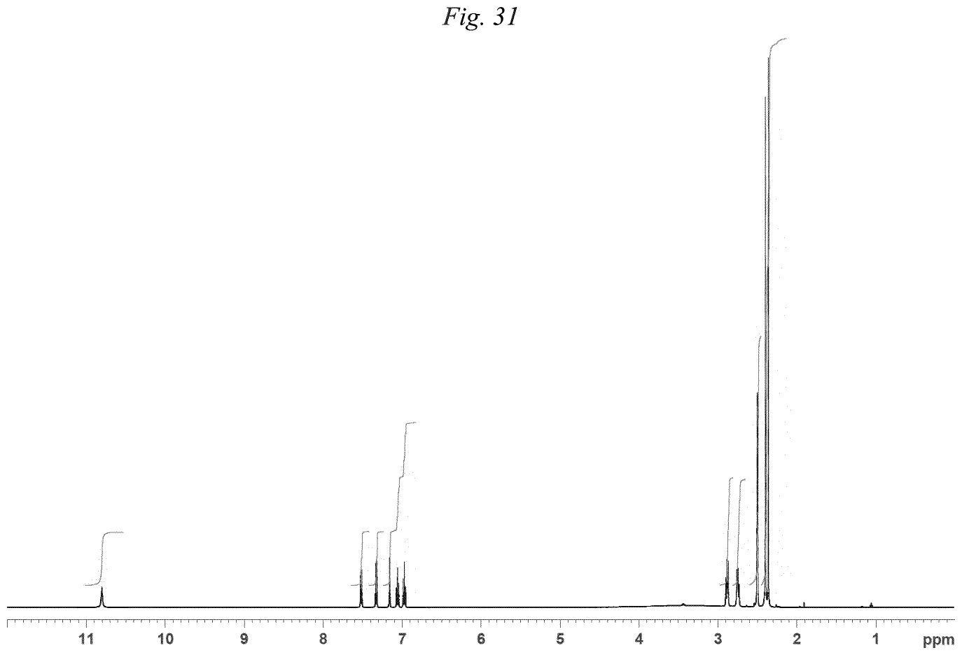

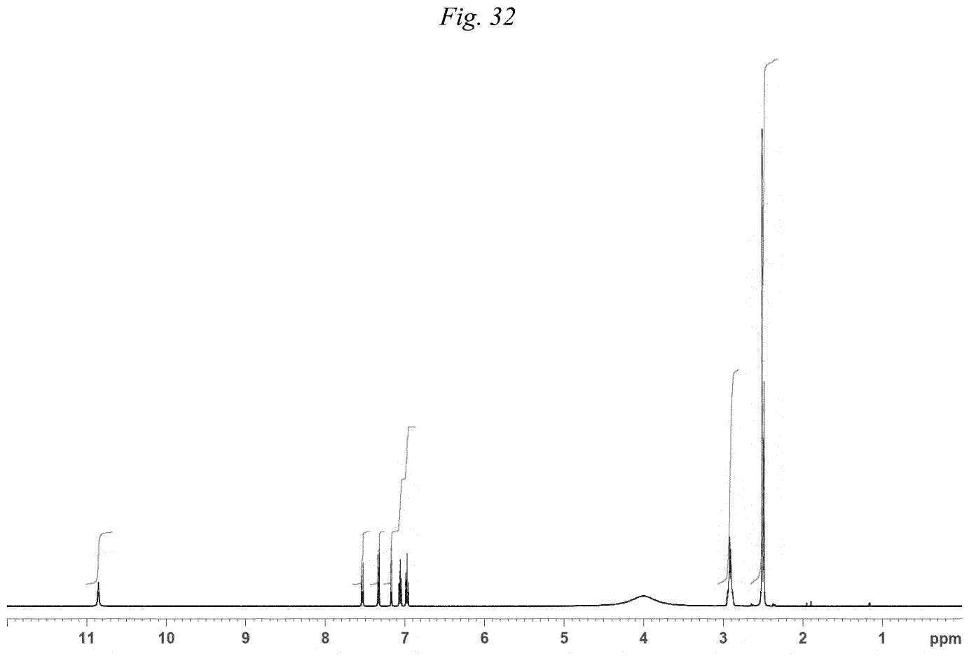

¹H nuclear magnetic resonance (NMR) spectra of Example 1 (I-1a; fumarate), Example 2 (I-1b; benzoate), Example 3 (I-1c; salicylate), Example 5 (I-1e; oxalate), Example 8 (I-1h; hemi-fumarate), Example 6 (I-1f; glycolate), Example 4 (I-1d; succinate), and Example 7 (I-1g; hemi-oxalate) are shown in FIGS. 25-32, respectively. In each case, the spectra are consistent with the proposed molecular structures with respect to chemical shifts, multiplicities and integrals. The signal integral values of counter-ion resonances are compared with those of the DMT signals to confirm the stoichiometries as stated (i.e., 2:1 DMT:counter-ion for hemi-salts, and 1:1 DMT:counter-ion for all others).

### Single Crystal X-ray Diffraction

The benzoate salt of 2-(1H-indol-3-yl)-N,N-bis(methyl-d₃)ethan-1-amine-1,1,2,2-d₄ (I-8b) was crystallized slowly from a mixture of ethanol/water through controlled evaporation, over the period of approximately 1 week. A single crystal of 2-(1H-indol-3-yl)-N,N-bis(methyl-d₃)ethan-1-amine-1,1,2,2-d₄ (I-8b) (C₁₉H₁₂D₁₀N₂O₂) was selected and mounted on a Mitegen head with Fomblin oil. This was placed on a Rigaku Oxford Diffraction Synergy-S diffractometer with a dual source equipped with a Hybrid pixel array detector. The crystal was kept at 100(2) K during data collection. Using Olex2 (Dolomanov, O. V., Bourhis, L. J., Gildea, R. J, Howard, J. A. K. & Puschmann, H. (2009), J. Appl. Cryst. 42, 339-341), the structure was solved with the SHELXT (Sheldrick, G. M. (2015). Acta Cryst. A71, 3-8) structure solution program using Intrinsic Phasing and refined with the SHELXL (Sheldrick, G. M. (2015). Acta Cryst. C71, 3-8) refinement package using Least Squares minimization.

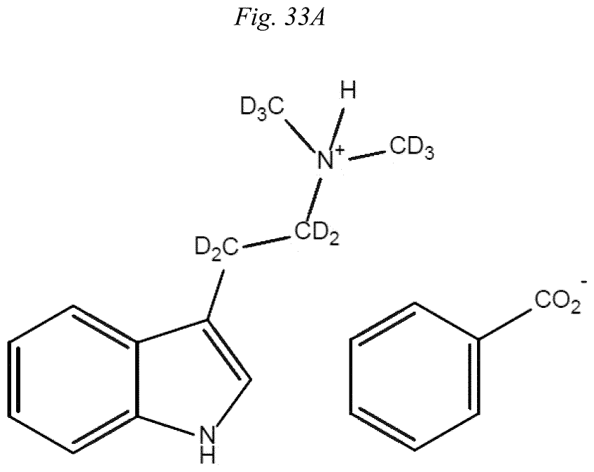

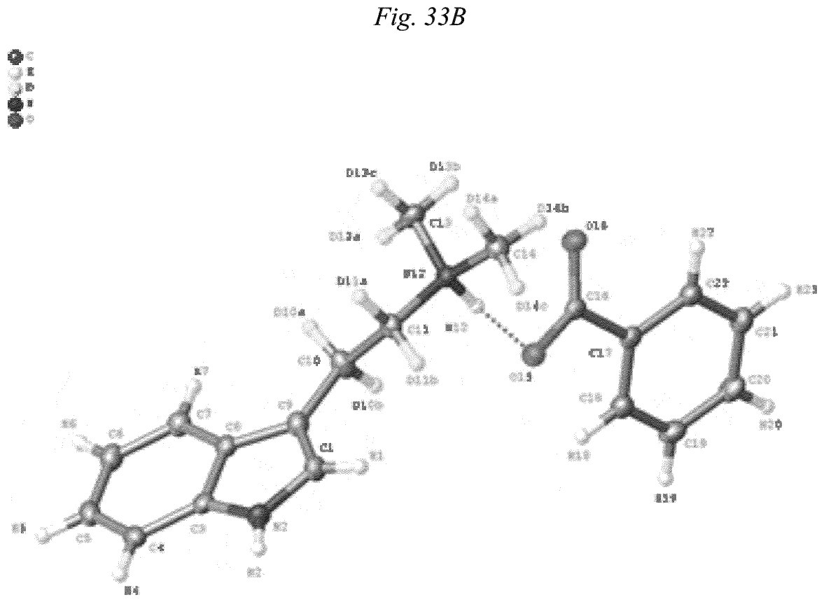

The solid-state structure of I-8b was generated; the molecular structure is shown in FIG. 33A. The asymmetric unit contains the DMT d-10 and benzoate counter-ion; there are two compounds in the unit cell connected by an inversion center as shown in FIG. 33B. The NH moieties were detected in a difference map on N2 and N12. N2 was refined at a calculated position. These NH moieties form short contacts with the benzoate counter ion listed below in Table 13.

TABLE 13
Hydrogen bonding parameters, in the asymmetric unit

| D-H | H . . . A | D . . . A | <(DHA) | Bond descriptor |
|---|---|---|---|---|
| 0.88 | 1.91 | 2.7859(13) | 175.2 | N2-H2 . . . O16 $1 |
| 0.987(17) | 1.681(17) | 2.6652(12) | 174.1(15) | N12-H12 . . . O15 |

Symmetry operator used to generate symmetry equivalent atoms in above contact was $1 1-X,2-Y,1-Z

A summary of the crystal structure data for I-8b is listed in Table 14. The proposed crystal structure has a very high degree of certainty and is consistent with expectation in accordance with the determined counter-ion stoichiometry.

TABLE 14
Crystal data and structure refinement for I-8b

| Empirical formula | C₁₉H₁₂D₁₀N₂O₂ |
|---|---|
| Formula weight | 320.45 |
| Temperature/K | 100(2) |
| Crystal system | triclinic |
| Space group | P-1 |
| a/Å | 9.1471(2) |
| b/Å | 9.4103(2) |
| c/Å | 10.6632(2) |
| α/° | 89.812(2) |
| β/° | 68.675(2) |
| γ/° | 78.044(2) |
| Volume/Å3 | 833.85(3) |
| Z | 2 |
| ρcalc g/cm³ | 1.276 |
| μ/mm⁻¹ | 0.642 |
| F(000) | 332.0 |
| Crystal size/mm³ | 0.18 × 0.12 × 0.02 colorless block |
| Radiation | CuKα (λ = 1.54184) |
| 2Θ range for data | 8.93 to 159.61 |
| collection/° | |
| Index ranges | −10 ≤ h ≤ 11, −12 ≤ |
| | k ≤12, −13 ≤ 1 ≤ 13 |
| Reflections collected | 33395 |
| Independent reflections | 3559 [Rint = 0.0762, Rsigma = 0.0293] |
| Data/restraints/parameters | 3559/0/213 |
| Goodness-of-fit on F2 | 1.065 |
| Final R indexes [I >= 2σ (I)] | R₁ = 0.0404, wR₂ = 0.1063 |
| Final R indexes [all data] | R₁ = 0.0441, wR₂ = 0.1087 |
| Largest diff. peak/hole / e Å⁻³ | 0.22/-0.23 |

### Storage Stability Assessment

The storage stability of Example 26 (I-8a; fumarate salt of 2-(1H-indol-3-yl)-N,N-bis(methyl-d₃)ethan-1-amine-1,1,2,2-d₄) was assessed under three different storage conditions, −20° C., 25° C. 60% RH, and 40° C. 75% RH, according to appearance, chemical purity by ITPLC, and quantitative NMR (qNMR). The samples were placed into the following primary container and secondary container: primary container—10 mL clear glass vial, black lid with reflective insert on ribbed lid; secondary container—amber 100 mL glass vial with black lid containing white insert.

TABLE 15

| | | | |
|---|---|---|---|
| | | Chemical | qNMR |
| Condition | Appearance | Purity (%) | (%) |

Initial Time Point

| | | | |
|---|---|---|---|
| N/A | Pale orange | 99.3 | 99.7 |
| | crystalline powder | | |

2 Week Time Point

| | | | |
|---|---|---|---|
| −20° C. | Pale orange | 99.0 | 101.2 |
| | crystalline powder | | |
| 25° C. 60% RH | Pale orange | 99.2 | 99.7 |
| | crystalline powder | | |
| 40° C. 75% RH | Pale orange | 99.0 | 100.1 |
| | crystalline powder | | |

4 Week Time Point

| | | | |
|---|---|---|---|
| −20° C. | Pale orange | 99.5 | 96.8 |
| | crystalline powder | | |
| 25° C. 60% RH | Pale orange | 99.4 | 97.1 |
| | crystalline powder | | |
| 40° C. 75% RH | Pale orange | 99.4 | 97.9 |
| | crystalline powder | | |

Table 15 shows the generated stability data. There was no significant change in any of the testing parameters at either the 2 week or 4 week time point under the tested conditions. According to the Arrhenius equation, chemical reaction rate approximately doubles with every 10° C. temperature increase. Therefore, the stability of I-8a at 40° C. for at least 1 month indicates that the material has a suitable shelf life of at least 1 year when stored at −20° C.

The preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. A pharmaceutically acceptable salt of a compound of Formula (I), or a solvate thereof, 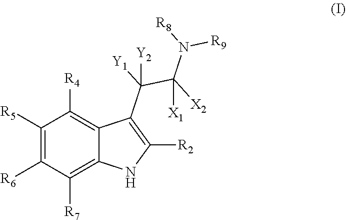
wherein:
X₁ and X₂ are deuterium,
Y₁ and Y₂ are deuterium,
R₂, R₄, R₅, R₆, and R₇ are independently hydrogen or deuterium, and
R₈ and R₉ are independently selected from the group consisting of —CH₃, —CDH₂, —CD₂H, and —CD₃.

2-6. (canceled)

7. The pharmaceutically acceptable salt of claim 1, wherein R₈ and R₉ are —CD₃.

8. (canceled)

9. The pharmaceutically acceptable salt of claim 1, wherein the compound of Formula (I) is

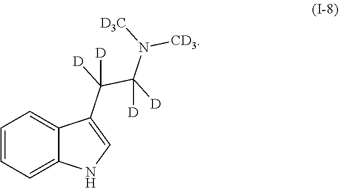 [SMILES: [2H]C([2H])([2H])N(C([2H])([2H])[2H])C([2H])([2H])C([2H])([2H])c1c[nH]c2ccccc12]

10. The pharmaceutically acceptable salt of claim 1, which is crystalline, as determined by X-ray powder diffraction (XRPD).

11. The pharmaceutically acceptable salt of claim 1, which has a water solubility from about 10 mg/mL to about 400 mg/mL.

12. The pharmaceutically acceptable salt of claim 1, which has one or more of:
(i) a melt onset from about 100° C. to about 210° C., as determined by differential scanning calorimetry (DSC);
(ii) an enthalpy of fusion from about 110 J·g⁻¹ to about 180 J·g⁻¹ as determined by differential scanning calorimetry (DSC); and
(iii) a weight increase of less than 1% w/w when exposed to a relative humidity (RH) of >95% RH, as determined by dynamic vapor sorption (DVS).

13-15. (canceled)

16. The pharmaceutically acceptable salt of claim 1, which is a fumarate, a benzoate, a salicylate, or a succinate salt of the compound of Formula (I).

17-20. (canceled)

21. The pharmaceutically acceptable salt of claim 1, which is a fumarate salt of 2-(1H-indol-3-yl)-N,N-bis(methyl-d₃)ethan-1-amine-1,1,2,2-d₄ (I-8a).

22. The pharmaceutically acceptable salt of claim 21, which is crystalline and characterized by an X-ray powder diffraction pattern containing at least three characteristic peaks at diffraction angles (2θ±0.2°) selected from 7.8°, 10.3°, 10.9°, 13.6°, 15.8°, 16.1°, 17.0°, 18.4°, 19.7°, 19.9°, 20.6°, 21.3°, 21.8°, 22.5°, 23.8°, 24.1°, 25.1°, 26.2°, 33.6° and 34.9°, as determined by XRPD using a CuKα radiation source.

23. The pharmaceutically acceptable salt of claim 1, which is a benzoate salt of 2-(1H-indol-3-yl)-N,N-bis(methyl-d₃)ethan-1-amine-1,1,2,2-d₄ (I-8b).

24. The pharmaceutically acceptable salt of claim 23, which is crystalline and characterized by an X-ray powder diffraction pattern containing at least three characteristic peaks at diffraction angles (2θ±0.2°) selected from 9.6°, 11.10, 12.7°, 13.5°, 15.8°, 16.1°, 17.2°, 17.9°, 19.8°, 20.1°, 20.8°, 21.2°, 22.8°, 23.8°, 24.6°, 26.9°, 29.3°, 32.3°, 35.1°, and 36.10, as determined by XRPD using a CuKα radiation source.

25. The pharmaceutically acceptable salt of claim 1, which is a salicylate salt of 2-(1H-indol-3-yl)-N,N-bis(methyl-d₃)ethan-1-amine-1,1,2,2-d₄ (I-8c).

26. The pharmaceutically acceptable salt of claim 25, which is crystalline and characterized by an X-ray powder diffraction pattern containing at least three characteristic peaks at diffraction angles (2θ±0.2°) selected from 9.6°, 10.5°, 14.9°, 17.10, 18.1°, 19.1°, 20.1°, 20.8°, 21.1°, 21.3°, 24.6°, 25.6°, 28.5°, 28.8°, 29.4°, 30.3°, 31.3°, 32.1°, 33.5° and 34.4°, as determined by XRPD using a CuKα radiation source.

27. The pharmaceutically acceptable salt of claim 1, which is a succinate salt of 2-(1H-indol-3-yl)-N,N-bis(methyl-d₃)ethan-1-amine-1,1,2,2-d₄ (I-8d).

28. A pharmaceutical composition, comprising the pharmaceutically acceptable salt of claim 1 and a pharmaceutically acceptable vehicle.

29. The pharmaceutical composition of claim 28, wherein any position in the compound of Formula (I) having deuterium has a minimum deuterium incorporation of at least 50 atom % at the site of deuteration.

30-31. (canceled)

32. The pharmaceutical composition of claim 28, which is adapted for intravenous, subcutaneous, or intramuscular administration.

33-35. (canceled)

36. A liquid dosage form, prepared by reconstituting a solid dosage form comprising the pharmaceutically acceptable salt of claim 1 in a pharmaceutically acceptable liquid medium.

37-53. (canceled)

54. A method of treating a patient with a central nervous system (CNS) disorder and/or psychological disorder, comprising:
administering to the patient a therapeutically effective amount of the pharmaceutically acceptable salt of claim 1.

55. The method of claim 54, wherein the CNS disorder and/or psychological disorder is at least one selected from the group consisting of post-traumatic stress disorder (PTSD), major depressive disorder (MDD), treatment-resistant depression (TRD), suicidal ideation, suicidal behavior, melancholic depression, atypical depression, dysthymia, non-suicidal self-injury disorder (NSSID), bipolar and related disorders, obsessive-compulsive disorder (OCD), generalized anxiety disorder (GAD), acute psychedelic crisis, social anxiety disorder, alcohol use disorder, opioid use disorder, amphetamine use disorder, nicotine use disorder, cocaine use disorder, Alzheimer's disease, cluster headache and migraine, attention deficit hyperactivity disorder (ADHD), pain, aphantasia, childhood-onset fluency disorder, major neurocognitive disorder, mild neurocognitive disorder, chronic fatigue syndrome, Lyme disease, gambling disorder, anorexia nervosa, bulimia nervosa, binge-eating disorder, pedophilic disorder, exhibitionistic disorder, voyeuristic disorder, fetishistic disorder, sexual masochism or sadism disorder, transvestic disorder, sexual dysfunction, and obesity.

56. The method of claim 54, wherein the CNS disorder and/or psychological disorder is opioid use disorder.

57. The method of claim 54, wherein the CNS disorder and/or psychological disorder is generalized anxiety disorder (GAD).

58. The method of claim 54, wherein the CNS disorder and/or psychological disorder is social anxiety disorder.

59. The method of claim 54, wherein the CNS disorder and/or psychological disorder is a depressive disorder.

60. A pharmaceutical composition, comprising:
an active salt mixture comprising (i) a pharmaceutically acceptable salt of 2-(1H-indol-3-yl)-N,N-bis(methyl-d₃)ethan-1-amine-1,1,2,2-d₄ (I-8), and (ii) a pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11); and
a pharmaceutically acceptable vehicle.

61. The pharmaceutical composition of claim 60, wherein the active salt mixture comprises (i) from 60% to 98% by weight of the pharmaceutically acceptable salt of 2-(1H-indol-3-yl)-N,N-bis(methyl-d₃)ethan-1-amine-1,1,2,2-d₄ (I-8), based on a total weight of the active salt mixture, and (ii) from 2% to 40% by weight, in sum, of the pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), based on a total weight of the active salt mixture.

62. The pharmaceutical composition of claim 60, wherein the active salt mixture comprises (i) from 90% to 98% by weight of a pharmaceutically acceptable salt of 2-(1H-indol-3-yl)-N,N-bis(methyl-d₃)ethan-1-amine-1,1,2,2-d₄ (I-8), based on a total weight of the active salt mixture, and (ii) from 2% to 10% by weight, in sum, of the pharmaceutically acceptable salt of one or more of 2-(1H-indol-3-yl)-N,N-bis(methyl-d₃)ethan-1-amine-1,2,2-d₃ (I-10) and/or 2-(1H-indol-3-yl)-N,N-bis(methyl-d₃)ethan-1-amine-1,1,2-d₃ (I-11), based on a total weight of the active salt mixture.

63. The pharmaceutical composition of claim 60, wherein the active salt mixture comprises (i) a fumarate salt of 2-(1H-indol-3-yl)-N,N-bis(methyl-d₃)ethan-1-amine-1,1,2,2-d₄ (I-8a), and (ii) a fumarate salt of one or more of 2-(1H-indol-3-yl)-N,N-bis(methyl-d₃)ethan-1-amine-1,2,2-d₃ (I-10a) and/or 2-(1H-indol-3-yl)-N,N-bis(methyl-d₃)ethan-1-amine-1,1,2-d₃ (I-11a).

64-66. (canceled)
